SEQUENCE LISTING (137 sequences)

SEQ ID NO: 1 - protein, 528 aa, Artificial Sequence
  Synthetic amino acid sequence of the enzymes able to catalyze the decarboxylation of oxaloacetate into malonic semi-aldehyde
  VARIANT 109: Xaa in position 109 is selected from the group consisting of leucine, lysine, arginine and valine
  VARIANT 110: Xaa in position 110 is selected from the group consisting of leucine and lysine
  VARIANT 377: Xaa in position 377 is selected from the group consisting of threonine and serine
  VARIANT 397: Xaa in position 397 is selected from the group consisting of phenylalanine, asparagine, alanine, isoleucine and valine
  VARIANT 398: Xaa in position 398 is selected from the group consisting of cysteine and arginine
  VARIANT 403: Xaa in position 403 is selected from the group consisting of leucine, asparagine and alanine
  VARIANT 460: Xaa in position 460 is selected from the group consisting of alanine and leucine
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEAXXTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHQYDPGQYLKPGTRLISVTCDPLEAARAPMGDAIVADIGAMASALANLVEESSRQLPTAAPEPAKVDQDAGRLHPETVFDTLNDMAPENAIYLNESXSTTAQMWQRLNMRNPGSYYXXAAGGXGFALPAAIGVQLAEPERQVIAVIGDGSANYSISALWTAAQYNIPTIFVIMNNGTYGXLRWFAGVLEAENVPGLDVPGIDFRALAKGYGVQALKADNLEQLKGSLQEALSAKGPVLIEVSTVSPVK

SEQ ID NO: 2 - protein, 528 aa, Artificial Sequence
  Synthetic enzyme 1
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGALRWFAGVLEAENVPGLDVPGIDFRALAKGYGVQALKADNLEQLKGSLQEALSAKGPVLIEVSTVSPVK

SEQ ID NO: 3 - protein, 528 aa, Artificial Sequence
  Synthetic Enzyme 2
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYNCAAGGLGFALPAAIGVQLAEPERQVIAVIGDGSANYSISALWTAAQYNIPTIFVIMNNGTYGALRWFAGVLEAENVPGLDVPGIDFRALAKGYGVQALKADNLEQLKGSLQEALSAKGPVLIEVSTVSPVK

SEQ ID NO: 4 - protein, 528 aa, Artificial Sequence
  Synthetic Enzyme 3
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEAKLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYNCAAGGLGFALPAAIGVQLAEPERQVIAVIGDGSANYSISALWTAAQYNIPTIFVIMNNGTYGALRWFAGVLEAENVPGLDVPGIDFRALAKGYGVQALKADNLEQLKGSLQEALSAKGPVLIEVSTVSPVK

SEQ ID NO: 5 - protein, 528 aa, Artificial Sequence
  Synthetic Enzyme 4
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYNCAAGGLGFALPAAIGVQLAEPERQVIAVIGDGSANYSISALWTAAQYNIPTIFVIMNNGTYGLLRWFAGVLEAENVPGLDVPGIDFRALAKGYGVQALKADNLEQLKGSLQEALSAKGPVLIEVSTVSPVK

SEQ ID NO: 6 - protein, 528 aa, Artificial Sequence
  Synthetic Enzyme 5
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEAVKTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYNCAAGGNGFALPAAIGVQLAEPERQVIAVIGDGSANYSISALWTAAQYNIPTIFVIMNNGTYGALRWFAGVLEAENVPGLDVPGIDFRALAKGYGVQALKADNLEQLKGSLQEALSAKGPVLIEVSTVSPVK

SEQ ID NO: 7 - protein, 248 aa, Escherichia coli K-12
  YDFG Escherichia Coli BAA15241.1 L-allo-threonine dehydrogenase NAD(P)-binding
MIVLVTGATAGFGECITRRFIQQGHKVIATGRRQERLQELKDELGDNLYIAQLDVRNRAAIEEMLASLPAEWCNIDILVNNAGLALGMEPAHKASVEDWETMIDTNNKGLVYMTRAVLPGMVERNHGHIINIGSTAGSWPYAGGNVYGATKAFVRQFSLNLRTDLHGTAVRVTDIEPGLVGGTEFSNVRFKGDDGKAEKTYQNTVALTPEDVSEAVWWVSTLPAHVNINTLEMMPVTQSYAGLNVHRQ

SEQ ID NO: 8 - protein, 1824 aa, Chloroflexus aggregans
  Propionyl-CoA synthase Chloroflexus aggregans
MAIDTAPPAPSRAPRSNPIRDRVDWETQRAAALANPGAFHGAIARTVIHWYDPKHNCWIRFDESSQRWEGLDAATGAPVTVDYPADYQPWQQAFDDSEAPFYRWFYGGLTNACFNEVDRHVMMGYGDEVAYYFEGDRWDNSLNNGRGGPVVQETITRRRLLVEVVKAAQVLRDLGLKKGDRIALNMPNIMPQIYYTEAAKRLGIIYTPVFGGFSDKTLSDRIHNAGARVVITSDGAYRNAQVVPYKEAYTDQALDKYVPVETVRRIVAETLTTLPLRDDQRQKIIDEVESVLSGEITIERSDVMRGVGAALAQIRDLDAATQANVRTVLAQALVASPPRVEAVIVVRHTGQDILWNADRDRWSHELLEAALAKILANARAIGIDVHSEADLLKLPDDQLIRALYASVPCEPVDAEYPMFIIYTSGSTGKPKGVIHVHGGYVSGVVHTLRVSFDAEPGDTIYVIADPGWITGQSYMLTATMAGRLTGIIAEGSPLFPSSGRYASIIERYGVQIFKAGVTFLKTVMSNPQNIEDVRLYNMSSLRVATFCAEPVSPAVQQFGMQIMTPQYINSYWATEHGGIVWTHFYGNNDFPLRPDAHTYPLPWVAGDVWVAETDESGNVRYRVADYEEKGEIVITAPYPYLTRTIWGDVPGFEAYLRGEIPLKAWKGDAERFIKTYWRRGPHGEWGYIQGDFAMKYPDGSFTLHGRSDDVINVSGHRMGTEEIEGAILRDRQITPDSPVGNCIVVGAPHREKGLTPVAFIQPAPGRRLTAADRRRLDELVRTEKGAVSVPEDYIEVSAFPETRSGKYMRRFLRNMMLDEPLGDTTTLRNPEVLEEIAAKIAEWKRKQRLAEEQQIIERYRYFRIEYHPPAASAGKLAVVTVTNPPVNALNERALDELNTIVDHLSRRNDVAAIVFTGQGAKSFVAGADIRQLLEEIHTVEEAMALPNNAHLAFRKIERMNKPCIAAINGVALGGGLEFAMACHYRVADVYAEFGQPEINLRLLPGYGGTQRLPRLLHRRSNGTGLLRALEMILGGRSVPADEALELGLIDAIATGDADALMLACSLAREAIASDGTLREDAAVTKAFRQRHAQLEEWRKPDPHFTDDQLRSIIAHPRIERIIKQAHTVGRDVAVHRALDAIRYGFIHGFEAGLEHEAKLFAEAVVDPNGGKRGIREFLDRHSAPLPTRRPLISREQEQLLLEQKELLPIGSPFFPGVDRIPKWQYAQAFIRDPETGAAMHGDPIVAEKQIIIPVERPRANQALIYVLASEVNFNDIWAITGIPVSRFDEHDRDWHVTGSGGIGLVVALGEEARREGRLKIGDLVAIYSGQTDLLSPLMGLDPMAADFVIQGNDTPDGSHQQFMVAQAPQCMPVLPDMTLEAAGSYILNLGTIYRALFTTLRVQPGRTIFIEGAATGTGLDAVRTAARNGLNVIGMVSSPSRAATVLSAGGKGAINRKDPAIANCFTRVPEDPSEWAAWEAAGHPLLEMFRAQNGGRLADYVVSHAGETAFPRSFQLLGEPHDGHIPTLTFYGASSGYHFTFIGKPGAASPTEMLRRAGLRAGEGVLIYYGVGSPELIDGAGLEAIEAARLMGARIVVVTASDAQREFVLSLGFGAALRGVISITELKRRFGDEFDWPKTMPPLPDSRKDPQGLKEAVRRFNDLTFKPIGSAVGGFLRTPDNPRGYPDLIIERAGHDALAVSAMLIKPFTGRIVYFEDIGGRRYSFFAPQIWVRQRRIYMPTAQIFGTHLSNAYEIVRMNEEISAGLITITEPAVVPWDQLPEAHQAMWENRHTAATYVVNHALPRLGIKTKDELYEAWTAADRE

SEQ ID NO: 9 - protein, 1912 aa, Roseiflexus castenholzii
  Propionyl-CoA synthase Roseiflexus castenholzii
MTTIESALRPPRINPVRTRADWEAQRKAALTDPGAFHGAIARSAIHWYDRHLDAWITWDEEEGCWKGLRYSDGAPIDVPYGPDHEPWERAFNGDDPPFYRWFEGGLTNACFNEVDRHVLTGYGDEVAFYFEGDRWDSSLNNGRGGPVVSFAVTRKQLMLEVVKAAQVLRDLGLNMGDRIALNMPNIMEQLYYTEAAKRLGIIYTPVFGGFSDKTLSDRIHNAGARLVITSDGAYRNAQVVPYKEQYTDQALDKFVPVETALDIIEAALSGSEGSPVPGAPLLAPDQIKHILAQVRDALKEDITIERSDAMRAVGRAIEGLTGVDALAQSRARTAVAQALVNTPPRVDAVIVVRHTGQDILWRPERDRWSHELTARALETILANARAVGVEVYSEDELLNLPTDQFVKALYATSRAEPLDAEYPMFIIYTSGSTGKPKGVVHVHGGYVAGVAYTMRVSFDAEPGDTIYVVADPGWITGQSYMICATLTTRCTGIITEGSPVFPSAGRFASIIERYKVRIFKAGVTFLKTVMSDPQNTADARQYDMSSLRVCTFCAEPVSPAVQQFGMELMSPQYINSYWATEHGGIVWTHFYGNEDFPLRPDAHTYPLPWIAGEVWVLEGGDRDATGAEAPRYRIADYEEKGEIVITAPYPYLTRTIWGDVKGFEAWVAAMQHGNGATAPRWRGDAERFIKTYWRRGPNDEWGYIQGDFAMKYPDGSFTLHGRSDDVINVSGHRMGTEEIEGAILRDKIITPDSPVGNCIVVGAPHREKGLTPVAFILTAPGRKLTGEDRRRLNELVRNEKGAVSVPEDYIEVSAFPETRSGKYMRRFLRNLMLGEPLGDTTTLRNPESLKEIAEKIEAWKRKQRMAEEQRIFERYRYFRIEYHAVRERWTPSGNGSAAAEQKALTQRIAIVTVTNPPVNALNERALDELNTIVDHLARREDVAAVVFTGSGTKSFVAGADIKQMLEEMHTVEDAMALPNNAHLAFRKIETMNKPCIAAINGVALGGGMEFALACHYRIADLHAEFGQPEINLRLLPGYGGTQRLPRLLYSRRGEAGLIKALMIIMGGRTLNAERAYEIGLIDKVAHGHEEALTLATQMAREMILAERNGKPTELRVIPNTSDDPTTFFSPFHDPATAGEGLWPPLPEAYALRRQLTAQWETPDPAMVDLLEKALRDPLITRIINQAQWAGRARAIERIIDALRTGFTKGMLAGLEREARLFAEAVVAPDEGKIGIQDFLDKRSAPLPTRRRIHLTPDEEKRMIDAGMLLPIGAPFFPGVTPIPVAQYAMAVVRDEVTGAPAHGDPIEAEKQIIIPVEKPGPNDVLLYILASEVNFNDIWAITGVPVSQFDEHDRDWHVTGSGGIGLIVSVGEETKREGRLKVGDLVAIYSGQNDLLSPMVGLDPMAADFVIQGYNTPDASHQQFMVAQAPQCFPVLPDLTLEAAGSYMLNLGTVYRALFTTLKIQPGRRIFVEGAATGTGLDAARSAARNGLYVTGMVSSQERAAVVRAAGAVGVINRRDPRYAGIFTRVPEDPAKWAEWEAAGRPLLEDYRAQNGGHLADYAVSHAGETAFPRSFQLLGEPHDGHIPTLTFYGASSGYHFTFLGKPGAADPVEMLRRAGLRAGEAVMIYYGVDDRSYLGDEGYESGAVPETLERRTTLVDQVGLEAIESARAMGARIVVVTYTDAQREFVLSLGFGASLKGVVSLEELHRRYGDEFDWPETMPPLPDAKSDIHGFKAAVRRFNDLTFKPLGTAVGQFLRSNDNPRGYPDLIIERSGHDTLAVSVMLIKPFTGRVVYFENMDGQRYSFFAPQVWMRQRRIYMPTANIWGTHLSNAYEIVRLNDEISAGLLSITEPTLVEWNDLPQAHQAMWENRHQGATYVVNHALPRPGLKDKDELYEAWSEMLQNREYGV

SEQ ID NO: 10 - protein, 1823 aa, Chloroflexus aurantiacus
  Propionyl-CoA synthase Chloroflexus aurantiacus
MAIDTAPLAPPRAPRSNPIRDRVDWEAQRAAALADPGAFHGAIARTVIHWYDPQHHCWIRFNESSQRWEGLDAATGAPVTVDYPADYQPWQQAFDDSEAPFYRWFSGGLTNACFNEVDRHVTMGYGDEVAYYFEGDRWDNSLNNGRGGPVVQETITRRRLLVEVVKAAQVLRDLGLKKGDRIALNMPNIMPQIYYTEAAKRLGILYTPVFGGFSDKTLSDRIHNAGARVVITSDGAYRNAQVVPYKEAYTDQALDKYIPVETAQAIVAQTLATLPLTESQRQTIITEVEAALAGEITVERSDVMRGVGSALAKLRDLDASVQAKVRTVLAQALVESPPRVEAVVVVRHTGQEILWNEGRDRWSHDLLDAALAKILANARAAGFDVHSENDLLNLPDDQLIRALYASIPCEPVDAEYPMFIIYTSGSTGKPKGVIHVHGGYVAGVVHTLRVSFDAEPGDTIYVIADPGWITGQSYMLTATMAGRLTGVIAEGSPLFPSAGRYASIIERYGVQIFKAGVTFLKTVMSNPQNVEDVRLYDMHSLRVATFCAEPVSPAVQQFGMQIMTPQYINSYWATEHGGIVWTHFYGNQDFPLRPDAHTYPLPWVMGDVWVAETDESGTTRYRVADFDEKGEIVITAPYPYLTRTLWGDVPGFEAYLRGEIPLRAWKGDAERFVKTYWRRGPNGEWGYIQGDFAIKYPDGSFTLHGRSDDVINVSGHRMGTEEIEGAILRDRQITPDSPVGNCIVVGAPHREKGLTPVAFIQPAPGRHLTGADRRRLDELVRTEKGAVSVPEDYIEVSAFPETRSGKYMRRFLRNMMLDEPLGDTTTLRNPEVLEEIAAKIAEWKRRQRMAEEQQIIERYRYFRIEYHPPTASAGKLAVVTVTNPPVNALNERALDELNTIVDHLARRQDVAAIVFTGQGARSFVAGADIRQLLEEIHTVEEAMALPNNAHLAFRKIERMNKPCIAAINGVALGGGLEFAMACHYRVADVYAEFGQPEINLRLLPGYGGTQRLPRLLYKRNNGTGLLRALEMILGGRSVPADEALELGLIDAIATGDQDSLSLACALARAAIGADGQLIESAAVTQAFRHRHEQLDEWRKPDPRFADDELRSIIAHPRIERIIRQAHTVGRDAAVHRALDAIRYGIIHGFEAGLEHEAKLFAEAVVDPNGGKRGIREFLDRQSAPLPTRRPLITPEQEQLLRDQKELLPVGSPFFPGVDRIPKWQYAQAVIRDPDTGAAAHGDPIVAEKQIIVPVERPRANQALIYVLASEVNFNDIWAITGIPVSRFDEHDRDWHVTGSGGIGLIVALGEEARREGRLKVGDLVAIYSGQSDLLSPLMGLDPMAADFVIQGNDTPDGSHQQFMLAQAPQCLPIPTDMSIEAAGSYILNLGTIYRALFTTLQIKAGRTIFIEGAATGTGLDAARSAARNGLRVIGMVSSSSRASTLLAAGAHGAINRKDPEVADCFTRVPEDPSAWAAWEAAGQPLLAMFRAQNDGRLADYVVSHAGETAFPRSFQLLGEPRDGHIPTLTFYGATSGYHFTFLGKPGSASPTEMLRRANLRAGEAVLIYYGVGSDDLVDTGGLEAIEAARQMGARIVVVTVSDAQREFVLSLGFGAALRGVVSLAELKRRFGDEFEWPRTMPPLPNARQDPQGLKEAVRRFNDLVFKPLGSAVGVFLRSADNPRGYPDLIIERAAHDALAVSAMLIKPFTGRIVYFEDIGGRRYSFFAPQIWVRQRRIYMPTAQIFGTHLSNAYEILRLNDEISAGLLTITEPAVVPWDELPEAHQAMWENRHTAATYVVNHALPRLGLKNRDELYEAWTAGER

SEQ ID NO: 11 - protein, 865 aa, Clostridium beijerinckii
  ADHE2 Clostridium beijerinckii
MARVTNPEELTKRIEQIREAQREFAKFSQEEVDEIFRQAAMAANNARITLAKMAVEESGMGIVEDKVIKNHFAAEYIYNQYKDTKTCGVIERDEMFGITHIAEPIGVIAAIVPTTNPTSTAIFKTLIALKTRNGIIISPHPRAKNSTIAAAKIVLEAAERAGAPKGIIGWIDEPSIELSRNVMAESDIILATGGPGMVRAAYSSGKPAIGVGAGNTPAIIDDTAHIKMAVNSILLSKTFDNGVVCASEQSIIAMESVYDEVLKELDERGAYILKGDEVDKVRSIILDSKGSLNSEIVGQSAYKIAKMAGVEISEAVKVLIGEVESPELEEPFSHEKLSPILGMYKAKTFDDALRLASRMIELGGFGHTSILYTNQVESVDRIEKFGVAMKTARTLINMPASQGAIGDIYNFKLAPSLTLGCGSWGGNSISENVGPKHLINVKRIAERRENMLWFRVPDKIYFKFGCLPVALEELNAMKKKRAFIVTDRVLFDLGYTHKITNILSENHIEYKIFSDVEPDPTLKAAKLGADAMRDFNPDVIIAIGGGSPMDAAKIMWVMYEHPDVRFEDLAMRFMDIRKRVYEFPPMGEKAILVAIPTSAGTGSEVTPFAVITDQQTGVKYPLADYALTPNMAIIDAELMMSMPKGLTAASGIDALVHAIEAYVSVLASEYTNGLALEAIRLTFKYLPDAYNGGTTNIKAREKMAHASSVAGMAFANAFLGICHSMAHKLGAFHHVPHGIANALLIDEVIRFNATDAPRKQAAFPQYKYPNAGWRYARIADYLNLGGNTEEEKVELLIKAIDDLKGKVGIPKSIKEFGVSEEKFYASMDEMVEQAFDDQCTGANPRYPLMSEIKEMYIKSYNVSNK

SEQ ID NO: 12 - protein, 869 aa, Clostridium arbusti
  ADHE from Clostridium arbusti
MAKVSNIDELNVRLEEIREAQRKFGTYTQEQVDEIFRQAAMAALDARIPLAKMAAEETGMGLVEDKVIKNHFAAEYIYNQYKDEKTCGVVETDKSYGITKIAEPIGIVAAVIPTTNPTSTAIFKTLISLKTRNAIMLSPHPRAKKSTIAAAKIILDAAVKAGAPEGIIGWIDEPSIELTQILMQEADITLATGGPSMVKSAYSSGKPAIGVGPGNTPVIIDESAHIKMAVSSVILSKTFDNGVICASEQSVIVLDSIYDEVRKEFAERGAYIIKESEIDKVRKTIFINGSINSKIVGQSAYKIAEMSGIKVPETARILIGEVTSVGKEEPFAHEKLSTVLAMYRAENFDDALDKAVTLVNLGGLGHTSAIYADIIKAKDKIDKFSNAMKTVRTFINIPAAQGASGDLYNFKIAPSFTLGCGSWGGNSVSENVGPKHLLNIKRVAERRENMLWFRVPEKVYFKFGCLQFALRELKDLNKKRAFIVTDKVLYDLGYADAITKVLEEIGVDFKVFTEVEPDPTLSTARKGTEEMMDFKPDTIISLGGGSAMDAAKIMWVMYEHPEVKFEDLAMRFMDIRKRIYNFPKLGEKAMMIAVATSAGTGSEVTPFAVITDEKTGVKYPLADYELTPNMAIVDAELMMNMPKGLTAASGIDALIHGIEAYTSVLASEYTNGLALEAIRLIFKYLPTAYAEGTTNEKAREKMAHASTMAGMAFANAFLGVCHSMAHKLGAEHHIAHGTANALLIEEVIRFNSADNPVKQAAYPQYKYPNAKWRYGKIADYLNLGGNTDDEKVELLIKAIHELKEKINIPMSIKDAGVSEKNFYATLDKMCELAFDDQCTGANPRYPLISEIKQMLITAFDKTEINTEIK

SEQ ID NO: 13 - DNA, 554 bp, Saccharomyces cerevisiae
  pTDH3
  pTDH3
ccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 14 - DNA, 550 bp, Saccharomyces cerevisiae
  pENO2
  pENO2
cgctcagcatctgcttcttcccaaagatgaacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 15 - DNA, 419 bp, Saccharomyces cerevisiae
  pTEF Kl
  pTEF Kl
ctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaa

SEQ ID NO: 16 - DNA, 598 bp, Saccharomyces cerevisiae
  pTEF3
  pTEF3
ggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 17 - DNA, 383 bp, Saccharomyces cerevisiae
  pTEF1
  pTEF1
gtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctacgatacagttctcacatcacatccgaacataaacaacc

SEQ ID NO: 18 - DNA, 700 bp, Saccharomyces cerevisiae
  pADH1
  pADH1
gggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatataca

SEQ ID NO: 19 - DNA, 549 bp, Saccharomyces cerevisiae
  pGMP 1
  pGMP 1
gccaaacttttcggttaacacatgcagtgatgcacgcgcgatggtgctaagttacatatatatatatatatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaata

SEQ ID NO: 20 - DNA, 650 bp, Saccharomyces cerevisiae
  pFBA1
  pFBA1
acgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaa

SEQ ID NO: 21 - DNA, 700 bp, Saccharomyces cerevisiae
  pPDC1
  pPDC1
ttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaa

SEQ ID NO: 22 - DNA, 998 bp, Saccharomyces cerevisiae
  pCCW12
  pCCW12
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaata

SEQ ID NO: 23 - DNA, 700 bp, Saccharomyces cerevisiae
  pGK1
  pGK1
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 24 - DNA, 400 bp, Saccharomyces cerevisiae
  pSAM4
  pSAM4
agattttggtgttagatggtactcttgcatatgtaacctttaataaattttgcaaatcgaattcctttgtaacgtgcaaagcattttatagcctggcgctcgcattgttaagcaacaggcggtgcggcaacgttgaaatgtttcacgcagggttttttacgtactgcacggcattctggagtgaaaaaaaatgaaaagtacagctcgaagttttttgtccatcggttgtactttgcagagtattagtcatttttgatatcagagtactactatcgaagcatttttacgcttgaataacttgaatattattgaaagcttagttcaaccaagctgaaaagaaccattattcaacataattggaaatcatttcgttactaaatcgtccgaaaattgcagaaaa

SEQ ID NO: 25 - DNA, 505 bp, Saccharomyces cerevisiae
  pCUP1-1
  pCUP1-1
cggcaaacttcaacgatttctatgatgcattttataattagtaagccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttccagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 26 - DNA, 500 bp, Candida glabrata
  pCUP1 Cgla
  pCUP1 Cgla
cacaccacacaaccgtcagcaccccggctgtacgtctgtgaaggctgcggtatagacacggactgcgatacagaactcatgacttatatctgtagactcctctgcttcaatgcgaactccaggatcaccgaatagcatgcgatgagctgttgattcttatatataattatctattgcattttttttttaatgctgcatgggggggcctagtaaatcacccgtacaagtcacgcgtgagagaaagagaagggccctttcgtcgtggaagcgtggatcgtgagcgacctgtttctaaatatagcttttgggtaggatattatattaagtgaaattttattagagggtaaatgtatgtgaaagttatgtataatatgttgctaaattagcgatcgtgaatgcatagaatctaatcgttatagaaaaccgcaacttgtgctgttttgttgtgttttcttgtcgtttttttatattatttatctagtattttgctttagttgtta

SEQ ID NO: 27 - DNA, 425 bp, Saccharomyces bayanus
  pCUP1 Sba
  pCUP1 Sba
agaaggaggggtcctattaccaatacttggacgctatacgtgcatatgtacatgtacgtatctgtatttaaacacttttgtattattttctttatatatgtgtataggtttacatggttgacttttatcattgtttgtgcacatttgcaatggccatttttttgtttttgagaaaggtattattgctgtcactattcgagatgcttttgctgacattcctcctagaagccaaaaggccgatgcgttttttccgctgagaggataccagcaaaaaaagctaccagtacaagatgggacggcaaaagcgtataaaagaagaagcaaaatgaccagatatgctttcaatttcatcaatgtttctttctccctgttatgatccagaagaataatcaaaagcaaaacatctattcaatcaatctcataaa

SEQ ID NO: 28 - DNA, 741 bp, Artificial Sequence
  Synthetic pACU1
  pACU1
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattcgaaaaagacatttttgctgtcagtcactgtcaagagattcttttgctggcatttcttccagaagcaaaaagagcgatgcgtcttttccgctgaaccgttccagcaaaaaagactaccaacgaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 29 - DNA, 757 bp, Artificial Sequence
  Synthetic pACU2
  pACU2
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccgaattcgaaaaagacatttttgctgtcagtcactgtcaagagattcttttgctggcatttcttccagaagcaaaaagagcgatgcgtcttttccgctgaaccgttccagcaaaaaagactaccaacgaattccacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 30 - DNA, 498 bp, Artificial Sequence
  Synthetic pACU3p
  pACU3p
ttatattgaattttcaaaaattcttactttttttttggatggacgcaaagaagtttaataatcatattacatggcattaccaccatatacatatccatatctaatcttacttatatgttgtggaaatgtaaagagcccgaattcgaaaaagacatttttgctgtcagtcactgtcaagagattcttttgctggcatttcttccagaagcaaaaagagcgatgcgtcttttccgctgaaccgttccagcaaaaaagactaccaacgaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 31 - DNA, 498 bp, Artificial Sequence
  Synthetic pACU4p
  pACU4p
ttatattgaattttcaaaaattcttactttttttttggatggacgcaaagaagtttaataatcatattacatggcattaccaccatatacatatccatatctaatcttacttatatgttgtggaaatgtaaagagcccgaattcgttggtagtcttttttgctggaacggttcagcggaaaagacgcatcgctctttttgcttctggaagaaatgccagcaaaagaatctcttgacagtgactgacagcaaaaatgtctttttcgaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 32 - DNA, 530 bp, Artificial Sequence
  Synthetic pACU5
  pACU5
ggaggacgaaacaaaaaagtgaaaaaaaatgaaaatttttttggaaaaccaagaaatgaattatatttccgtgtgagacgacatcgtcgaatatgattcagggtaacagtattgatgtaatcaatttcctacctgaatctaaaattcccggaattcgaaaaagacatttttgctgtcagtcactgtcaagagattcttttgctggcatttcttccagaagcaaaaagagcgatgcgtcttttccgctgaaccgttccagcaaaaaagactaccaacgaattccgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaata

SEQ ID NO: 33 - DNA, 867 bp, Artificial Sequence
  Synthetic pACU6
  pACU6
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattcgaaaaagacatttttgctgtcagtcactgtcaagagattcttttgctggcatttcttccagaagcaaaaagagcgatgcgtcttttccgctgaaccgttccagcaaaaaagactaccaacgaattcgaaaaagacatttttgctgtcagtcactgtcaagagattcttttgctggcatttcttccagaagcaaaaagagcgatgcgtcttttccgctgaaccgttccagcaaaaaagactaccaacgaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 34 - DNA, 867 bp, Artificial Sequence
  Synthetic pACU7
  pACU7
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattcgttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttcgaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 35 - DNA, 1119 bp, Artificial Sequence
  Synthetic pACU8
  pACU8
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattcgaaaaagacatttttgctgtcagtcactgtcaagagattcttttgctggcatttcttccagaagcaaaaagagcgatgcgtcttttccgctgaaccgttccagcaaaaaagactaccaacgaattcgaaaaagacatttttgctgtcagtcactgtcaagagattcttttgctggcatttcttccagaagcaaaaagagcgatgcgtcttttccgctgaaccgttccagcaaaaaagactaccaacgaattcgaaaaagacatttttgctgtcagtcactgtcaagagattcttttgctggcatttcttccagaagcaaaaagagcgatgcgtcttttccgctgaaccgttccagcaaaaaagactaccaacgaattcgaaaaagacatttttgctgtcagtcactgtcaagagattcttttgctggcatttcttccagaagcaaaaagagcgatgcgtcttttccgctgaaccgttccagcaaaaaagactaccaacgaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 36 - DNA, 624 bp, Artificial Sequence
  Synthetic pACU9
  pACU9
tatagttttttctccttgacgttaaagtatagaggtatattaacaattttttgttgatacttttatgacatttgaataagaagtaatacaaactgaaaatgttgaaagtattagttaaagtggttatgcagcttttccatttatatatctgttaatagatcaaaaatcatcgcttcgctgattaattaccccagaaataaggctaaaaaactaatcgcattatcatccgaattcgaaaaagacatttttgctgtcagtcactgtcaagagattcttttgctggcatttcttccagaagcaaaaagagcgatgcgtcttttccgctgaaccgttccagcaaaaaagactaccaacgaattcgaaaaagacatttttgctgtcagtcactgtcaagagattcttttgctggcatttcttccagaagcaaaaagagcgatgcgtcttttccgctgaaccgttccagcaaaaaagactaccaacgaattcgggctctttacatttccacaacatataagtaagattagatatggatatgtatatggtggtaatgccatgtaatatgattattaaacttctttgcgtccatccaaaaaaaaagtaagaatttttgaaaattcaatataa

SEQ ID NO: 37 - DNA, 876 bp, Artificial Sequence
  Synthetic pACU10p
  pACU10p
ttatattgaattttcaaaaattcttactttttttttggatggacgcaaagaagtttaataatcatattacatggcattaccaccatatacatatccatatctaatcttacttatatgttgtggaaatgtaaagagcccgaattggttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttccaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 38 - DNA, 633 bp, Artificial Sequence
  Synthetic pACU11
  pACU11
gctcagcatctgcttcttcccaaagatgaacgcggcgttatgtcactaacgacgtgcaccaacttgcgggaattcgaaaaagacatttttgctgtcagtcactgtcaagagattcttttgctggcatttcttccagaagcaaaaagagcgatgcgtcttttccgctgaaccgttccagcaaaaaagactaccaacgaattccaccgcacgccttttttctgaagcccactttcgtggactttgccatatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 39 - DNA, 1119 bp, Artificial Sequence
  Synthetic pACU12
  pACU12
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattggttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttccaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 40 - DNA, 1497 bp, Artificial Sequence
  Synthetic pACU13
  pACU13
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattggttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttcgaattcgttggtagtcttttttgctggaacggttcagcggaaaagacgcatcgctctttttgcttctggaagaaatgccagcaaaagaatctcttgacagtgactgacagcaaaaatgtctttttccaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 41 - DNA, 1011 bp, Artificial Sequence
  Synthetic pACU14
  pACU14
gctcagcatctgcttcttcccaaagatgaacgcggcgttatgtcactaacgacgtgcaccaacttgcgggaattggaaaaagacatttttgctgtcagtcactgtcaagagattcttttgctggcatttcttccagaagcaaaaagagcgatgcgtcttttccgctgaaccgttccagcaaaaaagactaccaacgaattcgaaaaagacatttttgctgtcagtcactgtcaagagattcttttgctggcatttcttccagaagcaaaaagagcgatgcgtcttttccgctgaaccgttccagcaaaaaagactaccaacgaattcgaaaaagacatttttgctgtcagtcactgtcaagagattcttttgctggcatttcttccagaagcaaaaagagcgatgcgtcttttccgctgaaccgttccagcaaaaaagactaccaacgaattcgaaaaagacatttttgctgtcagtcactgtcaagagattcttttgctggcatttcttccagaagcaaaaagagcgatgcgtcttttccgctgaaccgttccagcaaaaaagactaccaaccaattccaccgcacgccttttttctgaagcccactttcgtggactttgccatatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 42 - DNA, 398 bp, Artificial Sequence
  Synthetic pACU15
  pACU15
tatagttttttctccttgacgttaaagtatagaggtatattaacaattttttgttgatacttttatgacatttgaataagaagtaatacaaactgaaaatgttgaaagtattagttaaagtggttatgcagcttttccatttatatatctgttaatagatcaaaaatcatcgcttcgctgattaattaccccagaaataaggctaaaaaactaatcgcattatcatccgaattcgttggtagtcttttttgctggaacggttcagcggaaaagacgcatcgctctttttgcttctggaagaaatgccagcaaaagaatctcttgacagtgactgacagcaaaaatgtctttttcgaattcgggctctttacatttccacaacatataagtaagattag

SEQ ID NO: 43 - DNA, 428 bp, Artificial Sequence
  Synthetic pGAL/CUP1p
  pGAL/CUP1p
ttatattgaattttcaaaaattcttactttttttttggatggacgcaaagaagtttaataatcatattacatggcattaccaccatatacatatccatatctaatcttacttatatgttgtggaaatgtaaagagcccgaattcgaaaaagacatttttgctgtcagtcactgtcaagagattcttttgctggcatttcttccagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 44 - DNA, 518 bp, Saccharomyces cerevisiae
  pCRS5
  pCRS5
gtggacgaaaagacataactgcagaagtacagctgcctttatttcttgtggtcatttattgcttttattttcaagtcagatatacaagaaaatcaaatcccatcgtcaacgtcacgtataaacgattaatttacagtaataccatactctaccaacattattttagtccgacgttcagtcctgtaggtgttccaaatccttctggcattgacttctgtgcagaaacccttcaaaatgagttccactttacgtcagatcgcataacaaccggtcatatatttttttcttttgctaaaccccctactgcaagcacttttaagaaaaagaacaataaatgcgtctttattgctgtgtggaagtgatttttgtctttcggacaaaaaaaggatagggatgcgagagggctgtgaagtagtgatcaagcggggcctatataagaagggcgcacatcgtcccccctaagaatagcgaagcgatattacactgaacactacaatgtcaaatagtactcaataaat

SEQ ID NO: 45 - DNA, 510 bp, Saccharomyces cerevisiae
  pCHA1
  pCHA1
gatctctgctgacgttgtatccacagatctaattgcaagatagcctcttgcgaccttattaaaagcctctccgtgatatcctctagggcttgggttgccattaatcgatgtgtccttgtttccttatgcgagctgtttcttatctatcttatggtcccattctttactgcactgtttacattttgatcaattgcgaaatgttcctactatttttctttttctcttttcgcgagtactaatcaccgcgaacggaaactaatgagtcctctgcgcggagacatgattccgcatgggcggctcctgttaagccccagcggaaatgtaattccactgagtgtcattaaatagtgccaaagctttatcaaattgtttgcgatgagataagataaaagggacaatatgaggaggaacacaggtatataaatatcgccaaataaaaggaaaatgtttatacagttttctcttttttaagtgctggatagacaagagacaggaaaattaaccagcgag

SEQ ID NO: 46 - DNA, 601 bp, Saccharomyces cerevisiae
  pCTR1
  pCTR1
caagtccgattgttcctcttcaggagcttcctgaaccaaactttttccgcaaggccgcattttgaaccgtattttgctcgttccagcctttccacgtttttgttatctaagcaacttggcacatttccctactatactacaaaccgatacgtaaatacttccctaaatagcatatgaattattcagtaatttttaaggatcgaaactgcacctcaactattcgttactgtggttatgttctcatgtattgatgcaaatcatgggatatttgctcaagacgacggtaaaatgagcaaaaatggcacgatcctgaaaagagcacttttcaagattcgggctacaaaatgcaacataaaaaatgttgtattgtcatctcgacagggtcttgtatgttttattcctcttatgattagttcacattagtaaaacagatacgcagtgtgctcttaataaacaactactccatagctttatttgcataacaaaacttttaagcacaaacttaaacaggtggagtaatagttcggcggcgactcaaattacatttgttggaagaatcgaatagaaaataaaaaaaagtgtattatatttgacattcaaa

SEQ ID NO: 47 - DNA, 522 bp, Saccharomyces cerevisiae
  pCTR3
  pCTR3
gatgtgatgacaaaacctcttccgataaaaacatttaaactattaacaaacaaatggattcattagatctattacattatgggtggtatgttggaataaaaatcaactatcatctactaactagtatttacgttactagtatattatcatatacggtgttagaagatgacgcaaatgatgagaaatagtcatctaaattagtggaagctgaaacgcaaggattgataatgtaataggatcaatgaatattaacatataaaacgatgataataatatttatagaattgtgtagaattgcagattcccttttatggattcctaaatcctccaggagaacttctagtatatctacatacctaatattattgccttattaaaaatggaatcccaacaattacatcaaaatccacattctcttcacttctccgatagacttgtaatttatcttatttcatttcctaacactttgatcgaagaagagggataacaacagacgaaaacacatttaagggctatacaaag

SEQ ID NO: 48 - DNA, 675 bp, Artificial Sequence
  Synthetic pCUR1
  pCUR1
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattgtcatgggatatttgctcaagacgacggtaaaatgagcaaatatggcacgatcctcaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 49 - DNA, 674 bp, Artificial Sequence
  Synthetic pCUR2
  pCUR2
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgattgaggatcgtgccatatttgctcattttaccgtcgtcttgagcaaatatcccatgacaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 50 - DNA, 794 bp, Artificial Sequence
  Synthetic pCUR3
  pCUR3
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattaggatcgtgccatatttgctcattttaccgtcgtcttgagcaaatatcccatgacaattgaggatcgtgccatatttgctcattttaccgtcgtcttgagcaaatatcccatgacaattgaggatcgtgccatatttgctcattttaccgtcgtcttgagcaaatatcccatgacaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 51 - DNA, 850 bp, Artificial Sequence
  Synthetic pCUR4
  pCUR4
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattgtcatgggatatttgctcaagacgacggtaaaatgagcaaatatggcacgatcctcaattgtcatgggatatttgctcaagacgacggtaaaatgagcaaatatggcacgatcctcaatgtcatgggatatttgctcaagacgacggtaaaatgagcaaatatggcacgatcctcaattgtcatgggatatttgctcaagacgacggtaaaatgagcaaatatcccatgacaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 52 - DNA, 491 bp, Artificial Sequence
  Synthetic pCUR5p
  pCUR5p
ttatattgaattttcaaaaattcttactttttttttggatggacgcaaagaagtttaataatcatattacatggcattaccaccatatacatatccatatctaatcttacttatatgttgtggaaatgtaaagagcccgaattgtcatgggatatttgctcaagacgacggtaaaatgagcaaatatggcacgatcctcaattgtcatgggatatttgctcaagacgacggtaaaatgagcaaatatggcacgatcccaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 53 - DNA, 833 bp, Artificial Sequence
  Synthetic pCUR6
  pCUR6
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccgaattgaggatcgtgccatatttgctcattttaccgtcgtcttgagcaaatatcccatgacaattgaggatcgtgccatatttgctcattttaccgtcgtcttgagcaaatatcccatgacaattgaggatcgtgccatatttgctcattttaccgtcgtcttgagcaaatatcccatgacaattcatgatcgcaaaatggcaaatggcacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 54 - DNA, 803 bp, Artificial Sequence
  Synthetic pCUR7
  pCUR7
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaattcaggatcgtgccatatttgctcattttaccgtcgtcttgagcaaatatcccatgacaattgaggatcgtgccatatttgctcattttaccgtcgtcttgagcaaatatcccatgagaattcttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 55 - DNA, 863 bp, Artificial Sequence
  Synthetic pCUR8
  pCUR8
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaattcaggatcgtgccatatttgctcattttaccgtcgtcttgagcaaatatcccatgacaattgaggatcgtgccatatttgctcattttaccgtcgtcttgagcaaatatcccatgacaattgaggatcgtgccatatttgctcattttaccgtcgtcttgagcaaatatcccatgagaattcttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 56 - DNA, 863 bp, Artificial Sequence
  Synthetic pCUR9
  pCUR9
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaattctcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctgaattcttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 57 - DNA, 923 bp, Artificial Sequence
  Synthetic pCUR10
  pCUR10
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaattctcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctgaattcttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 58 - DNA, 983 bp, Artificial Sequence
  Synthetic pCUR11
  pCUR11
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaattctcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctgaattcttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 59 - DNA, 1043 bp, Artificial Sequence
  Synthetic pCUR12
  pCUR12
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaattcaggatcgtgccatatttgctcattttaccgtcgtcttgagcaaatatcccatgacaattgaggatcgtgccatatttgctcattttaccgtcgtcttgagcaaatatcccatgacaattgaggatcgtgccatatttgctcattttaccgtcgtcttgagcaaatatcccatgagaattcaggatcgtgccatatttgctcattttaccgtcgtcttgagcaaatatcccatgacaattgaggatcgtgccatatttgctcattttaccgtcgtcttgagcaaatatcccatgacaattgaggatcgtgccatatttgctcattttaccgtcgtcttgagcaaatatcccatgagaattcttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 60 - DNA, 1043 bp, Artificial Sequence
  Synthetic pCUR13
  pCUR13
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaattctcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctgaattctcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctgaattcttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 61 - DNA, 1223 bp, Artificial Sequence
  Synthetic pCUR14
  pCUR14
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaattctcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctgaattctcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctgaattctcatgggatatttgctcaagacgacggtaaaatgagcaaatatggcacgatcctcaattgtcatgggatatttgctcaagacgacggtaaaatgagcaaatatggcacgatcctcaattgtcatgggatatttgctcaagacgacggtaaaatgagcaaatatggcacgatcctgaattcttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 62 - DNA, 1283 bp, Artificial Sequence
  Synthetic pCUR15
  pCUR15
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaattcaggatcgtgccatatttgctcattttaccgtcgtcttgagcaaatatcccatgacaattgaggatcgtgccatatttgctcattttaccgtcgtcttgagcaaatatcccatgacaattgaggatcgtgccatatttgctcattttaccgtcgtcttgagcaaatatcccatgacaattgaggatcgtgccatatttgctcattttaccgtcgtcttgagcaaatatcccatgagaattcaggatcgtgccatatttgctcattttaccgtcgtcttgagcaaatatcccatgacaattgaggatcgtgccatatttgctcattttaccgtcgtcttgagcaaatatcccatgacaattgaggatcgtgccatatttgctcattttaccgtcgtcttgagcaaatatcccatgagaattcaggatcgtgccatatttgctcattttaccgtcgtcttgagcaaatatcccatgacaattgaggatcgtgccatatttgctcattttaccgtcgtcttgagcaaatatcccatgacaattgaggatcgtgccatatttgctcattttaccgtcgtcttgagcaaatatcccatgagaattcttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 63 - DNA, 686 bp, Artificial Sequence
  Synthetic pCUR16
  pCUR16
gctcagcatctgcttcttcccaaagatgaacgcggcgttatgtcactaacgacgtgcaccaacttgcgggaattctcatgggatatttgctcaagacgacggtaaaatgagcaaatatggcacgatcctcaattgtcatgggatatttgctcaagacgacggtaaaatgagcaaatatggcacgatcctcaatgtcatgggatatttgctcaagacgacggtaaaatgagcaaatatggcacgatcctgaattccaccgcacgccttttttctgaagcccactttcgtggactttgccatatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 64 - DNA, 747 bp, Artificial Sequence
  Synthetic pCUR17
  pCUR17
gctcagcatctgcttcttcccaaagatgaacgcggcgttatgtcactaacgacgtgcaccaacttgcgggaattctcatgggatatttgctcaagacgacggtaaaatgagcaaatatggcacgatcctcaattgtcatgggatatttgctcaagacgacggtaaaatgagcaaatatggcacgatcctcaattctcatgggatatttgctcaagacgacggtaaaatgagcaaatatggcacgatcctcaattctcatgggatatttgctcaagacgacggtaaaatgagcaaatatggcacgatcctgaattccaccgcacgccttttttctgaagcccactttcgtggactttgccatatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 65 - DNA, 500 bp, Saccharomyces cerevisiae
  pLYS1
  pLYS1
gcaagttaacattagggagaacgtggggccttcctccatgagtgcagagcaattgaagatgtttagaggtttaaaggagaataaccagttgctggatagctctgtgccagctacagtttatgccaaattggcccttcatggtattcctgacggtgttaatggacagtacttgagctataatgaccctgccttggcggactttatgccttgaggatagcaggtacatataaattgttacatactaagtcgatgagtcaaaaaagactcttatacatttatacattttgcattattattttttttttccagcggaatttggaattccgctctcaaccgccaaaattcccctgcgatttcagcgacaaagagtcataaagtcatcctcgagaaaccacgatgaaatatataaaaagcccatcttccctgacggaaactggtattttaggaggcataccataagataacaacgaaaacgctttatttttcacacaaccgcaaaa

SEQ ID NO: 66 - DNA, 650 bp, Saccharomyces cerevisiae
  pLYS4
  pLYS4
ttgaaaaatgcgaagttgaagtgccatagaagagaaacagcccacacaggggagaagcccactggaaagggggcactgaccaactttaaataggaaacagaagataccacaagccagcgatacaacagcaccaaacaccgaaaagaatagccaaagctgtcctctggtgttggaaaaactggaaaaaacgcaactgcgttggctgctacggtgaaaaattttcctatgacttttttcactgcttgttcgtgcgaaattaccgcaaacccggtaaaatgtacacgtatcaagtgataaacaatttcgtgtcaagtgagcagaatggagcgatttggaaaaaaaaaatttttattgttttttcccccgggattttgctcgagatgactgaaattttgtaatcgatgagtctataccagaggcagcaaatatcaccaacatacacaggtatacacaatctcatgtccacacacacgtacagacacgcacatatatatatatatatatatatccccataggtatttatatatacaaaagaatcctcgtgtgtttgtgtgtgcaatagctagttttgcgctgcctcttatagtagacaatatcactttttcaataaaatagaacttgcaaggaaacaaaattgtatcgcttcaag

SEQ ID NO: 67 - DNA, 500 bp, Saccharomyces cerevisiae
  pLYS9
  pLYS9
acatatgcaagagtcttatgtatcgtatctaagtgccacgtaggggattcccatcatttgatgatttccaaatataatacctgtagagagcggtggagcaaaagtcaaattttaatcgcaactgcagacaagtcaagctgaggaaattgtggatgatctcttgtttcttttgatattcaccacaacagaagtgaagagtgtgattgcggttactactgaccacgaagcaatgcgtttagtagtgaaaagaattactcatactctggaatcgaaattccgttggaaaaattcgctttgtagtgaaaaataaagatgtcaataaagggtattgagaatttccaatggaattatcagcaatagatgatagaaagtagcacagaatttggcttaatggtatataaaccgtagggtcctggtaaaattacatgggaaggatccttaggcagtagggaaaacttatcaggacaattgagttatattaacgtattatatattttaat

SEQ ID NO: 68 - DNA, 494 bp, Artificial Sequence
  Synthetic pLYR1p
  pLYR1p
ttatattgaattttcaaaaattcttactttttttttggatggacgcaaagaagtttaataatcatattacatggcattaccaccatatacatatccatatctaatcttacttatatgttgtggaaatgtaaagagcccgaattcctcatactctggaatcgaaattccgttggaaaaattcgctttgtagtgaaaaataaagatgtcaataaagggtattgagaatttccaatggaattatcagcaatagatgatagaaagaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 69 - DNA, 494 bp, Artificial Sequence
  Synthetic pLYR2p
  pLYR2p
ttatattgaattttcaaaaattcttactttttttttggatggacgcaaagaagtttaataatcatattacatggcattaccaccatatacatatccatatctaatcttacttatatgttgtggaaatgtaaagagcccgaattctttctatcatctattgctgataattccattggaaattctcaataccctttattgacatctttatttttcactacaaagcgaatttttccaacggaatttcgattccagagtatgaggaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 70 - DNA, 616 bp, Artificial Sequence
  Synthetic pLYR3p
  pLYR3p
ttatattgaattttcaaaaattcttactttttttttggatggacgcaaagaagtttaataatcatattacatggcattaccaccatatacatatccatatctaatcttacttatatgttgtggaaatgtaaagagcccgaattcctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaagaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 71 - DNA, 616 bp, Artificial Sequence
  Synthetic pLYR4p
  pLYR4p
ttatattgaattttcaaaaattcttactttttttttggatggacgcaaagaagtttaataatcatattacatggcattaccaccatatacatatccatatctaatcttacttatatgttgtggaaatgtaaagagcccgaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 72 - DNA, 738 bp, Artificial Sequence
  Synthetic pLYR5p
  pLYR5p
ttatattgaattttcaaaaattcttactttttttttggatggacgcaaagaagtttaataatcatattacatggcattaccaccatatacatatccatatctaatcttacttatatgttgtggaaatgtaaagagcccgaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 73 - DNA, 1104 bp, Artificial Sequence
  Synthetic pLYR6p
  pLYR6p
ttatattgaattttcaaaaattcttactttttttttggatggacgcaaagaagtttaataatcatattacatggcattaccaccatatacatatccatatctaatcttacttatatgttgtggaaatgtaaagagcccgaattgctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaacaattgctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaacaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 74 - DNA, 1836 bp, Artificial Sequence
  Synthetic pLYR7p
  pLYR7p
ttatattgaattttcaaaaattcttactttttttttggatggacgcaaagaagtttaataatcatattacatggcattaccaccatatacatatccatatctaatcttacttatatgttgtggaaatgtaaagagcccgaattgtttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgagcaattgtttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgagcaattgtttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgagcaattgtttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgagcaattcggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 75 - DNA, 981 bp, Artificial Sequence
  Synthetic pLYR8
  pLYR8
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattgctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaacaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 76 - DNA, 981 bp, Artificial Sequence
  Synthetic pLYR9
  pLYR9
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattgtttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgagcaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 77 - DNA, 1225 bp, Artificial Sequence
  Synthetic pLYR10
  pLYR10
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattgtttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgaggaattctttctatcatctattgctgataattccattggaaattctcaataccctttattgacatctttatttttcactacaaagcgaatttttccaacggaatttcgattccagagtatgagcaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 78 - DNA, 1347 bp, Artificial Sequence
  Synthetic pLYR11
  pLYR11
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaattgctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaagaattcctcatactctggaatcgaaattccgttggaaaaattcgctttgtagtgaaaaataaagatgtcaataaagggtattgagaatttccaatggaattatcagcaatagatgatagaaacaattctaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 79 - DNA, 686 bp, Saccharomyces cerevisiae
  pMET17
  pMET17
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaggtcacatgatcgcaaaatggcaaatggcacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 80 - DNA, 510 bp, Saccharomyces cerevisiae
  pMET6
  pMET6
ccacaggaaatatttcacgtgacttacaaacagagtcgtacgtcaggaccggagtcaggtgaaaaaatgtgggccggtaaagggaaaaaaccagaaacgggactactatcgaactcgtttagtcgcgaacgtgcaaaaggccaatatttttcgctagagtcatcgcagtcatggcagctctttcgctctatctcccggtcgcaaaactgtggtagtcatagctcgttctgctcaattgagaactgtgaatgtgaatatggaacaaatgcgatagatgcactaatttaagggaagctagctagttttcccaactgcgaaagaaaaaaaggaaagaaaaaaaaattctatataagtgatagatatttccatctttactagcattagtttctcttttacgtattcaatatttttgttaaactcttcctttatcataaaaaagcaagcatctaagagcattgacaacactctaagaaacaaaataccaatataatttcaaagtacatatcaaaa

SEQ ID NO: 81 - DNA, 508 bp, Saccharomyces cerevisiae
  pMET14
  pMET14
cctatgcatgtttagagcaagcgcctttgtgagccctcccggttacgacgccttggcaatgtagcagataactctgcacttctagaatcattccactacgacatttggctcatcaccagctcgcgagaaatgtaaataagccaacaaccaagaatgcgtaacattaaagaatacagttgctttcatttcggcgtgatggtacggcacccacggttccttacattattctcgaaaaatagctgcacgcttttccaggaataaaagaccgtgccactaatttcacgtgatcaatatatttacaagccacctcaaaaaatgtggcaatggagaagaggatgaacgactcaatatgacttcaacttcatgaatttgtcaaaatatctatataagatgcaaaatttctatacaacatcagttgcgtatccgttaatgtcgttcattttctctctttgttcgaacttgacatcaagaaaagttggaattatttctccaagcacactgtacacca

SEQ ID NO: 82 - DNA, 552 bp, Saccharomyces cerevisiae
  pMET3
  pMET3
aacgatatgtacgtagtggtataaggtgagggggtccacagatataacatcgtttaatttagtactaacagagacttttgtcacaactacatataagtgtacaaatatagtacagatatgacacacttgtagcgccaacgcgcatcctacggattgctgacagaaaaaaaggtcacgtgaccagaaaagtcacgtgtaattttgtaactcaccgcattctagcggtccctgtcgtgcacactgcactcaacaccataaaccttagcaacctccaaaggaaatcaccgtataacaaagccacagttttacaacttagtctcttatgaagttacttaccaatgagaaatagaggctctttctcgacaaatatgaatatggatatatatatatatatatatatatatatatatatatatatgtaaacttggttcttttttagcttgtgatctctagcttgggtctctctctgtcgtaacagttgtgatatcgtttcttaacaattgaaaaggaactaagaaagtataataataacaagaataaagtataattaac

SEQ ID NO: 83 - DNA, 363 bp, Saccharomyces cerevisiae
  pSAM1
  pSAM1
gaaacggacgtaagacggaaatagaatttgaagataaagttatatatcactacacacgaatactttcttttttttttttcacaggaaaactgtggtggcgcccttgcctactagtgcatttcttttttcgggttcttgtctcgacgaaattttagcctcatcgtagtttttcactctggtatcgatgaaaaagggaagagtaaaaagttttccgtttagtacttaatgggattggtttgggacgtatatatcgactggtgttgtctgttattcatcgttgtttttcggttagcttcgaaaaaaaaatagagtaaaaaccaggaatttaccctaaaaacaagaaaaaataagataaacgaaaat

SEQ ID NO: 84 - DNA, 500 bp, Saccharomyces cerevisiae
  pSAM2
  pSAM2
gagctttgctctattatataagataaaatatgcactaaaagtttgcatttctttacataactaaaactaagacattatgcatagcttacctgatcaaaaagtatgtaaacttgttaacatcttcacatgtgattcatctggtcgtactttcttgcggtgcagtgtaatatttctacccacgtgactataattgagcttgaaaactgtggcgtttttccaccgatgggtccacgccagatattaaccgaagccaaaataccgatgaaatttctgagatagctcttgtaaacgacgtcaaatcttcatatgcaaggagatcttgatttctttttggtagtcatctgtcgtcttgaggcgtataagaaggaggttatatctgtcctttctacaaagtattttcgagaatcttgcttctgccccttttttctttttttaaaaggtttaaaaaacataactgtcttcaatatatccagtatttacgacaatatacaaacataatc

SEQ ID NO: 85 - DNA, 913 bp, Saccharomyces cerevisiae
  pMDH2
  pMDH2
ccttcgctaaataataaacctgaactgtacttagcgaagccttcatagcacctacgtacacgtatatatagacattttacgtaatggagaaactgaggtttttgttttcactttttttctttctttttcactattgctcgaaccgcctgcgatgagctaagaaaaaaaagtgaaagaaatcatagaaagcaaaaatgagattatatagcccagagccctcttctggcgcctgtcccaaggcggaccaacaacaacacttgcccaaacctaagaaaatcccctcatacttttccgtttgtatctcctactttcttacttcctttttttcttctttatttgcttggtttaccattgaagtccatttttactacagacaatagctagtcattcgctatcttccgtttgtcactttttttcaaatttctcatctatatagcgaagtacggaaaagatgtcacttgccggcatctcggccttccccggccaaatggactcatcatctacgatacggcccctttaatccgcaattactttgcccattcggccgtagccgttctaaagccgccgtgccttgcccccaatactcccctaatgatccgggaagttccggtttttttcctttgtttagtggcattttgtgttgcccaaggttgggaaggtccgatttgactttaaggaactacggaaggtatctaaggtttctaaaaacaatatacacgcgcgtgcgtagatatataaagataaagatttatcgatatgagataaagattgctgcatgattctccttctgattctttttccctgtatatattttctccccttctgtataaatcgtacagtcagaagtagtccagaatatagtgctgcagactattacaaaagttcaatacaatatcataaaagttatagtaac

SEQ ID NO: 86 - DNA, 998 bp, Saccharomyces cerevisiae
  pJEN1
  pJEN1
aatgtgtttataaattattttttttgctggtagcaaaatcaactcattgtcttccattcagagtctaatcgaacgttatcgcaatgcttgcacacttttaaacaatacgatttagtttaagtggatggacccccacgcttagtgttccacaggtttgtccccactgtttttacattccactgtacatttttgcaatagaaggtcattgtatgctaccttgggcggctaagaatacctgtaaaaatttggagaaattagattcgtaaagaatgactcgcaacgactccaatgatttcttcttttcaccctttgaacggccgatatccgcgcgggatcctgaccccgcaatttactccactagaccggcgtgtttctctttttccttttcctggggttagagcccaagagctaatagccgacaaacggactccaaaaaaaaaaggaggcacaggacaaacgcagcacctgcgtcattcacgctgaagcggcagcaagcattttcgatcagctccaattaaatgaagactattcgccgtaccgttcccagatgggtgcgaaagtcagtgatcgaggaagttattgagcgcgcggcttgaaactatttctccatctcagagccgccaagcctaccattattctccaccaggaagttagtttgtaagcttctgcacaccatccggacgtccataattcttcacttaacggtcttttgcccccccttctactataatgcattagaacgttacctggtcatttggatggagatctaagtaacacttactatctcctatggtactatcctttaccaaaaaaaaaaaaaaaaaaaaaaaaaaaaatcagcaaagtgaagtaccctcttgatgtataaatacattgcacatcattgttgagaaatagttttggaagttgtctagtccttctcccttagatctaaaaggaagaagagtaacagtttcaaaagtttttcctcaaagagattaaatactgctactgaaaat

SEQ ID NO: 87 - DNA, 450 bp, Saccharomyces cerevisiae
  pICL1
  pICL1
ttttgctactcgtcatccgatgagaaaaactgttcccttttgccccaggtttccattcatccgagcgatcacttatctgacttcgtcactttttcatttcatccgaaacaatcaaaactgaagccaatcaccacaaaattaacactcaacgtcatctttcactaccctttacagaagaaaatatccatagtccggactagcatcccagtatgtgactcaatattggtgcaaaagagaaaagcataagtcagtccaaagtccgcccttaaccaggcacatcggaattcacaaaacgtttctttattatataaaggagctgcttcactggcaaaattcttattatttgtcttggcttgctaatttcatcttatcctttttttcttttcacacccaaatacctaacaattgagagaaaactcttagcataacataacaaaaagtcaacgaaaa

SEQ ID NO: 88 - DNA, 598 bp, Saccharomyces cerevisiae
  pADH2
  pADH2
tatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacaca

SEQ ID NO: 89 - DNA, 793 bp, Saccharomyces cerevisiae
  pMLS1
  pMLS1
tgtctaatgcgaaggtacttttatttttttcagattcaaagcaatattatttagacaattgatactaagtgagcttaaggaggattaaacaactgtggaatccttcacaaggattcaatatttgtttttcctggttattttgccatcattcaactttcctcagacgtaaaattcgtgcttagtgatgtctcaatattcccgcagggtaataaaattcaataactatcactatatacgcaacagtattaccctacattgctatcggctcaatggaaatccccatatcatagcttccattgggccgatgaagttagtcgacggatagaagcggttgtcccctttcccggcgagccggcagtcgggccgaggttcggataaattttgtattgtgttttgattctgtcatgagtattacttatgttctctttaggtaaccccaggttaatcaatcacagtttcataccggctagtattcaaattatgacttttcttctgcagtgtcagccttacgacgattatctatgagctttgaatatagtttgccgtgattcgtatctttaattggataataaaatgcgaaggatcgatgacccttattattatttttctacactggctaccgatttaactcatcttcttgaaagtatataagtaacagtaaaatataccgtacttctgctaatgttatttgtcccttatttttcttttcttgtcttatgctatagtacctaagaataacgactattgttttgaactaaacaaagtagtaaaagcacataaaagaattaagaaa

SEQ ID NO: 90 - DNA, 300 bp, Saccharomyces cerevisiae
  tTDH2
  tTDH2
atttaactccttaagttactttaatgatttagtttttattattaataattcatgctcatgacatctcatatacacgtttataaaacttaaatagattgaaaatgtattaaagattcctcagggattcgatttttttggaagtttttgtttttttttccttgagatgctgtagtatttgggaacaattatacaatcgaaagatatatgcttacattcgaccgttttagccgtgatcattatcctatagtaacataacctgaagcataactgacactactatcatcaatacttgtcacatga

SEQ ID NO: 91 - DNA, 300 bp, Saccharomyces cerevisiae
  tCYC1
  tCYC1
acaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagcttcgcagtttacactctcatc

SEQ ID NO: 92 - DNA, 300 bp, Saccharomyces cerevisiae
  tTDH3
  tTDH3
gtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggat

SEQ ID NO: 93 - DNA, 354 bp, Saccharomyces cerevisiae
  tADH1
  tADH1
actagttctagagcggccgccaccgcggtgggcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttcca

SEQ ID NO: 94 - DNA, 301 bp, Saccharomyces cerevisiae
  tADH2
  tADH2
gcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcatt

SEQ ID NO: 95 - DNA, 299 bp, Saccharomyces cerevisiae
  tTPI1
  tTPI1
gattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattactttgtctctaaatccttgtaaaatgtgtacgatctctatatgggttactc

SEQ ID NO: 96 - DNA, 299 bp, Saccharomyces cerevisiae
  tMET17
  tMET17
gtgtgcgtaatgagttgtaaaattatgtataaacctactttctctcacaagtactatacttttataaaacgaactttattgaaatgaatatcctttttttcccttgttacatgtcgtgactcgtactttgaacctaaattgttctaacatcaaagaacagtgttaattcgcagtcgagaagaaaaatatggtgaacaagactcatctacttcatgagactactttacgcctcctataaagctgtcacactggataaatttattgtaggaccaagttacaaaagaggatgatggaggttt

SEQ ID NO: 97 - DNA, 305 bp, Saccharomyces cerevisiae
  tENO2
  tENO2
ggatcctaaagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaataactacatggatgataagaaaacatggagtacagtcactttgagaaccttcaatcagctggtaacgtcttc

SEQ ID NO: 98 - DNA, 300 bp, Saccharomyces cerevisiae
  tMET3
  tMET3
tcgtcataaaatgctcccatctcaaaagtagggcaaaattcatgatcgaccgcgcaaaataaatagatttgcaaataagttttgtatgtacatttattaatatatataatatatcaaaagaaaaaaatcaaaaaaaaaaaaaaaaaaaaattgcactcttattcagtcatcaattacaaaacctagagatagcgatggtgcatattcaataaaaaactccttatactgtcgagaaagcttattattggtacttctcgaagatactaaaaaaggttaatttttggagacggaggcaatagc

SEQ ID NO: 99 - DNA, 301 bp, Saccharomyces cerevisiae
  tPGK1
  tPGK1
attgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagctttcgagaaggatattattta

SEQ ID NO: 100 - DNA, 300 bp, Saccharomyces cerevisiae
  tDIT1
  tDIT1
taaagtaagagcgctacattggtctacctttttgttcttttacttaaacattagttagttcgttttctttttctcatttttttatgtttcccccccaaagttctgattttataatattttatttcacacaattccatttaacagagggggaatagattctttagcttagaaaattagtgatcaatatatatttgcctttcttttcatcttttcagtgatattaatggtttcgagacactgcaatggccctagttgtctaagaggatagatgttactgtcaaagatgatattttgaatttc

SEQ ID NO: 101 - DNA, 300 bp, Saccharomyces cerevisiae
  tRPL3
  tRPL3
gaagttttgttagaaaataaatcattttttaattgagcattcttattcctattttatttaaatagttttatgtattgttagctacatacaacagtttaaatcaaattttctttttcccaagtccaaaatggaggtttattttgatgacccgcatgcgattatgttttgaaagtataagactacatacatgtacatatatttaaacatgtaaacccgtccattatattgcttactttcttcttttttgccgttttgacttggacctctggtttgctatttccttacaatctttgctacaat

SEQ ID NO: 102 - DNA, 300 bp, Saccharomyces cerevisiae
  tRPL41B
  tRPL41B
gcggattgagagcaaatcgttaagttcaggtcaagtaaaaattgatttcgaaaactaatttctcttatacaatcctttgattggaccgtcatcctttcgaatataagattttgttaagaatattttagacagagatctactttatatttaatatctagatattacataatttcctctctaataaaatatcattaataaaataaaaatgaagcgatttgattttgtgttgtcaacttagtttgccgctatgcctcttgggtaatgctattattgaatcgaagggctttattatattaccct

SEQ ID NO: 103 - DNA, 300 bp, Saccharomyces cerevisiae
  tRPL15A
  tRPL15A
gctggttgatggaaaatataattttattgggcaaacttttgtttatctgatgtgttttatactattatctttttaattaatgattctatatacaaacctgtatattttttctttaaccaatttttttttttatagacctagagctgtacttttattctgctatcaagcaaacccctaccccctcttctcaatcctcccctcaggcagaacttatctacctgtatcaaggagcggacgagggagtcctaattgttctacgtataccaatgctagcagcttacataggtggtggcactacca

SEQ ID NO: 104 - DNA, 300 bp, Saccharomyces cerevisiae
  tIDP1
  tIDP1
tcgaatttacgtagcccaatctaccacttttttttttcattttttaaagtgttatacttagttatgctctaggataatgaactacttttttttttttttttttactgttatcataaatatatataccttattgttgtttgcaaccgtcggttaattccttatcaaggttccccaagttcggatcattaccatcaatttccaacattttcatgagttcttcttcttcattaccgtgttttagggggctgttcgcacttctaatagggctatcaccaagctgttctaattcgtccaaaagtt

SEQ ID NO: 105 - protein, 528 aa, Artificial Sequence
  Synthetic amino acid sequence of the enzymes able to catalyze the decarboxylation of oxaloacetate into malonic semi-aldehyde
  VARIANT 19: Xaa in position 19 is selected from the group consisting of threonine and isoleucine
  VARIANT 109: Xaa in position 109 is selected from the group consisting of leucine, lysine, arginine and valine
  VARIANT 110: Xaa in position 110 is selected from the group consisting of leucine and lysine
  VARIANT 180: Xaa in position 180 is selected from the group consisting of serine and threonine
  VARIANT 204: Xaa in position 204 is selected from the group consisting of alanine and valine
  VARIANT 281: Xaa in position 281 is selected from the group consisting of histidine and arginine
  VARIANT 282: Xaa in position 282 is selected from the group consisting of glutamine and arginine
  VARIANT 369: Xaa in position 369 is selected from the group consisting of asparagine and aspartic acid
  VARIANT 377: Xaa in position 377 is selected from the group consisting of threonine and serine
  VARIANT 388: Xaa in position 388 is selected from the group consisting of asparagine and aspartic acid
  VARIANT 397: Xaa in position 397 is selected from the group consisting of phenylalanine, asparagine, alanine, isoleucine, valine, leucine, tryptophan and arginine
  VARIANT 398: Xaa in position 398 is selected from the group consisting of cysteine and arginine
  VARIANT 403: Xaa in position 403 is selected from the group consisting of leucine, asparagine, alanine, valine and serine
  VARIANT 419: Xaa in position 419 is selected from the group consisting of glutamic acid and glycine
  VARIANT 446: Xaa in position 446 is selected from the group consisting of isoleucine and valine
  VARIANT 460: Xaa in position 460 is selected from the group consisting of alanine, leucine, threonine, glycine and asparagine
  VARIANT 464: Xaa in position 464 is selected from the group consisting of phenylalanine and serine
  VARIANT 469: Xaa in position 469 is selected from the group consisting of glutamic acid and glycine
MASVHGTTYELLRRQGIDXVFGNPGSNELPFLKDFPEDFRYILALQEACVVGIADGYAQASRKPAFINLHSAAGTGNAMGALSNAWNSHSPLIVTAGQQTRAMIGVEAXXTNVDAANLPRPLVKWSYEPASAAEVPHAMSRAIHMASMAPQGPVYLSVPYDDWDKDADPQSHHLFDRHVXSSVRLNDQDLDILVKALNSASNPXIVLGPDVDAANANADCVMLAERLKAPVWVAPSAPRCPFPTRHPCFRGLMPAGIAAISQLLEGHDVVLVIGAPVFRYXXYDPGQYLKPGTRLISVTCDPLEAARAPMGDAIVADIGAMASALANLVEESSRQLPTAAPEPAKVDQDAGRLHPETVFDTLNDMAPEXAIYLNESXSTTAQMWQRLXMRNPGSYYXXAAGGXGFALPAAIGVQLAEPXRQVIAVIGDGSANYSISALWTAAQYNXPTIFVIMNNGTYGXLRWXAGVLXAENVPGLDVPGIDFRALAKGYGVQALKADNLEQLKGSLQEALSAKGPVLIEVSTVSPVK

SEQ ID NO: 106 - protein, 528 aa, Artificial Sequence
  Synthetic Enzyme 6
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYLCAAGGLGFALPAAIGVQLAEPERQVIAVIGDGSANYSISALWTAAQYNVPTIFVIMNNGTYGTLRWFAGVLEAENVPGLDVPGIDFRALAKGYGVQALKADNLEQLKGSLQEALSAKGPVLIEVSTVSPVK

SEQ ID NO: 107 - protein, 528 aa, Artificial Sequence
  Synthetic Enzyme 7
MASVHGTTYELLRRQGIDIVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYRQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLDMRNPGSYYFCAAGGLGFALPAAIGVQLAEPGRQVIAVIGDGSANYSISALWTAAQYNIPTIFVIMNNGTYGLLRWSAGVLGAENVPGLDVPGIDFRALAKGYGVQALKADNLEQLKGSLQEALSAKGPVLIEVSTVSPVK

SEQ ID NO: 108 - protein, 528 aa, Artificial Sequence
  Synthetic Enzyme 8
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYRQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGGLRWFAGVLEAENVPGLDVPGIDFRALAKGYGVQALKADNLEQLKGSLQEALSAKGPVLIEVSTVSPVK

SEQ ID NO: 109 - protein, 528 aa, Artificial Sequence
  Synthetic Enzyme 9
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGTLRWFAGVLEAENVPGLDVPGIDFRALAKGYGVQALKADNLEQLKGSLQEALSAKGPVLIEVSTVSPVK

SEQ ID NO: 110 - protein, 528 aa, Artificial Sequence
  Synthetic Enzyme 10
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYACAAGGLGFALPAAIGVQLAEPERQVIAVIGDGSANYSISALWTAAQYNIPTIFVIMNNGTYGALRWFAGVLEAENVPGLDVPGIDFRALAKGYGVQALKADNLEQLKGSLQEALSAKGPVLIEVSTVSPVK

SEQ ID NO: 111 - protein, 528 aa, Artificial Sequence
  Synthetic Enzyme 11
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGLLRWFAGVLEAENVPGLDVPGIDFRALAKGYGVQALKADNLEQLKGSLQEALSAKGPVLIEVSTVSPVK

SEQ ID NO: 112 - protein, 528 aa, Artificial Sequence
  Synthetic Enzyme 12
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYRQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGALRWFAGVLEAENVPGLDVPGIDFRALAKGYGVQALKADNLEQLKGSLQEALSAKGPVLIEVSTVSPVK

SEQ ID NO: 113 - protein, 528 aa, Artificial Sequence
  Synthetic Enzyme 13
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYRQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGTLRWFAGVLEAENVPGLDVPGIDFRALAKGYGVQALKADNLEQLKGSLQEALSAKGPVLIEVSTVSPVK

SEQ ID NO: 114 - protein, 528 aa, Artificial Sequence
  Synthetic Enzyme 14
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGALRWFAGVLEAENVPGLDVPGIDFRALAKGYGVQALKADNLEQLKGSLQEALSAKGPVLIEVSTVSPVK

SEQ ID NO: 115 - protein, 528 aa, Artificial Sequence
  Synthetic Enzyme 15
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGLLRWFAGVLEAENVPGLDVPGIDFRALAKGYGVQALKADNLEQLKGSLQEALSAKGPVLIEVSTVSPVK

SEQ ID NO: 116 - protein, 528 aa, Artificial Sequence
  Synthetic Enzyme 116
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYLCAAGGLGFALPAAIGVQLAEPERQVIAVIGDGSANYSISALWTAAQYNVPTIFVIMNNGTYGLLRWFAGVLEAENVPGLDVPGIDFRALAKGYGVQALKADNLEQLKGSLQEALSAKGPVLIEVSTVSPVK

SEQ ID NO: 117 - protein, 528 aa, Artificial Sequence
  Synthetic Enzyme 17
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYLCAAGGLGFALPAAIGVQLAEPERQVIAVIGDGSANYSISALWTAAQYNVPTIFVIMNNGTYGGLRWFAGVLEAENVPGLDVPGIDFRALAKGYGVQALKADNLEQLKGSLQEALSAKGPVLIEVSTVSPVK

SEQ ID NO: 118 - protein, 528 aa, Artificial Sequence
  Synthetic Enzyme 18
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYLCAAGGLGFALPAAIGVQLAEPERQVIAVIGDGSANYSISALWTAAQYNVPTIFVIMNNGTYGTLRWSAGVLEAENVPGLDVPGIDFRALAKGYGVQALKADNLEQLKGSLQEALSAKGPVLIEVSTVSPVK

SEQ ID NO: 119 - protein, 528 aa, Artificial Sequence
  Synthetic Enzyme 19
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYLCAAGGLGFALPAAIGVQLAEPGRQVIAVIGDGSANYSISALWTAAQYNVPTIFVIMNNGTYGTLRWSAGVLGAENVPGLDVPGIDFRALAKGYGVQALKADNLEQLKGSLQEALSAKGPVLIEVSTVSPVK

SEQ ID NO: 120 - protein, 528 aa, Artificial Sequence
  Synthetic Enzyme 20
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYLCAAGGLGFALPAAIGVQLAEPGRQVIAVIGDGSANYSISALWTAAQYNIPTIFVIMNNGTYGTLRWSAGVLGAENVPGLDVPGIDFRALAKGYGVQALKADNLEQLKGSLQEALSAKGPVLIEVSTVSPVK

SEQ ID NO: 121 - protein, 528 aa, Artificial Sequence
  Synthetic Enzyme 21
MASVHGTTYELLRRQGIDIVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLDMRNPGSYYFCAAGGLGFALPAAIGVQLAEPGRQVIAVIGDGSANYSISALWTAAQYNIPTIFVIMNNGTYGLLRWSAGVLGAENVPGLDVPGIDFRALAKGYGVQALKADNLEQLKGSLQEALSAKGPVLIEVSTVSPVK

SEQ ID NO: 122 - protein, 528 aa, Artificial Sequence
  Synthetic Enzyme 22
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYRQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYNCAAGGLGFALPAAIGVQLAEPERQVIAVIGDGSANYSISALWTAAQYNIPTIFVIMNNGTYGLLRWFAGVLEAENVPGLDVPGIDFRALAKGYGVQALKADNLEQLKGSLQEALSAKGPVLIEVSTVSPVK

SEQ ID NO: 123 - protein, 528 aa, Artificial Sequence
  Synthetic Enzyme 23
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYRQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYWCAAGGLGFALPAAIGVQLAEPERQVIAVIGDGSANYSISALWTAAQYNIPTIFVIMNNGTYGALRWFAGVLEAENVPGLDVPGIDFRALAKGYGVQALKADNLEQLKGSLQEALSAKGPVLIEVSTVSPVK

SEQ ID NO: 124 - protein, 528 aa, Artificial Sequence
  Synthetic Enzyme 24
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYRQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYRCAAGGVGFALPAAIGVQLAEPERQVIAVIGDGSANYSISALWTAAQYNIPTIFVIMNNGTYGALRWFAGVLEAENVPGLDVPGIDFRALAKGYGVQALKADNLEQLKGSLQEALSAKGPVLIEVSTVSPVK

SEQ ID NO: 125 - protein, 528 aa, Artificial Sequence
  Synthetic Enzyme 25
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYRQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYWCAAGGLGFALPAAIGVQLAEPERQVIAVIGDGSANYSISALWTAAQYNIPTIFVIMNNGTYGNLRWFAGVLEAENVPGLDVPGIDFRALAKGYGVQALKADNLEQLKGSLQEALSAKGPVLIEVSTVSPVK

SEQ ID NO: 126 - protein, 528 aa, Artificial Sequence
  Synthetic Enzyme 26
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVTSSVRLNDQDLDILVKALNSASNPVIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGTLRWFAGVLEAENVPGLDVPGIDFRALAKGYGVQALKADNLEQLKGSLQEALSAKGPVLIEVSTVSPVK

SEQ ID NO: 127 - protein, 528 aa, Artificial Sequence
  Synthetic Enzyme 27
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYACAAGGLGFALPAAIGVQLAEPERQVIAVIGDGSANYSISALWTAAQYNIPTIFVIMNNGTYGTLRWFAGVLEAENVPGLDVPGIDFRALAKGYGVQALKADNLEQLKGSLQEALSAKGPVLIEVSTVSPVK

SEQ ID NO: 128 - protein, 528 aa, Artificial Sequence
  Synthetic Enzyme 28
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYLCAAGGLGFALPAAIGVQLAEPERQVIAVIGDGSANYSISALWTAAQYNIPTIFVIMNNGTYGTLRWFAGVLEAENVPGLDVPGIDFRALAKGYGVQALKADNLEQLKGSLQEALSAKGPVLIEVSTVSPVK

SEQ ID NO: 129 - protein, 528 aa, Artificial Sequence
  Synthetic Enzyme 29
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYRCAAGGVGFALPAAIGVQLAEPERQVIAVIGDGSANYSISALWTAAQYNVPTIFVIMNNGTYGALRWFAGVLEAENVPGLDVPGIDFRALAKGYGVQALKADNLEQLKGSLQEALSAKGPVLIEVSTVSPVK

SEQ ID NO: 130 - protein, 528 aa, Artificial Sequence
  Synthetic Enzyme 30
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYRCAAGGVGFALPAAIGVQLAEPERQVIAVIGDGSANYSISALWTAAQYNVPTIFVIMNNGTYGGLRWFAGVLEAENVPGLDVPGIDFRALAKGYGVQALKADNLEQLKGSLQEALSAKGPVLIEVSTVSPVK

SEQ ID NO: 131 - protein, 528 aa, Artificial Sequence
  Synthetic Enzyme 31
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYWCAAGGSGFALPAAIGVQLAEPERQVIAVIGDGSANYSISALWTAAQYNVPTIFVIMNNGTYGNLRWFAGVLEAENVPGLDVPGIDFRALAKGYGVQALKADNLEQLKGSLQEALSAKGPVLIEVSTVSPVK

SEQ ID NO: 132 - protein, 528 aa, Artificial Sequence
  Synthetic Enzyme 32
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYRQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYWCAAGGLGFALPAAIGVQLAEPERQVIAVIGDGSANYSISALWTAAQYNIPTIFVIMNNGTYGLLRWFAGVLEAENVPGLDVPGIDFRALAKGYGVQALKADNLEQLKGSLQEALSAKGPVLIEVSTVSPVK

SEQ ID NO: 133 - protein, 528 aa, Artificial Sequence
  Synthetic Enzyme 33
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYRQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYRCAAGGLGFALPAAIGVQLAEPERQVIAVIGDGSANYSISALWTAAQYNIPTIFVIMNNGTYGLLRWFAGVLEAENVPGLDVPGIDFRALAKGYGVQALKADNLEQLKGSLQEALSAKGPVLIEVSTVSPVK

SEQ ID NO: 134 - protein, 528 aa, Artificial Sequence
  Synthetic Enzyme 34
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYWCAAGGLGFALPAAIGVQLAEPERQVIAVIGDGSANYSISALWTAAQYNIPTIFVIMNNGTYGTLRWFAGVLEAENVPGLDVPGIDFRALAKGYGVQALKADNLEQLKGSLQEALSAKGPVLIEVSTVSPVK

SEQ ID NO: 135 - protein, 528 aa, Artificial Sequence
  Synthetic Enzyme 35
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYRCAAGGLGFALPAAIGVQLAEPERQVIAVIGDGSANYSISALWTAAQYNIPTIFVIMNNGTYGTLRWFAGVLEAENVPGLDVPGIDFRALAKGYGVQALKADNLEQLKGSLQEALSAKGPVLIEVSTVSPVK

SEQ ID NO: 136 - protein, 528 aa, Artificial Sequence
  Synthetic Enzyme 36
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYRCAAGGVGFALPAAIGVQLAEPERQVIAVIGDGSANYSISALWTAAQYNVPTIFVIMNNGTYGTLRWFAGVLEAENVPGLDVPGIDFRALAKGYGVQALKADNLEQLKGSLQEALSAKGPVLIEVSTVSPVK

SEQ ID NO: 137 - protein, 528 aa, Artificial Sequence
  Synthetic Enzyme 37
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEARLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHRYDPGQYLKPGTRLISVTCDPLEAARAPMGDAIVADIGAMASALANLVEESSRQLPTAAPEPAKVDQDAGRLHPETVFDTLNDMAPEDAIYLNESTSTTAQMWQRLNMRNPGSYYWCAAGGSGFALPAAIGVQLAEPERQVIAVIGDGSANYSISALWTAAQYNVPTIFVIMNNGTYGTLRWFAGVLEAENVPGLDVPGIDFRALAKGYGVQALKADNLEQLKGSLQEALSAKGPVLIEVSTVSPVK

(19) United States
(12) Patent Application Publication
(10) Pub. No.: US 2022/0049279 A1
(43) Pub. Date: Feb. 17, 2022

(54) MALONIC SEMI-ALDEHYDE-PRODUCING YEASTS

(71) Applicant: ALDERYS, Orsay (FR)

(72) Inventors: Dominique LOUIS, Orsay (FR); Karine JAILLARDON, Orsay (FR)

(21) Appl. No.: 17/414,604

(22) PCT Filed: Dec. 18, 2019
(86) PCT No.: PCT/EP2019/085986

§ 371 Date: Jun. 16, 2021

(30) Foreign Application Priority Data
Dec. 18, 2018 (EP) 18306717.2

(51) Int. Cl. C12P 7/40; C12N 1/18; C12N 9/88

(52) U.S. Cl. CPC C12P 7/40; C12N 9/88; C12N 1/18

(57) ABSTRACT

The present invention relates to a recombinant yeast, in the genome of which at least one nucleic acid encoding an enzyme able to catalyze the decarboxylation of oxaloacetate into malonic semi-aldehyde, or one of its salts is overexpressed and/or under the control of a strong or inducible promoter. The invention also relates to such an enzyme per se, as well as to a method for producing malonic semi-aldehyde, one of its salts, or a malonic semi-aldehyde derivative comprising implementing the said recombinant yeast and to the use of the said recombinant yeast for the production of malonic semi-aldehyde, one of its salts, or a malonic semi-aldehyde derivative.

### FIELD OF THE INVENTION

The present invention relates to the field of bio-production of malonic semi-aldehyde, one of its salts, or a malonic semi-aldehyde derivative, and in particular of bio-production of malonic semi-aldehyde, one of its salts, or a malonic semi-aldehyde derivative from oxaloacetate in a yeast.

### BACKGROUND OF THE INVENTION

Malonic semi-aldehyde and its salts thereof are a key intermediate for the production of valuable compounds. These compounds of economic interest include those produced directly from malonic semi-aldehyde or its salts, such as for example Acrylate, 1-propanol, isopropanol, 3-hydroxypropionate and propionate, but also those derived from malonyl-CoA, mostly produced by polyketides synthases such as phloroglucinol and flavonoids, and the fatty acids synthase, or those derived from the mevalonate such as farnesyl-PP, squalene and derivatives or the 3-hydroxy-3-methyl-butyrate pathways.

Malonic semi-aldehyde and its salts are naturally mainly produced in yeast from (malonyl-CoA) and beta-alanine. However, production of malonyl-coA and its salts is in competition with the ethanol biosynthesis pathway, thus rendering difficult the flux derivation to malonic semi-aldehyde.

Moreover, production from beta-alanine requires the amination of oxaloacetate followed by the deamination of beta alanine, involving a great number of enzymes.

In order to facilitate the production of malonic semi-aldehyde and its salts in yeasts, it has been proposed to obtain malonyl semi-aldehyde in one step by decarboxylation of oxaloacetate (US2010/0021978). However, no natural enzyme is known as being able to operate this transformation. The enzymatic activity of decarboxylation of oxaloacetate into malonic semi-aldehyde is herein referred to as oxaloacetate 1-decarboxylase (MSA forming), and is not to be confused with oxaloacetate decarboxylase (EC 4.1.1.3) which yields pyruvate.

US2010/0021978 proposes to use promiscuous decarboxylase such as the benzoylformate decarboxylase, the alpha-ketoglutarate decarboxylase, the alpha-ketoisovalerate decarboxylase or the pyruvate decarboxylase to perform this decarboxylation of oxaloacetate in malonic acid or one of its salts. This document exemplifies the use of benzoylformate decarboxylase in Escherichia coli to produce 3 hydroxypropionate through malonyl semi-aldehyde.

However, the inventors have expressed in the yeast Saccharomyces cerevisiae these different enzymes. While they could detect their carboxylase activity both in cellulo and in vitro on their cognate substrate, the inventors were unable to detect any activity of all these enzymes on oxaloacetate.

Accordingly, there is still a need in the art for enzymes able to efficiently catalyze the transformation of oxaloacetate into malonic semi-aldehyde, or one of its salts, when expressed in a yeast, and in particular in the yeast Saccharomyces cerevisiae.

### SUMMARY OF THE INVENTION

The present invention accordingly relates to a recombinant yeast, in the genome of which at least one nucleic acid encoding an enzyme able to catalyze the decarboxylation of oxaloacetate into malonic semi-aldehyde, or one of its salts, is:

(i) overexpressed and/or

(ii) under the control of a strong or inducible promoter;

said enzyme being of sequence SEQ ID NO:105:

(SEQ ID NO: 105)
MASVHGTTYELLRRQGIDX₈VFGNPGSNELPFLKDFPEDFRYILALQEAC
VVGIADGYAQASRKPAFINLHSAAGTGNAMGALSNAWNSHSPLIVTAGQQ
TRAMIGVEAX₁X₂TNVDAANLPRPLVKWSYEPASAAEVPHAMSRAIHMAS
MAPQGPVYLSVPYDDWDKDADPQSHHLFDRHVX₉SSVRLNDQDLDILVKA
LNSASNPX₁₀IVLGPDVDAANANADCVMLAERLKAPVWVAPSAPRCPFPT
RHPCFRGLMPAGIAAISQLLEGHDVVLVIGAPVFRYX₁₁X₁₂YDPGQYLK
PGTRLISVTCDPLEAARAPMGDAIVADIGAMASALANLVEESSRQLPTAA
PEPAKVDQDAGRLHPETVFDTLNDMAPEX₁₃AIYLNESX₃STTAQMWQRL
X₁₄MRNPGSYYX₄X₅AAGGX₆GFALPAAIGVQLAEPX₁₅RQVIAVIGDGS
ANYSISALWTAAQYNX₁₆PTIFVIMNNGTYGX₇LRWX₁₇AGVLX₁₈AENV
PGLDVPGIDFRALAKGYGVQALKADNLEQLKGSLQEALSAKGPVLIEVST
VSPVK

wherein:
X₁ represents an amino acid selected from the group consisting of leucine, lysine, arginine and valine;
X₂ represents an amino acid selected from the group consisting of leucine and lysine;
X₃ represents an amino acid selected from the group consisting of threonine and serine;
X₄ represents an amino acid selected from the group consisting of phenylalanine, asparagine, alanine, isoleucine, valine, leucine, tryptophan and arginine;
X₅ represents an amino acid selected from the group consisting of cysteine and arginine;
X₆ represents an amino acid selected from the group consisting of leucine, asparagine, alanine, valine and serine;
X₇ represents an amino acid selected from the group consisting of alanine, leucine, threonine, glycine and asparagine;
X₈ represents an amino acid selected from the group consisting of threonine and isoleucine;
X₉ represents an amino acid selected from the group consisting of serine and threonine;
X₁₀ represents an amino acid selected from the group consisting of alanine and valine;
X₁₁ represents an amino acid selected from the group consisting of histidine and arginine;
X₁₂ represents an amino acid selected from the group consisting of glutamine and arginine;
X₁₃ represents an amino acid selected from the group consisting of asparagine and aspartic acid;
X₁₄ represents an amino acid selected from the group consisting of asparagine and aspartic acid;
X₁₅ represents an amino acid selected from the group consisting of glutamic acid and glycine;
X₁₆ represents an amino acid selected from the group consisting of isoleucine and valine;
X₁₇ represents an amino acid selected from the group consisting of phenylalanine and serine; and
X₁₈ represents an amino acid selected from the group consisting of glutamic acid and glycine;
with the proviso that the enzyme cannot have the sequence SEQ ID NO: 105 wherein X₁ represents leucine; X₂ represents leucine; X₃ represents threonine; X₄ represents phenylalanine; X₅ represents cysteine; X₆ represents leucine; X₇ represents alanine, X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine and X₁₈ represents glutamic acid.

The present invention in particular relates to a recombinant yeast, in the genome of which at least one nucleic acid encoding an enzyme able to catalyze the decarboxylation of oxaloacetate into malonic semi-aldehyde, or one of its salts, is:

(i) overexpressed and/or

(ii) under the control of a strong or inducible promoter;

said enzyme being of sequence SEQ ID NO:1

(SEQ ID NO: 1)
MASVHGTTYELLRRQGIDTVFGNPGSNELPFLKDFPEDFRYILALQEACVV
GIADGYAQASRKPAFINLHSAAGTGNAMGALSNAWNSHSPLIVTAGQQTRA
MIGVEAX₁X₂TNVDAANLPRPLVKWSYEPASAAEVPHAMSRAIHMASMAPQ
GPVYLSVPYDDWDKDADPQSHHLFDRHVSSSVRLNDQDLDILVKALNSASN
PAIVLGPDVDAANANADCVMLAERLKAPVWVAPSAPRCPFPTRHPCFRGLM
PAGIAAISQLLEGHDVVLVIGAPVFRYHQYDPGQYLKPGTRLISVTCDPLE
AARAPMGDAIVADIGAMASALANLVEESSRQLPTAAPEPAKVDQDAGRLHP
ETVFDTLNDMAPENAIYLNESX₃STTAQMWQRLNMRNPGSYYX₄X₅AAGG
X₆GFALPAAIGVQLAEPERQVIAVIGDGSANYSISALWTAAQYNIPTIFVI
MNNGTYGX₇LRWFAGVLEAENVPGLDVPGIDFRALAKGYGVQALKADNLEQ
LKGSLQEALSAKGPVLIEVSTVSPVK

wherein:
X₁ represents an amino acid selected from the group consisting of leucine, lysine, arginine and valine;
X₂ represents an amino acid selected from the group consisting of leucine and lysine;
X₃ represents an amino acid selected from the group consisting of threonine and serine;
X₄ represents an amino acid selected from the group consisting of phenylalanine, asparagine, alanine, isoleucine and valine;
X₅ represents an amino acid selected from the group consisting of cysteine and arginine;
X₆ represents an amino acid selected from the group consisting of leucine, asparagine and alanine; and
X₇ represents an amino acid selected from the group consisting of alanine and leucine, with the proviso that the enzyme cannot have the sequence SEQ ID NO: 1 wherein X₁ represents leucine; X₂ represents leucine; X₃ represents threonine; X₄ represents phenylalanine; X₅ represents cysteine; X₆ represents leucine; and X₇ represents alanine.

As illustrated in the enclosed examples, and thus the recombinant yeasts of the invention have the ability to produce malonic semi-aldehyde, one of its salts, or a malonic semi-aldehyde derivative.

Said advantageous property can be further increased by also recombining the yeast with additional modifications described here-after, in particular in order to obtain some malonic semi-aldehyde derivatives.

A recombinant yeast according to the invention can consequently advantageously be used in a method for producing malonic semi-aldehyde, one of its salts, or a malonic semi-aldehyde derivative as described here-after.

Accordingly, the invention further relates to a method for producing malonic semi-aldehyde, one of its salts, or a malonic semi-aldehyde derivative, said method comprising the steps of:

(a) culturing a recombinant yeast of the invention in a culture medium; and

(b) recovering the malonic semi-aldehyde, one of its salts, or a malonic semi-aldehyde derivative from said culture medium.

In a particular embodiment, the culture medium comprises at least a carbon source, preferably a carbon source selected from the group consisting of glucose and sucrose.

A further object of the present invention is the use of a recombinant yeast according to the invention for the production of malonic semi-aldehyde, one of its salts, or a malonic semi-aldehyde derivative.

As explained here-after, derivatives of malonic semi-aldehyde are compounds that can be obtained from malonic semi-aldehyde, or from one of its salts, after modification by at least one enzyme naturally and/or artificially present in the microorganism producing the malonic semi-aldehyde, or one of its salts, according to the invention, in particular in the yeast producing the malonic semi-aldehyde, or one of its salts, according to the invention. Accordingly, in the present invention, in order to obtain a derivative of malonic semi-aldehyde, it is first necessary to produce malonic semi-aldehyde and then, through at least one additional step, to transform malonic semi-aldehyde in one of its derivatives, in particular one of its derivatives mentioned in the present text.

Finally, a further object of the invention is an enzyme of the invention, characterized in that its sequence is SEQ ID NO: 105:

(SEQ ID NO: 105)
MASVHGTTYELLRRQGIDX₈VFGNPGSNELPFLKDFPEDFRYILALQEACV
VGIADGYAQASRKPAFINLHSAAGTGNAMGALSNAWNSHSPLIVTAGQQTR
AMIGVEAX₁X₂TNVDAANLPRPLVKWSYEPASAAEVPHAMSRAIHMASMAP
QGPVYLSVPYDDWDKDADPQSHHLFDRHVX₉SSVRLNDQDLDILVKALNSA
SNPX₁₀IVLGPDVDAANANADCVMLAERLKAPVWVAPSAPRCPFPTRHPCF
RGLMPAGIAAISQLLEGHDVVLVIGAPVFRYX₁₁X₁₂YDPGQYLKPGTRLI
SVTCDPLEAARAPMGDAIVADIGAMASALANLVEESSRQLPTAAPEPAKVD
QDAGRLHPETVFDTLNDMAPEX₁₃AIYLNESX₃STTAQMWQRLX₁₄MRNPG
SYYX₄X₅AAGGX₆GFALPAAIGVQLAEPX₁₅RQVIAVIGDGSANYSISALW
TAAQYNX₁₆PTIFVIMNNGTYGX₇LRWX₁₇AGVLX₁₈AENVPGLDVPGIDF
RALAKGYGVQALKADNLEQLKGSLQEALSAKGPVLIEVSTVSPVK

wherein:
X₁ represents an amino acid selected from the group consisting of leucine, lysine, arginine and valine;
X₂ represents an amino acid selected from the group consisting of leucine and lysine;
X₃ represents an amino acid selected from the group consisting of threonine and serine;
X₄ represents an amino acid selected from the group consisting of phenylalanine, asparagine, alanine, isoleucine, valine, leucine, tryptophan and arginine;
X₅ represents an amino acid selected from the group consisting of cysteine and arginine;
X₆ represents an amino acid selected from the group consisting of leucine, asparagine, alanine, valine and serine;
X₇ represents an amino acid selected from the group consisting of alanine, leucine, threonine, glycine and asparagine;
X₈ represents an amino acid selected from the group consisting of threonine and isoleucine;

X₉ represents an amino acid selected from the group consisting of serine and threonine;

X₁₀ represents an amino acid selected from the group consisting of alanine and valine;
X₁₁ represents an amino acid selected from the group consisting of histidine and arginine;
X₁₂ represents an amino acid selected from the group consisting of glutamine and arginine;
X₁₃ represents an amino acid selected from the group consisting of asparagine and aspartic acid;
X₁₄ represents an amino acid selected from the group consisting of asparagine and aspartic acid;
X₁₅ represents an amino acid selected from the group consisting of glutamic acid and glycine;
X₁₆ represents an amino acid selected from the group consisting of isoleucine and valine;
X₁₇ represents an amino acid selected from the group consisting of phenylalanine and serine; and
X₁₈ represents an amino acid selected from the group consisting of glutamic acid and glycine;
with the proviso that the enzyme cannot have the sequence SEQ ID NO: 105 wherein X₁ represents leucine; X₂ represents leucine; X₃ represents threonine; X₄ represents phenylalanine; X₅ represents cysteine; X₆ represents leucine; X₇ represents alanine, X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine and X₁₈ represents glutamic acid.

More particularly, the said enzyme of the invention is characterized in that its sequence is SEQ ID NO:1:

(SEQ ID NO: 1)
MASVHGTTYELLRRQGIDTVFGNPGSNELPFLKDFPEDFRYILALQEACVV
GIADGYAQASRKPAFINLHSAAGTGNAMGALSNAWNSHSPLIVTAGQQTRA
MIGVEAX₁X₂TNVDAANLPRPLVKWSYEPASAAEVPHAMSRAIHMASMAPQ
GPVYLSVPYDDWDKDADPQSHHLFDRHVSSSVRLNDQDLDILVKALNSASN
PAIVLGPDVDAANANADCVMLAERLKAPVWVAPSAPRCPFPTRHPCFRGLM
PAGIAAISQLLEGHDVVLVIGAPVFRYHQYDPGQYLKPGTRLISVTCDPLE
AARAPMGDAIVADIGAMASALANLVEESSRQLPTAAPEPAKVDQDAGRLHP
ETVFDTLNDMAPENAIYLNESX₃STTAQMWQRLNMRNPGSYYX₄X₅AAGG
X₆GFALPAAIGVQLAEPERQVIAVIGDGSANYSISALWTAAQYNIPTIFVI
MNNGTYGX₇LRWFAGVLEAENVPGLDVPGIDFRALAKGYGVQALKADNLEQ
LKGSLQEALSAKGPVLIEVSTVSPVK

wherein:
X₁ represents an amino acid selected from the group consisting of leucine, lysine, arginine and valine;
X₂ represents an amino acid selected from the group consisting of leucine and lysine;
X₃ represents an amino acid selected from the group consisting of threonine and serine;
X₄ represents an amino acid selected from the group consisting of phenylalanine, asparagine, alanine, isoleucine and valine;
X₅ represents an amino acid selected from the group consisting of cysteine and arginine;
X₆ represents an amino acid selected from the group consisting of leucine, asparagine and alanine; and
X₇ represents an amino acid selected from the group consisting of alanine and leucine,

with the proviso that the enzyme cannot have the sequence SEQ ID NO: 1 wherein X₁ represents leucine; X₂ represents leucine; X₃ represents threonine; X₄ represents phenylalanine; X₅ represents cysteine; X₆ represents leucine; and X₇ represents alanine.

### CONTENT OF THE FIGURES

FIG. 1 illustrates the steps allowing the transformation of malonic semi-aldehyde or one of its salts into 3-hydroxypropionate, 3-hydroxypropionate, Acrylyl-CoA, Propyonyl-CoA, propanal and propanol in a yeast according to the invention.

FIG. 2 illustrates the steps allowing the transformation of malonic ami-aldehyde or one of its salts into acrylic acid and acrylate in a yeast according to the invention.

FIG. 3 illustrate the catalytic results obtained with various enzymes according to the invention in separate yeast extracts of the invention. For each tested enzyme, the Vi saturation curve is illustrated as a function of the concentration of the substrate (oxaloacetate—2.5, 5, 10, 20, 40, 80 and 120 mM). The enzymes tested are Enzyme No 1 (FIG. 3a), Enzyme No 6 (FIG. 3b), Enzyme No 7 (FIG. 3c), Enzyme No 8 (FIG. 3d) and Enzyme No 9 (FIG. 3e).

Ordinate: Vi (nmole·min⁻¹·mg⁻¹)

Abscissa: oxaloacetate (mM)

### DETAILED DESCRIPTION OF THE INVENTION

The inventors have conceived artificial enzymes, and genetically modified microorganisms, and especially genetically modified yeasts, comprising them.

These enzymes provide the microorganisms, and in particular provide the yeasts, with the capacity, or an increase capacity as compared to the parent yeasts, to catalyze the transformation of oxaloacetate into malonic semi-aldehyde, or one of its salts. The recombinant yeasts of the invention are thus able to produce malonic semi-aldehyde, one of its salts, or a malonic semi-aldehyde derivative.

These enzymes and these genetically modified microorganisms, including these genetically modified yeasts, are described throughout the present specification.

### Definitions

The term “microorganism”, as used herein, refers to a yeast which is not modified artificially. The microorganism may be “donor” if it provides genetic element to be integrated in the microorganism “acceptor” which will express this foreign genetic element or if it used as tool for genetic constructions or protein expressions. The microorganism of the invention is chosen among yeast which expresses genes for the biosynthesis of oxaloacetate.

The term “recombinant microorganism” or “genetically modified microorganism” or “recombinant yeast” or “genetically modified yeast”, as used herein, refers to a yeast genetically modified or genetically engineered. It means, according to the usual meaning of these terms, that the microorganism of the invention is not found in nature and is modified either by introduction or by deletion or by modification of genetic elements from equivalent microorganism found in nature or from artificial sequences, and in particular by the introduction of genetic elements either artificial or originating from other microorganisms.

A microorganism may be modified to express exogenous genes if these genes are introduced into the microorganism with all the elements allowing their expression in the host microorganism. A microorganism may be modified to modulate the expression level of an endogenous gene. The modification or “transformation” of microorganism, like yeast, with exogenous DNA is a routine task for those skilled in the art. In particular, a genetic modification of a microorganism according to the invention, more particularly the genetic modification(s) herein defined, may be carried out by using CRISPR-Cas systems, as described in DiCarlo et al. (Nucl. Acids Res., vol. 41, No. 7, 2013: 4336-4343).

The term “endogenous gene” means that the gene was present in the microorganism before any genetic modification, in the wild-type strain.

The term “exogenous gene” means that the gene was introduced into a microorganism, by means well known by the man skilled in the art, whereas this gene is not naturally occurring in the wild-type microorganism. Microorganism can express exogenous genes if these genes are introduced into the microorganism with all the elements allowing their expression in the host microorganism. As mentioned above, transforming microorganisms with exogenous DNA is a routine task for the man skilled in the art. Exogenous genes may be integrated into the host chromosome, or be expressed extra-chromosomally from plasmids or vectors. A variety of plasmids, which differ with respect to their origin of replication and their copy number in the cell, are all known in the art. The sequence of exogenous genes may be adapted for its expression in the host microorganism. Indeed, the man skilled in the art knows the notion of codon usage bias and how to adapt nucleic sequences for a particular codon usage bias without modifying the deduced protein.

The term “heterologous gene” means that the gene is derived from a species of microorganism different from the recipient microorganism that expresses it. It refers to a gene which is not naturally occurring in the microorganism.

In the present application, the genes may be referenced with their common names and with references to their nucleotide sequences and, the case arising, to their amino acid sequences. Using the references given in accession number for known genes, those skilled in the art are able to determine the equivalent genes in other organisms, bacterial strains, yeast, fungi, mammals, plants, etc. This routine work is advantageously done using consensus sequences that can be determined by carrying out sequence alignments with genes derived from other microorganisms and designing degenerated probes to clone the corresponding gene in another organism.

The man skilled in the art knows different means to modulate, and in particular up-regulate or down-regulate, the expression of endogenous or exogenous genes. For example, a way to enhance expression of endogenous or exogenous genes is to introduce one or more supplementary copies of the gene onto a chromosome or a plasmid.

Another way is to replace the endogenous promoter of a gene with, or to use as exogeneous promoter of a gene, a stronger promoter. These promoters may be homologous or heterologous. Promoters particularly interesting in the present invention are described in more detail elsewhere in the present specification.

The nucleic acid expression construct may further comprise 5′ and/or 3′ recognition sequences and/or selection markers.

The term “overexpression” means that the expression of a gene or of an enzyme is increased as compared to the non-modified microorganism. Increasing the expression of an enzyme is obtained by increasing the expression of a gene encoding said enzyme. When a non-modified microorganism did not expressed a given gene, modifying said microorganism in order for it to express this gene is thus also considered as being an increase expression of said gene, and thus as an overexpression. Increasing the expression of a gene may be carried out by all techniques known by the one skilled in the art. In this regard, it may be notably cited the implementation of a strong promoter upstream the nucleic acid intended to be overexpressed or the introduction of a plurality of copies of the said nucleic acid between a promoter, especially a strong promoter, and a terminator. The term “inducible promoter” is used to qualify a promoter whose activity is induced, i.e. increased:
in the presence of one or more particular metabolite(s). The higher the metabolite concentration in the medium, the stronger the promoter activity; or
in the presence of a low concentration, or in the absence, of one or more metabolite(s). These metabolites are different from those whose increasing presence induces the activity of the promoter. The lower the metabolite concentration in the medium, the stronger the promoter activity.

The “activity” of an enzyme is used interchangeably with the term “function” and designates, in the context of the invention, the capacity of an enzyme to catalyze a desired reaction.

The term “enhanced activity” of an enzyme designates either an increased specific catalytic activity of the enzyme, and/or an increased quantity/availability of the enzyme in the cell, obtained for example by overexpression of the gene encoding the enzyme.

The terms “encoding” or “coding” refer to the process by which a polynucleotide, through the mechanisms of transcription and translation, produces an amino-acid sequence.

The gene(s) encoding the enzyme(s) considered in the present invention can be exogenous or endogenous. An exogenous gene can be artificial.

An artificial gene is well known to the man skilled in the art and designate a gene obtained through create a gene in a laboratory.

In some embodiments, the microorganism in which the above-mentioned DNA construct(s) is/are intended to be inserted may advantageously comprises one or more selectable markers useful for the selection of transformed microbial cells. Preferably, said selectable marker(s) are comprised in the DNA construct(s) according to the present invention.

In some embodiments, the selectable marker is an antibiotic resistance marker. Illustrative examples of antibiotic resistance markers include, but are not limited to the, NAT1, AUR1-C, HPH, DSDA, KAN<R>, and SH BLE gene products. The NAT 1 gene product from S. noursei confers resistance to nourseothricin; the AURl-C gene product from Saccharomyces cerevisiae confers resistance to Auerobasidin A (AbA); the HPH gene product of Klebsiella pneumonia confers resistance to Hygromycin B; the DSDA gene product of E. coli allows cells to grow on plates with D-serine as the sole nitrogen source; the KAN<R> gene of the Tn903 transposon confers resistance to G418; and the SH BLE gene product from Streptoalloteichus hindustanus confers resistance to Zeocin (bleomycin).

In some embodiments, the antibiotic resistance marker is deleted after the genetically modified microbial cell of the invention is isolated. The man skilled in the art is able to choose suitable marker in specific genetic context.

In some embodiments, the selectable marker rescues an auxotrophy (e.g., a nutritional auxotrophy) in the genetically modified microbial cell. In such embodiments, a parent microbial cell comprises a functional disruption in one or more gene products that function in an amino acid or nucleotide biosynthetic pathway, such as, for example, the HIS3, LEU2, LYS1, LYS2, MET 15, TRP1, ADE2, and URA3 gene products in yeast, which renders the parent microbial cell incapable of growing in media without supplementation with one or more nutrients (auxotrophic phenotype). The auxotrophic phenotype can then be rescued by transforming the parent microbial cell with a chromosomal integration encoding a functional copy of the disrupted gene product (NB: the functional copy of the gene can originate from close species, such as Kluveromyces, Candida etc.), and the genetically modified microbial cell generated can be selected for based on the loss of the auxotrophic phenotype of the parent microbial cell.

For each of the nucleic acid sequences comprising a promoter sequence, a coding sequence (e.g. an enzyme coding sequence), or a terminator sequence, reference sequences are described herein.

For each or the amino acid sequences of interest, reference sequences are described herein.

For obvious reasons, in all the present description, a specific nucleic acid sequence or a specific amino acid sequence which complies with, respectively, the considered nucleotide or amino acid identity, should further lead to obtaining a protein (or enzyme) which displays the desired biological activity.

The “fermentation” or “culture” is generally conducted in fermenters with an appropriate culture medium adapted to the microorganism being cultivated, containing at least one simple carbon source, and if necessary co-substrates.

Microorganisms disclosed herein may be grown in fermentation media for the production of a product from oxaloacetate. Fermentation media, or “culture medium”, for the present cells may contain at least about 10 g/L of glucose. Additional carbon substrates may include but are not limited to monosaccharides such as fructose, mannose, xylose and arabinose; oligosaccharides such as lactose maltose, galactose, or sucrose; polysaccharides such as starch or cellulose or mixtures thereof and unpurified mixtures from renewable feedstocks such as cheese whey permeate cornsteep liquor, sugar beet molasses, and barley malt.

Hence, it is contemplated that the source of carbon utilized in the present invention may encompass a wide variety of carbon containing substrates and will only be limited by the choice of organism.

Although it is contemplated that all of the above-mentioned carbon substrates and mixtures thereof are suitable in the present invention, preferred carbon substrates are glucose, fructose, and sucrose, or mixtures of these with C5 sugars such as xylose and/or arabinose for microorganisms modified to use C5 sugars, and more particularly glucose.

A preferred carbon substrate is glucose.

In addition to an appropriate carbon source, fermentation media may contain suitable minerals, salts, cofactors, buffers and other components, known to those skilled in the art, suitable for the growth of the cultures and promotion of the enzymatic pathway necessary for the production of the desired product.

Besides, additional genetic modifications suitable for the growth of recombinant microorganisms according to the invention may be considered.

The terms “Aerobic conditions” refers to concentrations of oxygen in the culture medium that are sufficient for an aerobic or facultative anaerobic microorganism to use di-oxygene as a terminal electron acceptor.

“Microaerobic condition” refers to a culture medium in which the concentration of oxygen is less than that in air, i.e. oxygen concentration up to 6% 02.

An “appropriate culture medium” designates a medium (e.g. a sterile, liquid medium) comprising nutrients essential or beneficial to the maintenance and/or growth of the cell such as carbon sources or carbon substrate, nitrogen sources, for example, peptone, yeast extracts, meat extracts, malt extracts, urea, ammonium sulfate, ammonium chloride, ammonium nitrate and ammonium phosphate; phosphorus sources, for example, monopotassium phosphate or dipotassium phosphate; trace elements (e.g., metal salts), for example magnesium salts, cobalt salts and/or manganese salts; as well as growth factors such as amino acids, vitamins, growth promoters, and the like. The term “carbon source” or “carbon substrate” or “source of carbon” according to the present invention denotes any source of carbon that can be used by those skilled in the art to support the normal growth of a microorganism, including hexoses (such as glucose, galactose or lactose), pentoses, monosaccharides, oligosaccharides, disaccharides (such as sucrose, cellobiose or maltose), molasses, starch or its derivatives, cellulose, hemicelluloses and combinations thereof.

### General Features of Genetic Modifications Introduced According to the Invention

The at least one nucleic acid encoding an enzyme able to catalyze the decarboxylation of oxaloacetate into malonic semi-aldehyde, or one of its salts, can be expressed in a yeast using two kinds of non mutually exclusive manners:
It is overexpressed, i.e. one or a plurality of copy(ies) of is/are introduced in the said microorganism; and/or
The said at least one copy is under the control of a strong or inducible promoter.

When the yeast is for example Saccharomyces cerevisiae, genes (i.e. nucleic acid sequences) introduced in the yeast genome and originating from other organisms than Saccharomyces cerevisiae are generally “transcoded” (generally codon-optimized”), meaning the these genes are synthesized with an optimal codon usage for expression S. cerevisiae. The nucleotide sequence (and not the protein sequence) of some genes from S. cerevisiae has also been modified (“transcoded”) to minimize recombination with an endogenous copy of the said gene.

A gene may be rendered “inducible” by deleting the endogenous copy of the gene (if necessary) and placing a new copy of the ORF under the control of an inducible promoter.

### Enzymes According to the Invention

The inventors have conceived novel enzymes able to catalyze the decarboxylation of oxaloacetate into malonic semi-aldehyde in a yeast.

This family of enzymes is characterized in that they are of sequence SEQ ID NO: 105:

(SEQ ID NO: 105)
MASVHGTTYELLRRQGIDX₈VFGNPGSNELPFLKDFPEDFRYILALQEACV
VGIADGYAQASRKPAFINLHSAAGTGNAMGALSNAWNSHSPLIVTAGQQTR
AMIGVEAX₁X₂TNVDAANLPRPLVKWSYEPASAAEVPHAMSRAIHMASMAP
QGPVYLSVPYDDWDKDADPQSHHLFDRHVX₉SSVRLNDQDLDILVKALNSA
SNPX₁₀IVLGPDVDAANANADCVMLAERLKAPVWVAPSAPRCPFPTRHPCF
RGLMPAGIAAISQLLEGHDVVLVIGAPVFRYX₁₁X₁₂YDPGQYLKPGTRLI
SVTCDPLEAARAPMGDAIVADIGAMASALANLVEESSRQLPTAAPEPAKVD
QDAGRLHPETVFDTLNDMAPEX₁₃AIYLNESX₃STTAQMWQRLX₁₄MRNPG
SYYX₄X₅AAGGX₆GFALPAAIGVQLAEPX₁₅RQVIAVIGDGSANYSISALW
TAAQYNX₁₆PTIFVIMNNGTYGX₇LRWX₁₇AGVLX₁₈AENVPGLDVPGIDF
RALAKGYGVQALKADNLEQLKGSLQEALSAKGPVLIEVSTVSPVK

wherein:
X₁ represents an amino acid selected from the group consisting of leucine, lysine, arginine and valine;
X₂ represents an amino acid selected from the group consisting of leucine and lysine;
X₃ represents an amino acid selected from the group consisting of threonine and serine;
X₄ represents an amino acid selected from the group consisting of phenylalanine, asparagine, alanine, isoleucine, valine, leucine, tryptophan and arginine;
X₅ represents an amino acid selected from the group consisting of cysteine and arginine;
X₆ represents an amino acid selected from the group consisting of leucine, asparagine, alanine, valine and serine;
X₇ represents an amino acid selected from the group consisting of alanine, leucine, threonine, glycine and asparagine;
X₈ represents an amino acid selected from the group consisting of threonine and isoleucine;
X₉ represents an amino acid selected from the group consisting of serine and threonine;
X₁₀ represents an amino acid selected from the group consisting of alanine and valine;
X₁₁ represents an amino acid selected from the group consisting of histidine and arginine;
X₁₂ represents an amino acid selected from the group consisting of glutamine and arginine;
X₁₃ represents an amino acid selected from the group consisting of asparagine and aspartic acid;
X₁₄ represents an amino acid selected from the group consisting of asparagine and aspartic acid;
X₁₅ represents an amino acid selected from the group consisting of glutamic acid and glycine;
X₁₆ represents an amino acid selected from the group consisting of isoleucine and valine;
X₁₇ represents an amino acid selected from the group consisting of phenylalanine and serine; and
X₁₈ represents an amino acid selected from the group consisting of glutamic acid and glycine;

with the proviso that the enzyme cannot have the sequence SEQ ID NO: 105 wherein X₁ represents leucine; X₂ represents leucine; X₃ represents threonine; X₄ represents phenylalanine; X₅ represents cysteine; X₆ represents leucine; X₇ represents alanine, X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine and X₁₈ represents glutamic acid.

In an embodiment of the invention, an enzyme of sequence of SEQ ID NO: 105 is such that X₉ represents serine.

In an embodiment of the invention, an enzyme of sequence of SEQ ID NO: 105 is such that X₁₀ represents alanine.

In a particular embodiment, an enzyme of sequence SEQ ID NO: 105 as defined above is such that X₉ represents serine and X₁₀ represents alanine.

In an embodiment of the invention, an enzyme of sequence of SEQ ID NO: 105 is such that X₁ represents arginine.

In a particular embodiment, an enzyme of sequence SEQ ID NO: 105 as defined above is such that X₁ represents arginine, X₉ represents serine and X₁₀ represents alanine.

In an embodiment of the invention, an enzyme of sequence of SEQ ID NO: 105 is such that X₆ represents leucine or asparagine, and in particular represents leucine.

In a particular embodiment, an enzyme of sequence SEQ ID NO: 105 as defined above is such that X₁ represents arginine, X₆ represents leucine, X₉ represents serine and X₁₀ represents alanine.

In a particular embodiment, an enzyme of sequence SEQ ID NO: 105 as defined above is such that X₇ represents alanine, leucine, threonine or glycine, in particular represents alanine.

In an embodiment of the invention, an enzyme of sequence SEQ ID NO: 105 as defined above is such that when X₈ represents isoleucine, X₁₄ represents aspartic acid.

In a particular embodiment, an enzyme of sequence SEQ ID NO: 105 is such that X₄ represents phenylalanine, asparagine, leucine, alanine, tryptophan or arginine, in particular phenylalanine, asparagine or leucine, and more particularly phenylalanine or leucine.

In a particular embodiment, an enzyme of sequence SEQ ID NO: 105 is such that X₂ represents leucine.

In a particular embodiment, an enzyme of sequence SEQ ID NO: 105 is such that X₃ represents threonine.

In a particular embodiment, an enzyme of sequence SEQ ID NO: 105 is such that X₅ represents cysteine.

In a particular embodiment, an enzyme of sequence SEQ ID NO: 105 is such that X₂ represents leucine, X₃ represents threonine and X₅ represents cysteine.

In a particular embodiment, an enzyme of sequence SEQ ID NO: 105 as defined above is such that X₁ represents arginine, X₂ represents leucine, X₃ represents threonine, X₅ represents cysteine, X₆ represents leucine, X₉ represents serine and X₁₀ represents alanine.

In another embodiment, an enzyme of sequence SEQ ID NO: 105 as defined above is such that:
X₁ represents arginine,
X₂ represents leucine,
X₃ represents threonine,
X₄ represents phenylalanine, asparagine, leucine, alanine, tryptophan or arginine, in particular phenylalanine, asparagine or leucine, and more particularly phenylalanine or leucine,
X₅ represents cysteine,
X₆ represents leucine,
X₉ represents serine; and
X₁₀ represents alanine.

In another embodiment, an enzyme of sequence SEQ ID NO: 105 as defined above is such that:
X₁ represents arginine,
X₂ represents leucine,
X₃ represents threonine,
X₄ represents phenylalanine, asparagine, leucine, alanine, tryptophan or arginine, in particular phenylalanine, asparagine or leucine, and more particularly phenylalanine or leucine,
X₅ represents cysteine,
X₆ represents leucine,
X₇ represents alanine, leucine, threonine or glycine,
X₉ represents serine; and
X₁₀ represents alanine.

An enzyme according to the invention can in particular be selected from the group consisting of:

(i) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents alanine; X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 2);

(ii) an enzyme of sequence SEQ ID NO: 105 wherein X₁ represents arginine; X₂ represents leucine; X₃ represents threonine; X₄ represents leucine; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glutamic acid; X₁₆ represents valine; X₁₇ represents phenylalanine; and X₁₈ represents glutamic acid (i.e. an enzyme of amino acid sequence SEQ ID NO: 106);

(iii) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents leucine; X₈ represents isoleucine; X₉ represents serine; X₁₀ represents alanine; X₁₁ represents arginine; X₁₂ represents glutamine; X₁₃ represents asparagine; X₁₄ represents aspartic acid; X₁₅ represents glycine; X₁₆ represents isoleucine; X₁₇ represents serine; and X₁₈ represents glycine (i.e. an enzyme of amino acid sequence SEQ ID NO: 107);

(iv) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents glycine; X₈ represents threonine; X₉ represents serine; X₁₀ represents alanine; X₁₁ represents arginine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 108);

(v) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 109);

(vi) an enzyme of sequence SEQ ID NO: 1 wherein X₁ represents arginine; X₂ represents leucine; X₃ represents threonine; X₄ represents asparagine; X₅ represents cysteine; X₆ represents leucine; X₇ represents alanine; X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 3);

(vii) an enzyme of sequence SEQ ID NO: 1 wherein X₁ represents arginine; X₂ represents leucine; X₃ represents threonine; X₄ represents asparagine; X₅ represents cysteine; X₆ represents leucine; X₇ represents alanine; X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 4);

(viii) an enzyme of sequence SEQ ID NO: 1 wherein X₁ represents arginine; X₂ represents leucine; X₃ represents threonine; X₄ represents asparagine; X₅ represents cysteine; X₆ represents leucine; X₇ represents leucine; X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 5);

(ix) an enzyme of sequence SEQ ID NO: 1 wherein X₁ represents valine; X₂ represents lysine; X₃ represents threonine; X₄ represents asparagine; X₅ represents cysteine; X₆ represents asparagine; X₇ represents alanine; X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 6).

(x) an enzyme of sequence SEQ ID NO: 1 wherein X₁ represents arginine; X₂ represents leucine; X₃ represents threonine; X₄ represents alanine; X₅ represents cysteine; X₆ represents leucine; X₇ represents alanine; X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 110);

(xi) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents leucine; X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 111);

(xii) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents alanine; X₈ represents threonine; X₉ represents serine; X₁₀ represents alanine; X₁₁ represents arginine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 112);

(xiii) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents threonine; X₈ represents threonine; X₉ represents serine; X₁₀ represents alanine; X₁₁ represents arginine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 113);

(xiv) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents alanine; X₈ represents threonine; X₉ represents serine; X₁₀ represents alanine; X₁₁ represents histidine; X₁₂ represents arginine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 114);

(xv) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents leucine; X₈ represents threonine; X₉ represents serine; X₁₀ represents alanine; X₁₁ represents histidine; X₁₂ represents arginine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 115);

(xvi) an enzyme of sequence SEQ ID NO: 105 wherein X₁ represents arginine; X₂ represents leucine; X₃ represents threonine; X₄ represents leucine; X₅ represents cysteine; X₆ represents leucine; X₇ represents leucine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glutamic acid; X₁₆ represents valine; X₁₇ represents phenylalanine; and X₁₈ represents glutamic acid (i.e. an enzyme of amino acid sequence SEQ ID NO: 116);

(xvii) an enzyme of sequence SEQ ID NO: 105 wherein X₁ represents arginine; X₂ represents leucine; X₃ represents threonine; X₄ represents leucine; X₅ represents cysteine; X₆ represents leucine; X₇ represents glycine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glutamic acid; X₁₆ represents valine; X₁₇ represents phenylalanine; and X₁₈ represents glutamic acid (i.e. an enzyme of amino acid sequence SEQ ID NO: 117);

(xviii) an enzyme of sequence SEQ ID NO: 105 wherein X₁ represents arginine; X₂ represents leucine; X₃ represents threonine; X₄ represents leucine; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glutamic acid; X₁₆ represents valine; X₁₇ represents serine; and X₁₈ represents glutamic acid (i.e. an enzyme of amino acid sequence SEQ ID NO: 118);

(xix) an enzyme of sequence SEQ ID NO: 105 wherein X₁ represents arginine; X₂ represents leucine; X₃ represents threonine; X₄ represents leucine; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glycine; X₁₆ represents valine; X₁₇ represents serine; and X₁₈ represents glycine (i.e. an enzyme of amino acid sequence SEQ ID NO: 119);

(xx) an enzyme of sequence SEQ ID NO: 105 wherein X₁ represents arginine; X₂ represents leucine; X₃ represents threonine; X₄ represents leucine; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glycine; X₁₆ represents isoleucine; X₁₇ represents serine; and X₁₈ represents glycine (i.e. an enzyme of amino acid sequence SEQ ID NO: 120);

(xxi) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents leucine; X₈ represents isoleucine; X₉ represents serine; X₁₀ represents alanine; X₁₁ represents histidine; X₁₂ represents arginine; X₁₃ represents asparagine; X₁₄ represents aspartic acid; X₁₅ represents glycine; X₁₆ represents isoleucine; X₁₇ represents serine; and X₁₈ represents glycine (i.e. an enzyme of amino acid sequence SEQ ID NO: 121);

(xxii) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents leucine; X₈ represents isoleucine; X₉ represents serine; X₁₀ represents alanine; X₁₁ represents histidine; X₁₂ represents arginine; X₁₃ represents asparagine; X₁₄ represents aspartic acid; X₁₅ represents glycine; X₁₆ represents isoleucine; X₁₇ represents serine; and X₁₈ represents glycine (i.e. an enzyme of amino acid sequence SEQ ID NO: 122);

(xxiii) an enzyme of sequence SEQ ID NO: 105 wherein X₁ represents arginine; X₂ represents leucine; X₃ represents threonine; X₄ represents tryptophan; X₅ represents cysteine; X₆ represents leucine; X₇ represents alanine; X₈ represents threonine; X₉ represents serine; X₁₀ represents alanine; X₁₁ represents arginine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 123);

(xxiv) an enzyme of sequence SEQ ID NO: 105 wherein X₁ represents arginine; X₂ represents leucine; X₃ represents threonine; X₄ represents arginine; X₅ represents cysteine; X₆ represents valine; X₇ represents alanine; X₈ represents threonine; X₉ represents serine; X₁₀ represents alanine; X₁₁ represents arginine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 124);

(xxv) an enzyme of sequence SEQ ID NO: 105 wherein X₁ represents arginine; X₂ represents leucine; X₃ represents threonine; X₄ represents tryptophan; X₅ represents cysteine; X₆ represents leucine; X₇ represents asparagine; X₈ represents isoleucine; X₉ represents serine; X₁₀ represents alanine; X₁₁ represents histidine; X₁₂ represents arginine; X₁₃ represents asparagine; X₁₄ represents aspartic acid; X₁₅ represents glycine; X₁₆ represents isoleucine; X₁₇ represents serine; and X₁₈ represents glycine (i.e. an enzyme of amino acid sequence SEQ ID NO: 125);

(xxvi) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents threonine; X₈ represents threonine; X₉ represents threonine; X₁₀ represents valine; X₁₁ represents histidine; X₁₂ represents arginine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 126);

(xxvii) an enzyme of sequence SEQ ID NO: 105 wherein X₁ represents arginine; X₂ represents leucine; X₃ represents threonine; X₄ represents alanine; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 127);

(xxviii) an enzyme of sequence SEQ ID NO: 105 wherein X₁ represents arginine; X₂ represents leucine; X₃ represents threonine; X₄ represents leucine; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 128);

(xxix) an enzyme of sequence SEQ ID NO: 105 wherein X₁ represents arginine; X₂ represents leucine; X₃ represents threonine; X₄ represents arginine; X₅ represents cysteine; X₆ represents valine; X₇ represents alanine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glutamic acid; X₁₆ represents valine; X₁₇ represents phenylalanine; and X₁₈ represents glutamic acid (i.e. an enzyme of amino acid sequence SEQ ID NO: 129);

(xxx) an enzyme of sequence SEQ ID NO: 105 wherein X₁ represents arginine; X₂ represents leucine; X₃ represents threonine; X₄ represents arginine; X₅ represents cysteine; X₆ represents valine; X₇ represents glycine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glutamic acid; X₁₆ represents valine; X₁₇ represents phenylalanine; and X₁₈ represents glutamic acid (i.e. an enzyme of amino acid sequence SEQ ID NO: 130);

(xxxi) an enzyme of sequence SEQ ID NO: 105 wherein X₁ represents arginine; X₂ represents leucine; X₃ represents threonine; X₄ represents tryptophan; X₅ represents cysteine; X₆ represents serine; X₇ represents asparagine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glutamic acid; X₁₆ represents valine; X₁₇ represents phenylalanine; and X₁₈ represents glutamic acid (i.e. an enzyme of amino acid sequence SEQ ID NO: 131);

(xxxii) an enzyme of sequence SEQ ID NO: 105 wherein X₁ represents arginine; X₂ represents leucine; X₃ represents threonine; X₄ represents tryptophan; X₅ represents cysteine; X₆ represents leucine; X₇ represents glycine; X₈ represents threonine; X₉ represents serine; X₁₀ represents alanine; X₁₁ represents arginine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 132);

(xxxiii) an enzyme of sequence SEQ ID NO: 105 wherein X₁ represents arginine; X₂ represents leucine; X₃ represents threonine; X₄ represents arginine; X₅ represents cysteine; X₆ represents leucine; X₇ represents glycine; X₈ represents threonine; X₉ represents serine; X₁₀ represents alanine; X₁₁ represents arginine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 133);

(xxxiv) an enzyme of sequence SEQ ID NO: 105 wherein X₁ represents arginine; X₂ represents leucine; X₃ represents threonine; X₄ represents tryptophan; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 134);

(xxxv) an enzyme of sequence SEQ ID NO: 105 wherein X₁ represents arginine; X₂ represents leucine; X₃ represents threonine; X₄ represents arginine; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents asparagine; X₁₄ represents asparagine; X₁₅ represents glutamic acid; X₁₆ represents isoleucine; X₁₇ represents phenylalanine; and X₁₈ represents glutamic acid (i.e. an enzyme of amino acid sequence SEQ ID NO: 135);

(xxxvi) an enzyme of sequence SEQ ID NO: 105 wherein X₁ represents arginine; X₂ represents leucine; X₃ represents threonine; X₄ represents arginine; X₅ represents cysteine; X₆ represents valine; X₇ represents threonine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glutamic acid; X₁₆ represents valine; X₁₇ represents phenylalanine; and X₁₈ represents glutamic acid (i.e. an enzyme of amino acid sequence SEQ ID NO: 136);

and

(xxxvii) an enzyme of sequence SEQ ID NO: 105 wherein X₁ represents arginine; X₂ represents leucine; X₃ represents threonine; X₄ represents tryptophan; X₅ represents cysteine; X₆ represents serine; X₇ represents threonine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glutamic acid; X₁₆ represents valine; X₁₇ represents phenylalanine; and X₁₈ represents glutamic acid (i.e. an enzyme of amino acid sequence SEQ ID NO: 137).

An enzyme according to the invention can more particularly be selected from the group consisting of:

(i) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents alanine; X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid; (i.e. an enzyme of amino acid sequence SEQ ID NO: 2);

(ii) an enzyme of sequence SEQ ID NO: 105 wherein X₁ represents arginine; X₂ represents leucine; X₃ represents threonine; X₄ represents leucine; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents aspartic acid; X₁₄ represents asparagine; X₁₅ represents glutamic acid; X₁₆ represents valine; X₁₇ represents phenylalanine; and X₁₈ represents glutamic acid; (i.e. an enzyme of amino acid sequence SEQ ID NO: 106);

(iii) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents leucine; X₈ represents isoleucine; X₉ represents serine; X₁₀ represents alanine; X₁₁ represents arginine; X₁₂ represents glutamine; X₁₃ represents asparagine; X₁₄ represents aspartic acid; X₁₅ represents glycine; X₁₆ represents isoleucine; X₁₇ represents serine; and X₁₈ represents glycine; (i.e. an enzyme of amino acid sequence SEQ ID NO: 107);

(iv) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents glycine; X₈ represents threonine; X₉ represents serine; X₁₀ represents alanine; X₁₁ represents arginine; X₁₂ represents glutamine; X₁₃ represents asparagine; X₁₄ represents asparagine; X₁₅ represents glutamic acid; X₁₆ represents isoleucine; X₁₇ represents phenylalanine; and X₁₈ represents glutamic acid; (i.e. an enzyme of amino acid sequence SEQ ID NO: 108); and

(v) an enzyme of sequence SEQ ID NO: 105 wherein X₁ represents arginine; X₂ represents leucine; X₃ represents threonine; X₄ represents phenylalanine; X₅ represents cysteine; X₆ represents leucine; X₇ represents threonine; X₈ represents threonine; X₉ represents serine; X₁₀ represents alanine; X₁₁ represents histidine; X₁₂ represents arginine; X₁₃ represents asparagine; X₁₄ represents asparagine; X₁₅ represents glutamic acid; X₁₆ represents isoleucine; X₁₇ represents phenylalanine; and X₁₈ represents glutamic acid; (i.e. an enzyme of amino acid sequence SEQ ID NO: 109).

In a particular embodiment, the enzymes according to the invention are characterized in that they are of sequence SEQ ID NO: 1:

(SEQ ID NO: 1)
MASVHGTTYELLRRQGIDTVFGNPGSNELPFLKDFPEDFRYILALQEACVV
GIADGYAQASRKPAFINLHSAAGTGNAMGALSNAWNSHSPLIVTAGQQTRA
MIGVEAX₁X₂TNVDAANLPRPLVKWSYEPASAAEVPHAMSRAIHMASMAPQ
GPVYLSVPYDDWDKDADPQSHHLFDRHVSSSVRLNDQDLDILVKALNSASN
PAIVLGPDVDAANANADCVMLAERLKAPVWVAPSAPRCPFPTRHPCFRGLM
PAGIAAISQLLEGHDVVLVIGAPVFRYHQYDPGQYLKPGTRLISVTCDPLE
AARAPMGDAIVADIGAMASALANLVEESSRQLPTAAPEPAKVDQDAGRLHP
ETVFDTLNDMAPENAIYLNESX₃STTAQMWQRLNMRNPGSYYX₄X₅AAGG
X₆GFALPAAIGVQLAEPERQVIAVIGDGSANYSISALWTAAQYNIPTIFVI
MNNGTYGX₇LRWFAGVLEAENVPGLDVPGIDFRALAKGYGVQALKADNLEQ
LKGSLQEALSAKGPVLIEVSTVSPVK

wherein:
X₁ represents an amino acid selected from the group consisting of leucine, lysine, arginine and valine;
X₂ represents an amino acid selected from the group consisting of leucine and lysine;
X₃ represents an amino acid selected from the group consisting of threonine and serine;
X₄ represents an amino acid selected from the group consisting of phenylalanine, asparagine, alanine, isoleucine and valine;
X₅ represents an amino acid selected from the group consisting of cysteine and arginine;
X₆ represents an amino acid selected from the group consisting of leucine, asparagine and alanine; and
X₇ represents an amino acid selected from the group consisting of alanine and leucine,

with the proviso that the enzyme cannot have the sequence SEQ ID NO: 1 wherein X₁ represents leucine; X₂ represents leucine; X₃ represents threonine; X₄ represents phenylalanine; X₅ represents cysteine; X₆ represents leucine; and X₇ represents alanine.

According to an embodiment, X₁ in sequence SEQ ID NO: 1 represents an amino acid selected from the group consisting of lysine, arginine and valine. In particular, X₁ in sequence SEQ ID NO: 1 represents an amino acid selected from the group consisting of arginine and valine. Preferably, X₁ in sequence SEQ ID NO: 1 is arginine.

In a particular embodiment, an enzyme of the invention is of SEQ ID NO: 1 as defined above, with X₁ being arginine.

In a particular embodiment, X₂ in sequence SEQ ID NO: 1 represents leucine.

In another embodiment, in sequence SEQ ID NO: 1, X₁ represents valine and X₂ represents lysine.

In a particular embodiment, X₃ in sequence SEQ ID NO: 1 represents threonine.

In a particular embodiment, in sequence SEQ ID NO: 1, X₁ is arginine, X₂ represents leucine and X₃ represents threonine.

In an embodiment of the invention, X₄ in sequence SEQ ID NO: 1 represents phenylalanine or asparagine.

In an embodiment of the invention, an enzyme of sequence of SEQ ID No: 1 is such that:
X₁ is arginine or lysine, X₂ represents leucine, X₃ represents threonine and X₄ represents phenylalanine; or
X₁ is arginine or lysine, X₂ represents leucine, X₃ represents threonine and

X₄ represents asparagine.

In an embodiment of the invention, X₅ in sequence SEQ ID NO: 1 represents cysteine.

In a particular embodiment of the invention, an enzyme of sequence SEQ ID No: 1 is such that:
X₁ is arginine or lysine, X₂ represents leucine, X₃ represents threonine, X₄ represents phenylalanine and X₅ represents cysteine; or
X₁ is arginine or lysine, X₂ represents leucine, X₃ represents threonine, X₄ represents asparagine, and X₅ represents cysteine.

In an embodiment of the invention, X₆ in sequence SEQ ID NO: 1 represents an amino acid selected from the group consisting of leucine and asparagine. In a particular embodiment, X₆ in sequence SEQ ID NO: 1 is leucine.

In a particular embodiment, in sequence SEQ ID NO: 1, X₁ represents valine, X₂ represents lysine and X₆ represents asparagine.

In another embodiment, an enzyme of sequence SEQ ID No: 1 is such that:
X₁ is arginine or lysine, X₂ represents leucine, X₃ represents threonine, X₄ represents phenylalanine, X₅ represents cysteine and X₆ is leucine; or
X₁ is arginine or lysine, X₂ represents leucine, X₃ represents threonine, X₄ represents asparagine, X₅ represents cysteine and X₆ is leucine.

In an embodiment of the invention, X₇ in sequence SEQ ID NO: 1 represents alanine.

In a particular embodiment, an enzyme of sequence SEQ ID No: 1 is such that:
X₁ is arginine or lysine, X₂ represents leucine, X₃ represents threonine, X₄ represents phenylalanine, X₅ represents cysteine, X₆ is leucine and X₇ is alanine; or
X₁ is arginine or lysine, X₂ represents leucine, X₃ represents threonine, X₄ represents asparagine, X₅ represents cysteine, X₆ is leucine and X₇ is alanine.

In a particular embodiment, an enzyme of the invention is selected from the group consisting of:

(i) an enzyme of sequence SEQ ID NO: 1 wherein X₁ represents arginine; X₂ represents leucine; X₃ represents threonine; X₄ represents phenylalanine; X₅ represents cysteine; X₆ represents leucine; and X₇ represents alanine; (i.e. an enzyme of amino acid sequence SEQ ID NO: 2)

(ii) an enzyme of sequence SEQ ID NO: 1 wherein X₁ represents arginine; X₂ represents leucine; X₃ represents threonine; X₄ represents asparagine; X₅ represents cysteine; X₆ represents leucine; and X₇ represents alanine; (i.e. an enzyme of amino acid sequence SEQ ID NO: 3)

(iii) an enzyme of sequence SEQ ID NO: 1 wherein X₁ represents arginine; X₂ represents leucine; X₃ represents threonine; X₄ represents asparagine; X₅ represents cysteine; X₆ represents leucine; and X₇ represents alanine; (i.e. an enzyme of amino acid sequence SEQ ID NO: 4)

(iv) an enzyme of sequence SEQ ID NO: 1 wherein X₁ represents arginine; X₂ represents leucine; X₃ represents threonine; X₄ represents asparagine; X₅ represents cysteine; X₆ represents leucine; and X₇ represents leucine; (i.e. an enzyme of amino acid sequence SEQ ID NO: 5) and

(v) an enzyme of sequence SEQ ID NO: 1 wherein X₁ represents valine; X₂ represents lysine; X₃ represents threonine; X₄ represents asparagine; X₅ represents cysteine; X₆ represents asparagine; and X₇ represents alanine. (i.e. an enzyme of amino acid sequence SEQ ID NO: 6)

### Recombinant Yeast According to the Invention

The inventors have conceived recombinant microorganisms, and especially recombinant yeasts, having an increased ability of producing malonic semi-aldehyde, one of its salts, or a malonic semi-aldehyde derivative.

The present invention relates to recombinant yeasts having an increased malonic semi-aldehyde, malonic acid's salts, and malonic semi-aldehyde derivatives production and wherein this increased production is obtained through the introduction in said yeast of at least one nucleic acid sequence encoding an enzyme of the invention as described above.

A recombinant yeast according to the invention produces malonic semi-aldehyde, malonic acid's salts, and malonic semi-aldehyde derivatives with a higher yield than the parent yeast which does not contain the genetic modifications described above, i.e. which does not contain at least one nucleic acid sequence encoding an enzyme of sequence SEQ ID NO: 105, and in particular of sequence SEQ ID NO: 1, as defined above.

### Promoters

As it is disclosed herein, the expression of the nucleic acids of interest that have been genetically engineered for obtaining a recombinant yeast according to the invention comprise appropriate regulatory sequences that are functional in yeast cells, including in Saccharomyces cerevisiae.

As disclosed in the present specification, various promoters may be used for the desired expression of the coding sequences of interest, which include (i) constitutive strong promoters (also called strong promoters in the present text) and (ii) inducible promoters.

A list of yeast promoter with their relative activities in different media can be found in Keren et al. (2013) Molecular Systems Biology 9:701.

Promoters allowing the constitutive over-expression of a given gene, may be found in literature (Velculescu et al. (1997) Cell 88, 243-251).

Strong promoters more particularly interesting in the present invention may be selected from the group comprising:
pTDH3 (SEQ ID No 13),
pENO2 (SEQ ID No 14),
pTEF KI (SEQ ID No 15),
pTEF3 (SEQ ID No 16),
pTEF1 (SEQ ID No 17),
pADH1 (SEQ ID No 18),
pGMP1 (SEQ ID No 19),
pFBA1 (SEQ ID No 20),
pPDC1 (SEQ ID No 21),
pCCW12 (SEQ ID No 22), and
pGK1 (SEQ ID No 23).

In a particular embodiment, the strong promoter according to the invention is, independently, selected from the group consisting of pTDH3, pENO2, pTEF-KI, pTEF3, pTEF1, pADH1, pGMP1, pFBA1, pPDC1, pCCW12 and pGK1.

As previously mentioned, inducible promoters are promoters whose activity is controlled by the presence or absence of biotic or abiotic factors and also by the quantity of said factor. Accordingly, their activity will in particular be induced and thus increased when the quantity of a given factor increases or is increased. The quantity of said factor(s) in the culture medium of a recombinant yeast of the invention comprising inducible promoters can be decided and thus controlled by the man skilled in the art. For example, increasing the quantity of methionine in a culture medium of a recombinant yeast according to the invention comprising a pSAM4 promoter will induce and thus increase transcription of the gene under the control of this promoter.

In another example, reducing the quantity of copper in a culture medium of a recombinant yeast according to the invention comprising a pCTR1 promoter will lead to an induced, and thus an increased, transcription of the gene under the control of this promoter.

For this reason, the following promoters are referred to in the present text as being “inducible promoters”.

According to a first embodiment, inducible promoters more particularly interesting in the present invention may be selected from the group comprising promoters inducible with copper, promoters inducible with methionine and promoters inducible with threonine, and are in particular selected from the group consisting of:
pSAM4—methionine inducible (SEQ ID No 24),
pCUP1-1—copper inducible (SEQ ID No 25),
pCUP1.cgla—copper inducible (SEQ ID No 26),
pCUP1.sba—copper inducible (SEQ ID No 27),
pACU1—copper inducible (SEQ ID No 28),
pACU2—copper inducible (SEQ ID No 29),
pACU3p—copper inducible (SEQ ID No 30),
pACU4p—copper inducible (SEQ ID No 31),
pACU5—copper inducible (SEQ ID No 32),
pACU6—copper inducible (SEQ ID No 33),
pACU7—copper inducible (SEQ ID No 34),
pACU8—copper inducible (SEQ ID No 35),
pACU9—copper inducible (SEQ ID No 36),
pACU10p—copper inducible (SEQ ID No 37),
pACUl 1—copper inducible (SEQ ID No 38),
pACU12—copper inducible (SEQ ID No 39),
pACU13—copper inducible (SEQ ID No 40),
pACU14—copper inducible (SEQ ID No 41),
pACU15—copper inducible (SEQ ID No 42),
pGAL/CUP1p—copper inducible (SEQ ID No 43),
pCRS5—copper inducible (SEQ ID No 44), and
pCHA1—threonine inducible (SEQ ID No 45).

According to this embodiment, the inducible promoter according of the invention can in particular, independently, be selected from the group consisting of pSAM4, pCUP1-1, pCUP1.Cgla, pCUP1.Sba, pACUl, pACU2, pACU3p, pACU4p, pACU5, pACU6, pACU7, pACU8, pACU9, pACU10p, pACU11, pACU12, pACU13, pACU14, pACU15, pGAL/CUP1p, pCRS5, and pCHA1.

The activity of these promoters is thus induced by the increasing presence of methionine, copper or threonine as indicated above.

According to a second embodiment, inducible promoters according to the invention may be selected from the group comprising:
promoters inducible due to the absence of copper (i.e. promoter's activity is increased by the absence of copper. The fewer copper there is in the medium, the higher the activity of these promoters),
promoters inducible due to the absence of lysine (i.e. promoter's activity is increased by the absence of lysine. The fewer lysine there is in the medium, the higher the activity of these promoters) and
promoters inducible due to the absence of methionine (i.e. promoter's activity is increased by the absence of methionine. The fewer methionine there is in the medium, the higher the activity of these promoters),

and in particular are selected from the group consisting of:
pCTR1—copper inducible (SEQ ID No 46),
pCTR3—copper inducible (SEQ ID No 47),
pCUR1—copper inducible (SEQ ID No 48),
pCUR2—copper inducible (SEQ ID No 49),
pCUR3—copper inducible (SEQ ID No 50),
pCUR4—copper inducible (SEQ ID No 51),
pCUR5p—copper inducible (SEQ ID No 52),
pCUR6—copper inducible (SEQ ID No 53),
pCUR7—copper inducible (SEQ ID No 54),
pCUR8—copper inducible (SEQ ID No 55),
pCUR9—copper inducible (SEQ ID No 56),
pCUR10—copper inducible (SEQ ID No 57),
pCUR11—copper inducible (SEQ ID No 58),
pCUR12—copper inducible (SEQ ID No 59),
pCUR13—copper inducible (SEQ ID No 60),
pCUR14—copper inducible (SEQ ID No 61),
pCUR15—copper inducible (SEQ ID No 62),
pCUR16—copper inducible (SEQ ID No 63),
pCUR17—copper inducible (SEQ ID No 64),
pLYS1—lysine inducible (SEQ ID No 65),
pLYS4—lysine inducible (SEQ ID No 66),
pLYS9—lysine inducible (SEQ ID No 67),
pLYR1p—lysine inducible (SEQ ID No 68),
pLYR2p—lysine inducible (SEQ ID No 69),
pLYR3p—lysine inducible (SEQ ID No 70),
pLYR4p—lysine inducible (SEQ ID No 71),
pLYR5p—lysine inducible (SEQ ID No 72),
pLYR6p—lysine inducible (SEQ ID No 73),
pLYR7p—lysine inducible (SEQ ID No 74),
pLYR8—lysine inducible (SEQ ID No 75),
pLYR9—lysine inducible (SEQ ID No 76),
pLYR10—lysine inducible (SEQ ID No 77),
pLYR11—lysine inducible (SEQ ID No 78),
pMET17—methionine inducible (SEQ ID No 79),
pMET6—methionine inducible (SEQ ID No 80),
pMET14—methionine inducible (SEQ ID No 81),
pMET3—methionine inducible (SEQ ID No 82),
pSAM1—methionine inducible (SEQ ID No 83),
pSAM2—methionine inducible (SEQ ID No 84),
pMDH2—glucose inducible (SEQ ID No 85),
pJEN1—glucose inducible (SEQ ID No 86),
pICL1—glucose inducible (SEQ ID No 87),
pADH2—glucose inducible (SEQ ID No 88), and
pMLS1—glucose inducible (SEQ ID No 89).

According to this embodiment, the inducible promoter according to the invention can, independently, be selected from the group consisting of pCTR1, pCTR3, pCUR1, pCUR2, pCUR3, pCUR4, pCUR5p, pCUR6, pCUR7, pCUR8, pCUR9, pCUR10, pCUR11, pCUR12, pCUR13, pCUR14, pCUR15, pCUR16, pCUR17, pLYS1, pLYS4, pLYS9, pLYR1p, pLYR2p, pLYR3p, pLYR4p, pLYR5p, pLYR6p, pLYR7p, pLYR8, pLYR9, pLYR10, pLYR11, pMET17, pMET6, pMET14, pMET3, pSAM1, pSAM2, pMDH2, pJEN1, pICL1, pADH2 and pMLS1.

In a particular embodiment, inducible promoters according to the invention may be selected from the group comprising promoters inducible with copper, promoters inducible due to the absence of copper, promoters inducible due to the absence of glucose, promoters inducible due to the absence of lysine, promoters inducible with methionine, promoters inducible due to the absence of methionine and promoters inducible with threonine.

In a more particular embodiment, the inducible promoter according to the invention can, independently, be selected from the group consisting of pSAM4, pCUP1-1, pCUP1.Cgla, pCUP1.Sba, pACUl, pACU2, pACU3p, pACU4p, pACU5, pACU6, pACU7, pACU8, pACU9, pACU10p, pACU11, pACU12, pACU13, pACU14, pACU15, pGAL/CUP1p, pCRS5, pCHA1, pCTR1, pCTR3, pCUR1, pCUR2, pCUR3, pCUR4, pCUR5p, pCUR6, pCUR7, pCUR8, pCUR9, pCUR10, pCUR11, pCUR12, pCUR13, pCUR14, pCUR15, pCUR16, pCUR17, pLYS1, pLYS4, pLYS9, pLYR1p, pLYR2p, pLYR3p, pLYR4p, pLYR5p, pLYR6p, pLYR7p, pLYR8, pLYR9, pLYR10, pLYR11, pMET17, pMET6, pMET14, pMET3, pSAM1, pSAM2, pMDH2, pJEN1, pICL1, pADH2 and pMLS1.

More particularly, said promoters, identical or different, may be preferably characterized by a sequence of nucleic acid selected from the group consisting of sequences having at least 80% identity with sequences SEQ ID NO: 13 to 89.

Synthetic promoters as described in Blazeck & Alper (2013) Biotechnol. J. 8 46-58 can also be used.

The strong and inductible or repressible promoters of the invention can originate from any organism from the Saccharomycetes class and can in particular originate, independently, from an organism selected from the group consisting of Saccharomyces cerevisiae, Saccharomyces boulardii, Saccharomyces castelii, Saccharomyces bayanus, Saccharomyces arboricola, Saccharomyces kudriavzevii, Ashbya gossypii, Kluveromyces lactis, Pichia pastoris, Candida glabrata, Candida tropicalis, Debaryomyces castelii, Yarrowia lipolitica and Cyberlindnera jadinii.

The strong, weak and inductible promoters of the invention can preferably originate from an organism selected from the group consisting of Saccharomyces cerevisiae, Saccharomyces castelii, Saccharomyces bayanus, Saccharomyces arboricola, Saccharomyces kudriavzevii and Kluveromyces lactis.

### Terminators

The expression of the genes of interest that have been genetically engineered for obtaining a recombinant yeast according to the invention comprise appropriate transcription terminator sequences that are functional in yeast cells, including in Saccharomyces cerevisiae.

Said transcription terminators, identical or different, may be found in literature Yamanishi et al., (2013) ACS synthetic biology 2, 337-347.

Terminators of the present invention can for example be selected from the group comprising:
tTDH2 from the gene coding for Glyceraldehyde-3-phosphate dehydrogenase, isozyme 2 (TDH2 gene=Sequence SEQ ID No 90),
tCYC1 (=Sequence SEQ ID No 91),
tTDH3 (=Sequence SEQ ID No 92),
tADH1 from gene coding for the alcohol dehydrogenase (ADH1 gene=Sequence SEQ ID No 93),
tADH2 from gene coding for the alcohol dehydrogenase (ADH2 gene=Sequence SEQ ID No 94),
tTPI1 from the gene encoding for the Triose Phosphate Isomerase (TPI1 gene=Sequence SEQ ID No 95),
tMET17 from the gene encoding for the O-acetyl homoserine-O-acetyl serine sulfhydrylase (Met17 gene=Sequence SEQ ID No 96),
tENO2 from the gene coding for Enolase II (ENO2 gene=Sequence SEQ ID No 97),
tMET3 (=Sequence SEQ ID No 98),
tPGK1 from the gene encoding for the 3-phosphoglycerate kinase (PGK1 gene=Sequence SEQ ID No 99),
tDIT1 (=Sequence SEQ ID No 100)
tRPL3 (=Sequence SEQ ID No 101)
tRPL41B (=Sequence SEQ ID No 102)
tRPL15A (=Sequence SEQ ID No 103)
tIDP1 (=Sequence SEQ ID No 104)

More particularly, said terminator, identical or different, may be preferably characterized by a sequence of nucleic acid selected from the group consisting of sequences having at least 80% identity with sequences SEQ ID NO: 90 to 104.

### Recombinant Yeast

Generally, yeast can grow rapidly and can be cultivated at higher density as compared with bacteria, and does not require an aseptic environment in the industrial setting. Furthermore, yeast cells can be more easily separated from the culture medium compared to bacterial cells, greatly simplifying the process for product extraction and purification.

Preferentially, the yeast of the invention may be selected among the genus Saccharomyces, Candida Ashbya, Dekkera, Pichia (Hansenula), Debaryomyces, Clavispora, Lodderomyces, Yarrowia, Zigosaccharomyces, Schizosaccharomyces, Torulaspora, Kluyveromyces, Brettanomycces, Cryptococcus or Malassezia.

More preferentially, the yeast may be Crabtree positive yeast of genus of Saccharomyces, Dekkera, Schizosaccharomyces, Kluyveromyces, Torulaspora Zigosaccharomyces, or Brettanomycces

More preferentially, the yeast may be from the species Saccharomyces cerevisiae, Saccharomyces boulardii, Saccharomyces douglasii, Saccharomyces bayanus or. or Zigosaccharomyces bailii, Schizosaccharomyces pombe, Dekkera brucelensis, Dekkera intermedia, Brettanomycces custersii, Brettanomycces intermedius, Kluyveromyces themotolerens, Torulaspora globosa, Torulaspora glabrata

More preferentially, the recombinant yeast may belong to the Saccharomyces genus, and preferably to the Saccharomyces cerevisiae species.

As above-mentioned, a recombinant yeast according to the invention is able to decarboxylate oxaloacetate into malonic semi-aldehyde due to the insertion of at least one nucleic acid of sequence SEQ ID NO: 105, and in particular of sequence SEQ ID NO: 1.

Methods implemented to insert a specific DNA construct within a gene belong to the general knowledge of a man skilled in the art. A related method is described in more details in the herein after examples.

### Culture Conditions

The present invention also relates to the use of a recombinant yeast such as above-defined, for the production of malonic semi-aldehyde, one of its salts, or a malonic semi-aldehyde derivative.

The present invention further relates to a method for producing malonic semi-aldehyde, one of its salts, or a malonic semi-aldehyde derivative, said method comprising the steps of:

(a) culturing a recombinant yeast as defined in the present text in a culture medium; and

(b) recovering the malonic semi-aldehyde, one of its salts, or a malonic semi-aldehyde derivative from said culture medium.

Typically, microorganisms of the invention are grown at a temperature in the range of about 20° C. to about 37° C., preferably at a temperature ranging from 27 to 34° C., in an appropriate culture medium.

When the recombinant yeast according to the invention belongs to the S. cerevisiae species, the temperature may advantageously range from 27 to 34° C., in an appropriate culture medium.

Suitable growth media for yeast are common commercially prepared media such as broth that includes yeast nitrogen base, ammonium sulfate, and dextrose as the carbon/energy source) or YPD Medium, a blend of peptone, yeast extract, and dextrose in optimal proportions for growing most. Other defined or synthetic growth media may also be used and the appropriate medium for growth of the particular microorganism will be known by one skilled in the art of microbiology or fermentation science.

The term “appropriate culture medium” is above-defined.

Examples of known culture media for a recombinant yeast according to the present invention are known to the person skilled in the art, and are presented in the following publication D. Burke et al., Methods in yeast Genetics—A cold spring harbor laboratory course Manual (2000).

Suitable pH ranges for the fermentation may be between pH 3.0 to pH 7.5, where pH 4.5 to pH 6.5 is preferred as the initial condition.

Fermentations may be performed under aerobic conditions or micro-aerobic conditions.

The amount of product in the fermentation medium can be determined using a number of methods known in the art, for example, high performance liquid chromatography (HPLC) or gas chromatography (GC).

The present process may employ a batch method of fermentation. A classical batch fermentation is a closed system where the composition of the medium is set at the beginning of the fermentation and not subject to artificial alterations during the fermentation. Thus, at the beginning of the fermentation, the medium is inoculated with the desired organism or organisms, and fermentation is permitted to occur without adding anything to the system. Typically, however, a “batch” fermentation is batch with respect to the addition of carbon source and attempts are often made at controlling factors such as temperature, pH and oxygen concentration. In batch systems, the metabolite and biomass compositions of the system change constantly up to the time when the fermentation is stopped. Within batch cultures cells progress through a static lag phase to a high growth log phase and finally to a stationary phase where growth rate is diminished or halted. If untreated, cells in the stationary phase will eventually die. Cells in log phase generally are responsible for the bulk of production of end product or intermediate.

A Fed-Batch system may also be used in the present invention. A Fed-Batch system is similar to a typical batch system with the exception that the carbon source substrate is added in increments as the fermentation progresses. Fed-Batch systems are useful when catabolite repression (e.g. glucose repression) is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂.

Fermentations are common and well known in the art and examples may be found in Sunderland et al., (1992), herein incorporated by reference. Although the present invention is performed in batch mode it is contemplated that the method would be adaptable to continuous fermentation.

Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to vary. In other systems a number of factors affecting growth can be altered continuously while the cell concentration, measured by media turbidity, is kept constant. Continuous systems strive to maintain steady state growth conditions and thus the cell loss due to the medium being drawn off must be balanced against the cell growth rate in the fermentation. Methods of modulating nutrients and growth factors for continuous fermentation processes as well as techniques for maximizing the rate of product formation are well known in the art of industrial microbiology.

It is contemplated that the present invention may be practiced using either batch, fed-batch or continuous processes and that any known mode of fermentation would be suitable. Additionally, it is contemplated that cells may be immobilized on a substrate as whole cell catalysts and subjected to fermentation conditions for production.

In order to still improve the production of malonic semi-aldehyde, one of its salts, or a malonic semi-aldehyde derivative, a particular embodiment may consist of culturing the recombinant yeast cells in an appropriate culture medium, such as above-mentioned, wherein the said culture medium comprises an optimal amount of carbon source, especially glucose.

Preferably, the cells are cultured in such an optimal culture medium during only a part of the whole culture duration. In some embodiments, the yeast cells are incubated in the said optimal culture medium 10 hours or more after initiation of the culture, which encompasses 11, 12, 13, 14, 15 or 16 hours or more after initiation of the culture.

Preferably, the cells are cultured in such an optimal culture medium during a time period ranging from 5 hours to 15 hours, which includes from 6 hours to 10 hours, e.g. 8 hours after initiation of the culture.

In preferred embodiments, the carbon source comprised in said optimal culture medium consists of glucose. In preferred embodiments, the said optimal culture medium comprises 12% w/w or more glucose, including 15% w/w or more glucose. In preferred embodiments, the said optimal culture medium comprises at most 40% w/w glucose, which includes at most 35% w/w glucose.

Thus, in the preferred embodiments described above, a method for producing malonic semi-aldehyde, one of its salts, or a malonic semi-aldehyde derivative according to the invention may further comprise, between steps (a) and (c), an intermediate step (b) consisting of cultivating the yeast cells in the said optimal culture medium.

### Purification of Malonic Semi-Aldehyde, One of its Salts, or a Malonic Semi-Aldehyde Derivative

According to a specific aspect of the invention, the fermentative production of malonic semi-aldehyde, one of its salts, or a malonic semi-aldehyde derivative comprises a step of isolation of the malonic semi-aldehyde, of one of its salts, or of a malonic semi-aldehyde derivative from the culture medium. Recovering the malonic semi-aldehyde, one of its salts, or the malonic semi-aldehyde derivative from the culture medium is a routine task for a man skilled in the art. It may be achieved by a number of techniques well known in the art including but not limiting to distillation, gas-stripping, pervaporation, selective precipitation or liquid extraction. The expert in the field knows how to adapt parameters of each technique dependent on the characteristics of the material to be separated.

The yeast as model of microorganism in the present invention has been retained in that the synthesized malonic semi-aldehyde, salts of malonic semi-aldehyde, or malonic semi-aldehyde derivatives can be entirely exported outside the cells, thus simplifying the purification process.

The synthesized malonic semi-aldehyde, one of its salts, or malonic semi-aldehyde derivatives may be collected by distillation. Distillation may involve an optional component different from the culture medium in order to facilitate the isolation of malonic semi-aldehyde, one of its salts, or malonic semi-aldehyde derivative by forming azeotrope and notably with water. This optional component is an organic solvent such as cyclohexane, pentane, butanol, benzene, toluene, trichloroethylene, octane, diethylether or a mixture thereof.

Gas stripping is achieved with a stripping gas chosen among helium, argon, carbon dioxide, hydrogen, nitrogen or mixture thereof.

Liquid extraction is achieved with organic solvent as the hydrophobic phase such as pentane, hexane, heptane or dodecane.

### Malonic Semi-Aldehyde, One of its Salts, and Malonic Semi-Aldehyde Derivatives

Malonic semi-aldehyde, also known as 3-Oxopropanoic acid, is a compound having the following structure:

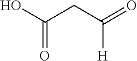

This compound may exist in the form of a base or of a salt. These salts also form part of the invention.

Such a salt can for example be malonate semi-aldehyde having the following structure:

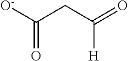

Organic cation salts, such as ammonium, sodium, potassium, phosphonium or sulfonium salts, may also be concerned.

Malonic semi-aldehyde derivatives according to the invention are compounds that can be obtained from malonic semi-aldehyde, or from one of its salts, after modification by at least one enzyme naturally and/or artificially present in the microorganism producing the malonic semi-aldehyde, or one of its salts, according to the invention, in particular in the yeast producing the malonic semi-aldehyde, or one of its salts, according to the invention.

Examples of such derivatives of malonate semi-aldehyde can for example be propanol, propanal, acrylic acid, acrylyl-CoA, isopropanol, acrylate, proprionate, propyonyl-CoA, 3-hydroxypropionate, 3 hydroxypropyl-CoA, 3-hydroxy-3-methyl-butyrate, phloroglucinol, flavonoids, cannabinoids, farnesyl-PP, squalene and derivatives of these pathways.

In a particular embodiment, derivatives of malonate semi-aldehyde are selected from the group consisting of propanol, propanal, acrylic acid, acrylyl-CoA, isopropanol, acrylate, proprionate, propyonyl-CoA, 3-hydroxypropionate, 3 hydroxypropyl-CoA, 3-hydroxy-3-methyl-butyrate, phloroglucinol, flavonoids, cannabinoids, farnesyl-PP and squalene.

In a particular embodiment, derivatives of malonate semi-aldehyde are selected from the group consisting of 3-hydroxypropionate, propanol, acrylic acid, isopropanol, acrylate and proprionate.

These compounds can for example be obtained through transformation of malonic semi-aldehyde with various endogeneous or exogeneous enzymes in the yeast according to the invention, as represented here-after.

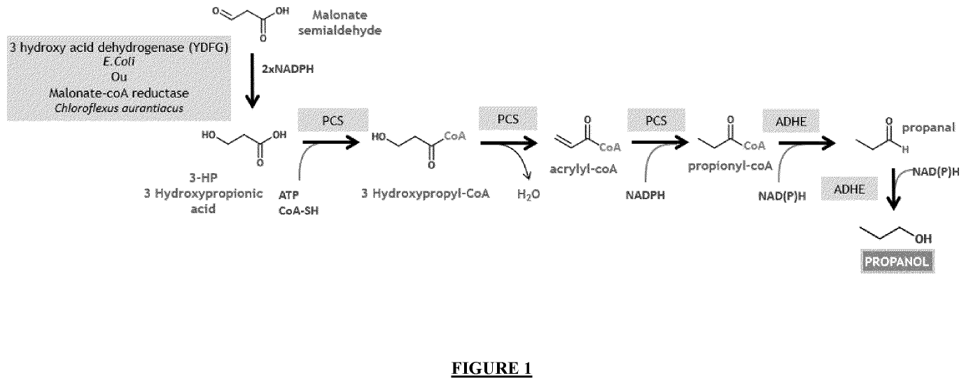

3-hydroxypropionate, 3-hydroxypropionate, Acrylyl-CoA, Propyonyl-CoA, propanal and propanol can for example be obtained from malonic semi-aldehyde or from one of its salts through the steps illustrated in FIG. 1.

PCS represents a propionyl-coA synthase, such as for example the PCS of Chloroflexus aggregans, Roseiflexus castenholzii or Chloroflexus aurantiacus.

ADHE represents an alcohol dehydrogenase E. It can for example be the ADHE from Clostridium beijerinckii or Clostridium arbusti.

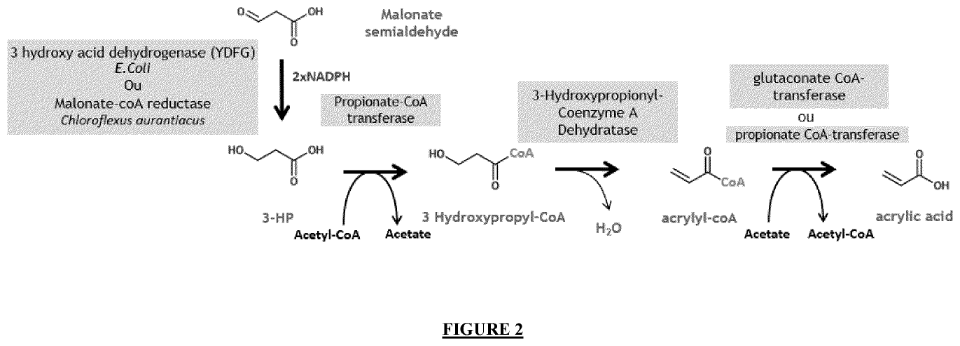

Acrylic acid and acrylate can for example be obtained from malonic semi-aldehyde or from one of its salts through the steps illustrated in FIG. 2.

Malonate semi-aldehyde can be transformed into acetyl-coA by a malonate-semi-aldehyde dehydrogenase (acetylating) (E.C 1.2.1.18) as for example KES23460 from Pseudomonas putida described in Wilding et al. (2016) Appl. Env. Microbiology, 82, 3846-3856. Acetyl-coA is then a starting point to produce isopropanol as described in Tamakawa et al. Appl Microbiol Biotechnol (2013) 97:6231-6239.

Propionyl-CoA is obtained from malonate semi-aldehyde as described above for the proposed propanol pathway. Propionyl-coA can then transformed into propionate through the successive catalysis of a PhosphoTrans Acetylase (E.C 2.3.1.8) and a Acetate Kinase (E.C 2.7.2.1) as described in Erbilgin et al. (2016) PLoS Biol 14(3): e1002399.doi:10.1371/journal.pbio.1002399 and Reinscheid et al. (1999) Microbiology 145, 503-513.

Malonic semi-aldehyde can also be transformed into malonyl-coA by a malonyl-CoA reductase (E.C 1.2.1.75) as described in Alber et al. (2006) Journal of bacteriology 188, 8551-8559. Malonyl-coA is then the starting point to synthetize phloroglucinol and derivatives using a phloroglucinol synthase (E.C.2.3.1.253) as described in Yang and Cao (2012) Appl Microbiol Biotechnol 93:487-495.

Malonyl-coA is a major building block and often a bottelneck required for flavonoids biosynthesis (Johnson et al. (2017) Metabolic Engineering 44: 253-264). Malonic semi-aldehyde can be transformed into malonyl-coA by a malonyl-CoA reductase (E.C 1.2.1.75) as described in Alber et al. (2006) Journal of bacteriology 188, 8551-8559. Malonyl-coA can then be used to fuel flavonoids synthesis as described in Batra, Priya, et Anil K. Sharma. (2013) 3 Biotech 6: 439-59. https://doi.org/10.1007/s13205-013-0117-5; and in Mou, et al. (2015) PLoS ONE 10 (6) https://doi.org/10.1371/journal.pone.0129598.

Acetyl-CoA is also a major building block and often a bottelneck required for farnesyl-PP and derivatives biosynthesis, as for example squalene. As mentioned above, malonate semi-aldehyde can be transformed into acetyl-coA by a malonate semi-aldehyde dehydrogenase (acetylating) (E.C 1.2.1.18) as for example KES23460 from Pseudomonas putida described in Wilding et al. (2016) Appl. Env. Microbiology, 82, 3846-3856. Acetyl-coA is then a starting point to produce Farnesyl coA and derivatives as described in Wang, J.; Li, Y.; Liu, D. Cloning and Characterization of Farnesyl Diphosphate Synthase Gene Involved in Triterpenoids Biosynthesis from Poria cocos. Int. J. Mol. Sci. 2014, 15, 22188-22202.

Cannabinoids can for example be obtained from malonic semi-aldehyde or from one of its salts from acetyl-coA which is the precursor of all cannabinoids. Malonate semi-aldehyde can be transformed into acetyl-coA by a malonate-semi-aldehyde dehydrogenase (acetylating) (E.C 1.2.1.18) as for example KES23460 from Pseudomonas putida described in Wilding et al.. Acetyl-coA is then a starting point to produce cannabinoids as described in Carvalho et al. (2017) FEMS Yeast Research, 17, fox037. doi: 10.1093/femsyr/fox037.

3-hydroxy-3-methyl-butyrate can for example be obtained from malonic semi-aldehyde or from one of its salts as follows. Malonate semi-aldehyde can, as mentioned above, be transformed into acetyl-coA by a malonate-semi-aldehyde dehydrogenase (acetylating) (E.C 1.2.1.18) as for example KES23460 from Pseudomonas putida described in Wilding et al.. Acetyl-coA is then the starting point for 3-hydroxy-3-methyl-butyrate biosynthesis as described in Gogerty and Bobic (2010) Appl Microbiol Biotechnol 76: 8004-8010.

Accordingly, the recombinant yeast according to the invention can comprise one or more nucleic acid genes encoding the enzymes mentioned above in order to obtain the malonic semi-aldehyde derivatives of interest. These one or more nucleic acid genes encoding the enzymes performing the necessary transformations of malonic semi-aldehyde, or one of its salts, to the malonic semi-aldehyde derivative of interest can be naturally present in the yeast (endogenous) and/or can be incorporated into the yeast as transgenes according to methods well known to the man skilled in the art.

Accordingly, in a particular embodiment, a recombinant yeast according to the invention comprise, in addition to a nucleic acid gene encoding an enzyme able to catalyze the decarboxylation of oxaloacetate into malonic semi-aldehyde as defined above, at least one nucleic acid encoding a 3-hydroxy acid dehydrogenase and/or a malonate-coA reductase.

The 3-hydroxy acid dehydrogenase and/or malonate-coA reductase according to this embodiment can be the 3-hydroxy acid dehydrogenase (YDFG) of E. coli (BAA15241.1) or the malonate-coA reductase from Chloroflexus aurantiacus (SEQ ID NO: 7).

In another embodiment, a recombinant yeast according to the invention comprise, in addition to a nucleic acid gene encoding an enzyme able to catalyze the decarboxylation of oxaloacetate into malonic semi-aldehyde as defined above, (i) at least one nucleic acid encoding a 3-hydroxy acid dehydrogenase or a malonate-coA reductase and (ii) at least one nucleic acid encoding a propionyl-coA synthase.

The 3-hydroxy acid dehydrogenase and/or malonate-coA reductase according to this embodiment can be the 3-hydroxy acid dehydrogenase (YDFG) of E. coli (BAA15241.1) or the malonate-coA reductase from Chloroflexus aurantiacus (SEQ ID NO: 7). The propionyl-coA synthase (PCS) according to this embodiment can for example be the PCS of Chloroflexus aggregans (SEQ ID NO: 8), Roseiflexus castenholzii (SEQ ID NO: 9) or Chloroflexus aurantiacus (SEQ ID NO: 10).

In another embodiment, a recombinant yeast according to the invention comprise, in addition to a nucleic acid gene encoding an enzyme able to catalyze the decarboxylation of oxaloacetate into malonic semi-aldehyde as defined above, (i) at least one nucleic acid encoding a 3-hydroxy acid dehydrogenase or a malonate-coA reductase, (ii) at least one nucleic acid encoding a propionyl-coA synthase and (iii) at least one alcohol dehydrogenase E (ADHE).

The 3-hydroxy acid dehydrogenase and/or malonate-coA reductase according to this embodiment can be the 3-hydroxy acid dehydrogenase (YDFG) of E. coli (BAA15241.1) or the malonate-coA reductase from Chloroflexus aurantiacus (SEQ ID NO: 7). The propionyl-coA synthase (PCS) according to this embodiment can for example be the PCS of Chloroflexus aggregans (SEQ ID NO: 8), Roseiflexus castenholzii (SEQ ID NO: 9) or Chloroflexus aurantiacus (SEQ ID NO: 10). The ADHE according to this embodiment can for example be the ADHE from Clostridium beijerinckii (SEQ IS NO: 11) or from Clostridium arbusti (SEQ ID NO: 12).

According to another embodiment, a recombinant yeast according to the invention comprise, in addition to a nucleic acid gene encoding an enzyme able to catalyze the decarboxylation of oxaloacetate into malonic semi-aldehyde as defined above, (i) at least one nucleic acid encoding a 3-hydroxy acid dehydrogenase or a malonate-coA reductase and (ii) at least one nucleic acid encoding a propionate-coA transferase.

A propionate-coA transferase appropriate according to the invention can for example be the enzyme with the reference E.C.2.8.3.1.

The 3-hydroxy acid dehydrogenase and/or malonate-coA reductase according to this embodiment can be the 3-hydroxy acid dehydrogenase (YDFG) of E. coli (BAA15241.1) or the malonate-coA reductase from Chloroflexus aurantiacus (SEQ ID NO: 7). The propionate-coA transferase according to this embodiment can be the propionate-coA transferase of Cupriavidus necator (EC 2.8.3.1).

In another embodiment, a recombinant yeast according to the invention comprise, in addition to a nucleic acid gene encoding an enzyme able to catalyze the decarboxylation of oxaloacetate into malonic semi-aldehyde as defined above, (i) at least one nucleic acid encoding a 3-hydroxy acid dehydrogenase or a malonate-coA reductase, (ii) at least one nucleic acid encoding a propionate-coA transferase and (iii) at least one 3-hydroxypropionyl coenzyme A dehydratase.

The 3-hydroxy acid dehydrogenase and/or malonate-coA reductase according to this embodiment can be the 3-hydroxy acid dehydrogenase (YDFG) of E. coli (BAA15241.1) or the malonate-coA reductase from Chloroflexus aurantiacus (SEQ ID NO: 7). The propionate-coA transferase according to this embodiment can be the propionate-coA transferase of Cupriavidus necator (EC 2.8.3.1). The 3-hydroxypropionyl coenzyme A dehydratase according to this embodiment can be the 3-hydroxypropionyl coenzyme A dehydratase of Metallosphaera sedula (EC 4.2.1.116).

The terms “between . . . and . . . ” and “ranging from . . . to . . . ” should be understood as being inclusive of the limits, unless otherwise specified.

The examples and figures which follow are presented by way of illustration and without implied limitation of the invention.

### EXAMPLES

### Example 1a: Protocol for Making a Recombinant Saccharomyces cerevisiae Strain According to the Invention

All the hereinafter implemented recombinant Saccharomyces cerevisiae strains were constructed from standard strains using standard yeast molecular genetics procedure (Methods in yeast Genetics—A cold spring harbor laboratory course Manual (2000) by D. Burke, D. Dawson, T. Stearns CSHL Press).

The following-mentioned genes were integrated in recombinant yeast using the ability of yeast to efficiently recombine free DNA ends which have sequence homology.

More particularly, the coding sequences to be cloned were artificially synthetized.

For heterologous sequences (non-yeast), the nucleic sequences were modified in order to obtain a synonymous coding sequence using the yeast codon usage. Using restriction enzyme and classical cloning technology, each synthetic sequence was cloned in between a transcription promoter and a transcription terminator. Each promoter sequence is preceded by a 50 to 200 nucleotide sequence homologous to the sequence of the terminator of the upstream gene. Similarly, the terminator of each gene (a gene comprising the promoter-coding sequence-terminator) is followed by sequences homologous to the gene immediately following. So that each of the unit to be integrated have a 50-200 nucleotide overlap with both the unit upstream and the unit downstream. For the first unit, the promoter is preceded by 50-200 nucleotides homologous to the yeast chromosome nucleotide for the locus in which it will be integrated. Similarly, for the last unit, the terminator is followed by 50-200 nucleotides homologous to the yeast chromosome nucleotide for the locus in which it will be integrated.

Each unit are then PCR amplified from the plasmids constructs, yielding X unit of linear DNA having overlapping sequences. At least one of this gene is an auxotrophic marker, in order to select for recombination event. All the linear fragments are transformed in the yeast at once, and recombinant yeast are selected for the auxotrophy related to the marker used. The integrity of the sequence is then verified by PCR and sequencing.

On the basis of the enzymes' sequences disclosed herein, the man skilled in the art knows how to obtain them through well known synthetic biological synthesis technics, for example illustrated in Tian J. et al., Mol. Biosyst. 2009; 5(7):714-22.

### Example 2: Results Concerning the Use of Enzymes of Sequence SEQ ID NO: 105 According to the Invention

More particularly, in the present invention, the recombinant yeasts are mutant yeasts (Δfms1) which are impaired for β-alanine synthesis. As a consequence, the yeast is auxotroph for panthotenate and cannot grow on a medium deprived of pantothenate. In the same yeasts is expressed PYD4 from Lachancea kluyveri, a gene which encodes a β-alanine aminotransferase activity absent from Saccharomyces cerevisiae.

This enzyme is able to transform Malonyl-semi-aldehyde into β-alanine (Andersen et al. 2007 FEBS Journal 274, 1804-1817). This yeast then lacks an activity able to produce malonyl semi-aldehyde to be able to grow in absence of pantothenate in the medium.

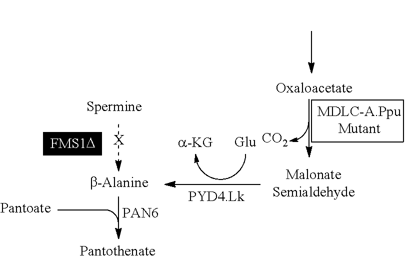

This yeast is still unable to grow on a pantothenate free medium upon expression of the benzoylformate decarboxylase from P. putida. Indeed, benzoyl formate decarboxylase is not able to catalyze the transformation of oxaloacetate into malonic semi-aldehyde.

A. The following enzymes have been tested in vivo, in separate yeasts.

Enzyme No 1:
(SEQ ID NO: 2)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYFCAAGGLGFALPAAIGVQLAEPERQVIAVIGDGSANYSISALWTAAQYNIPTIFVIMNNG
TYGALRWFAGVLEAENVPGLDVPGIDFRALAKGYGVQALKADNLEQLKGSLQEALSAKGPVLIEVSTV
SPVK
Enzyme No 2:
(SEQ ID NO: 3)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYNCAAGGLGFALPAAIGVQLAEPERQVIAVIGDGSANYSISALWTAAQYNIPTIFVIMNNG
TYGALRWFAGVLEAENVPGLDVPGIDFRALAKGYGVQALKADNLEQLKGSLQEALSAKGPVLIEVSTV
SPVK
Enzyme No 3:
(SEQ ID NO: 4)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEAKLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYNCAAGGLGFALPAAIGVQLAEPERQVIAVIGDGSANYSISALWTAAQYNIPTIFVIMNNG
TYGALRWFAGVLEAENVPGLDVPGIDFRALAKGYGVQALKADNLEQLKGSLQEALSAKGPVLIEVSTV
SPVK
Enzyme No 4:
(SEQ ID NO: 5)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYNCAAGGLGFALPAAIGVQLAEPERQVIAVIGDGSANYSISALWTAAQYNIPTIFVIMNNG
TYGLLRWFAGVLEAENVPGLDVPGIDFRALAKGYGVQALKADNLEQLKGSLQEALSAKGPVLIEVSTV
SPVK
Enzyme No 5:
(SEQ ID NO: 6)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEAVKTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYNCAAGGNGFALPAAIGVQLAEPERQVIAVIGDGSANYSISALWTAAQYNIPTIFVIMNN
GTYGALRWFAGVLEAENVPGLDVPGIDFRALAKGYGVQALKADNLEQLKGSLQEALSAKGPVLIEVST
VSPVK
Enzyme No 6:
(SEQ ID NO: 106)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYLCAAGGLGFALPAAIGVQLAEPERQVIAVIGDGSANYSISALWTAAQYNVPTIFVIMNN
GTYGTLRWFAGVLEAENVPGLDVPGIDFRALAKGYGVQALKADNLEQLKGSLQEALSAKGPVLIEVST
VSPVK
Enzyme No 7:
(SEQ ID NO: 107)
MASVHGTTYELLRRQGIDIVFGNPGSNELPFLKDFPEDFRYILALQEACVVGIADGYAQA
SRKPAFINLHSAAGTGNAMGALSNAWNSHSPLIVTAGQQTRAMIGVEARLTNVDAANLPRPLVKWS
YEPASAAEVPHAMSRAIHMASMAPQGPVYLSVPYDDWDKDADPQSHHLFDRHVSSSVRLNDQDL
DILVKALNSASNPAIVLGPDVDAANANADCVMLAERLKAPVWVAPSAPRCPFPTRHPCFRGLMPAGI
AAISQLLEGHDVVLVIGAPVFRYRQYDPGQYLKPGTRLISVTCDPLEAARAPMGDAIVADIGAMASAL
ANLVEESSRQLPTAAPEPAKVDQDAGRLHPETVFDTLNDMAPENAIYLNESTSTTAQMWQRLDMR
NPGSYYFCAAGGLGFALPAAIGVQLAEPGRQVIAVIGDGSANYSISALWTAAQYNIPTIFVIMNNGTY
GLLRWSAGVLGAENVPGLDVPGIDFRALAKGYGVQALKADNLEQLKGSLQEALSAKGPVLIEVSTVSP
VK
Enzyme No 8:
(SEQ ID NO: 108)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLM
AGIAAISQLLEGHDVVLVIGAPVFRYRQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYFCAAGGLGFALPAAIGVQLAEPERQVIAVIGDGSANYSISALWTAAQYNIPTIFVIMNNG
TYGGLRWFAGVLEAENVPGLDVPGIDFRALAKGYGVQALKADNLEQLKGSLQEALSAKGPVLIEVSTV
SPVK
Enzyme No 9:
(SEQ ID NO: 109)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYFCAAGGLGFALPAAIGVQLAEPERQVIAVIGDGSANYSISALWTAAQYNIPTIFVIMNNG
TYGTLRWFAGVLEAENVPGLDVPGIDFRALAKGYGVQALKADNLEQLKGSLQEALSAKGPVLIEVSTV
SPVK
Enzyme No 10:
(SEQ ID NO: 110)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYACAAGGLGFALPAAIGVQLAEPERQVIAVIGDGSANYSISALWTAAQYNIPTIFVIMNNG
TYGALRWFAGVLEAENVPGLDVPGIDFRALAKGYGVQALKADNLEQLKGSLQEALSAKGPVLIEVSTV
SPVK
Enzyme No 11:
(SEQ ID NO: 111)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYFCAAGGLGFALPAAIGVQLAEPERQVIAVIGDGSANYSISALWTAAQYNIPTIFVIMNNG
TYGLLRWFAGVLEAENVPGLDVPGIDFRALAKGYGVQALKADNLEQLKGSLQEALSAKGPVLIEVSTV
SPVK
Enzyme No 12:
(SEQ ID NO: 112)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYRQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYFCAAGGLGFALPAAIGVQLAEPERQVIAVIGDGSANYSISALWTAAQYNIPTIFVIMNNG
TYGALRWFAGVLEAENVPGLDVPGIDFRALAKGYGVQALKADNLEQLKGSLQEALSAKGPVLIEVSTV
SPVK
Enzyme No 13:
(SEQ ID NO: 113)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYRQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYFCAAGGLGFALPAAIGVQLAEPERQVIAVIGDGSANYSISALWTAAQYNIPTIFVIMNNG
TYGTLRWFAGVLEAENVPGLDVPGIDFRALAKGYGVQALKADNLEQLKGSLQEALSAKGPVLIEVSTV
SPVK
Enzyme No 14:
(SEQ ID NO: 114)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYFCAAGGLGFALPAAIGVQLAEPERQVIAVIGDGSANYSISALWTAAQYNIPTIFVIMNNG
TYGALRWFAGVLEAENVPGLDVPGIDFRALAKGYGVQALKADNLEQLKGSLQEALSAKGPVLIEVSTV
SPVK
Enzyme No 15:
(SEQ ID NO: 115)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYFCAAGGLGFALPAAIGVQLAEPERQVIAVIGDGSANYSISALWTAAQYNIPTIFVIMNNG
TYGLLRWFAGVLEAENVPGLDVPGIDFRALAKGYGVQALKADNLEQLKGSLQEALSAKGPVLIEVSTV
SPVK
Enzyme No 16:
(SEQ ID NO: 116)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYLCAAGGLGFALPAAIGVQLAEPERQVIAVIGDGSANYSISALWTAAQYNVPTIFVIMNN
GTYGLLRWFAGVLEAENVPGLDVPGIDFRALAKGYGVQALKADNLEQLKGSLQEALSAKGPVLIEVST
VSPVK
Enzyme No 17:
(SEQ ID NO: 117)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYLCAAGGLGFALPAAIGVQLAEPERQVIAVIGDGSANYSISALWTAAQYNVPTIFVIMNN
GTYGGLRWFAGVLEAENVPGLDVPGIDFRALAKGYGVQALKADNLEQLKGSLQEALSAKGPVLIEVST
VSPVK
Enzyme No 18:
(SEQ ID NO: 118)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYLCAAGGLGFALPAAIGVQLAEPERQVIAVIGDGSANYSISALWTAAQYNVPTIFVIMNN
GTYGTLRWSAGVLEAENVPGLDVPGIDFRALAKGYGVQALKADNLEQLKGSLQEALSAKGPVLIEVST
VSPVK
Enzyme No 19:
(SEQ ID NO: 119)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYLCAAGGLGFALPAAIGVQLAEPGRQVIAVIGDGSANYSISALWTAAQYNVPTIFVIMNN
GTYGTLRWSAGVLGAENVPGLDVPGIDFRALAKGYGVQALKADNLEQLKGSLQEALSAKGPVLIEVST
VSPVK
Enzyme No 20:
(SEQ ID NO: 120)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYLCAAGGLGFALPAAIGVQLAEPGRQVIAVIGDGSANYSISALWTAAQYNIPTIFVIMNNG
TYGTLRWSAGVLGAENVPGLDVPGIDFRALAKGYGVQALKADNLEQLKGSLQEALSAKGPVLIEVSTV
SPVK
Enzyme No 21:
(SEQ ID NO: 121)
MASVHGTTYELLRRQGIDIVFGNPGSNELPFLKDFPEDFRYILALQEACVVGIADGYAQA
SRKPAFINLHSAAGTGNAMGALSNAWNSHSPLIVTAGQQTRAMIGVEARLTNVDAANLPRPLVKWS
YEPASAAEVPHAMSRAIHMASMAPQGPVYLSVPYDDWDKDADPQSHHLFDRHVSSSVRLNDQDL
DILVKALNSASNPAIVLGPDVDAANANADCVMLAERLKAPVWVAPSAPRCPFPTRHPCFRGLMPAGI
AAISQLLEGHDVVLVIGAPVFRYHRYDPGQYLKPGTRLISVTCDPLEAARAPMGDAIVADIGAMASAL
ANLVEESSRQLPTAAPEPAKVDQDAGRLHPETVFDTLNDMAPENAIYLNESTSTTAQMWQRLDMR
NPGSYYFCAAGGLGFALPAAIGVQLAEPGRQVIAVIGDGSANYSISALWTAAQYNIPTIFVIMNNGTY
GLLRWSAGVLGAENVPGLDVPGIDFRALAKGYGVQALKADNLEQLKGSLQEALSAKGPVLIEVSTVSP
VK
Enzyme No 22:
(SEQ ID NO: 122)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYRQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYNCAAGGLGFALPAAIGVQLAEPERQVIAVIGDGSANYSISALWTAAQYNIPTIFVIMNNG
TYGLLRWFAGVLEAENVPGLDVPGIDFRALAKGYGVQALKADNLEQLKGSLQEALSAKGPVLIEVSTV
SPVK
Enzyme No 23:
(SEQ ID NO: 123)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYRQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYWCAAGGLGFALPAAIGVQLAEPERQVIAVIGDGSANYSISALWTAAQYNIPTIFVIMNN
GTYGALRWFAGVLEAENVPGLDVPGIDFRALAKGYGVQALKADNLEQLKGSLQEALSAKGPVLIEVST
VSPVK
Enzyme No 24:
(SEQ ID NO: 124)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYRQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYRCAAGGVGFALPAAIGVQLAEPERQVIAVIGDGSANYSISALWTAAQYNIPTIFVIMNN
GTYGALRWFAGVLEAENVPGLDVPGIDFRALAKGYGVQALKADNLEQLKGSLQEALSAKGPVLIEVST
VSPVK
Enzyme No 25:
(SEQ ID NO: 125)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYRQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYWCAAGGLGFALPAAIGVQLAEPERQVIAVIGDGSANYSISALWTAAQYNIPTIFVIMNN
GTYGNLRWFAGVLEAENVPGLDVPGIDFRALAKGYGVQALKADNLEQLKGSLQEALSAKGPVLIEVST
VSPVK
Enzyme No 26:
(SEQ ID NO: 126)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVTSSVRLNDQ
DLDILVKALNSASNPVIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYFCAAGGLGFALPAAIGVQLAEPERQVIAVIGDGSANYSISALWTAAQYNIPTIFVIMNNG
TYGTLRWFAGVLEAENVPGLDVPGIDFRALAKGYGVQALKADNLEQLKGSLQEALSAKGPVLIEVSTV
SPVK
Enzyme No 27:
(SEQ ID NO: 127)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYACAAGGLGFALPAAIGVQLAEPERQVIAVIGDGSANYSISALWTAAQYNIPTIFVIMNNG
TYGTLRWFAGVLEAENVPGLDVPGIDFRALAKGYGVQALKADNLEQLKGSLQEALSAKGPVLIEVSTV
SPVK
Enzyme No 28:
(SEQ ID NO: 128)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYLCAAGGLGFALPAAIGVQLAEPERQVIAVIGDGSANYSISALWTAAQYNIPTIFVIMNNG
TYGTLRWFAGVLEAENVPGLDVPGIDFRALAKGYGVQALKADNLEQLKGSLQEALSAKGPVLIEVSTV
SPVK
Enzyme No 29:
(SEQ ID NO: 129)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYRCAAGGVGFALPAAIGVQLAEPERQVIAVIGDGSANYSISALWTAAQYNVPTIFVIMNN
GTYGALRWFAGVLEAENVPGLDVPGIDFRALAKGYGVQALKADNLEQLKGSLQEALSAKGPVLIEVST
VSPVK
Enzyme No 30:
(SEQ ID NO: 130)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYRCAAGGVGFALPAAIGVQLAEPERQVIAVIGDGSANYSISALWTAAQYNVPTIFVIMNN
GTYGGLRWFAGVLEAENVPGLDVPGIDFRALAKGYGVQALKADNLEQLKGSLQEALSAKGPVLIEVST
VSPVK
Enzyme No 31:
(SEQ ID NO: 131)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYWCAAGGSGFALPAAIGVQLAEPERQVIAVIGDGSANYSISALWTAAQYNVPTIFVIMNN
GTYGNLRWFAGVLEAENVPGLDVPGIDFRALAKGYGVQALKADNLEQLKGSLQEALSAKGPVLIEVST
VSPVK
Enzyme No 32:
(SEQ ID NO: 132)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYRQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYWCAAGGLGFALPAAIGVQLAEPERQVIAVIGDGSANYSISALWTAAQYNIPTIFVIMNN
GTYGLLRWFAGVLEAENVPGLDVPGIDFRALAKGYGVQALKADNLEQLKGSLQEALSAKGPVLIEVST
VSPVK
Enzyme No 33:
(SEQ ID NO: 133)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYRQYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYRCAAGGLGFALPAAIGVQLAEPERQVIAVIGDGSANYSISALWTAAQYNIPTIFVIMNNG
TYGLLRWFAGVLEAENVPGLDVPGIDFRALAKGYGVQALKADNLEQLKGSLQEALSAKGPVLIEVSTV
SPVK
Enzyme No 34:
(SEQ ID NO: 134)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYWCAAGGLGFALPAAIGVQLAEPERQVIAVIGDGSANYSISALWTAAQYNIPTIFVIMNN
GTYGTLRWFAGVLEAENVPGLDVPGIDFRALAKGYGVQALKADNLEQLKGSLQEALSAKGPVLIEVST
VSPVK
Enzyme No 35:
(SEQ ID NO: 135)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPENAIYLNESTSTTAQMWQRLN
MRNPGSYYRCAAGGLGFALPAAIGVQLAEPERQVIAVIGDGSANYSISALWTAAQYNIPTIFVIMNNG
TYGTLRWFAGVLEAENVPGLDVPGIDFRALAKGYGVQALKADNLEQLKGSLQEALSAKGPVLIEVSTV
SPVK
Enzyme No 36:
(SEQ ID NO: 136)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYRCAAGGVGFALPAAIGVQLAEPERQVIAVIGDGSANYSISALWTAAQYNVPTIFVIMNN
GTYGTLRWFAGVLEAENVPGLDVPGIDFRALAKGYGVQALKADNLEQLKGSLQEALSAKGPVLIEVST
VSPVK
Enzyme No 37:
(SEQ ID NO: 137)
MASVHGTTYELLRRQGIDTVFGNPGSNELPFLKDFPEDFRYILALQEACVVGIADGYAQ
ASRKPAFINLHSAAGTGNAMGALSNAWNSHSPLIVTAGQQTRAMIGVEARLTNVDAANLPRPLVK
WSYEPASAAEVPHAMSRAIHMASMAPQGPVYLSVPYDDWDKDADPQSHHLFDRHVSSSVRLNDQ
DLDILVKALNSASNPAIVLGPDVDAANANADCVMLAERLKAPVWVAPSAPRCPFPTRHPCFRGLMP
AGIAAISQLLEGHDVVLVIGAPVFRYHRYDPGQYLKPGTRLISVTCDPLEAARAPMGDAIVADIGAMA
SALANLVEESSRQLPTAAPEPAKVDQDAGRLHPETVFDTLNDMAPEDAIYLNESTSTTAQMWQRLN
MRNPGSYYWCAAGGSGFALPAAIGVQLAEPERQVIAVIGDGSANYSISALWTAAQYNVPTIFVIMNN
GTYGTLRWFAGVLEAENVPGLDVPGIDFRALAKGYGVQALKADNLEQLKGSLQEALSAKGPVLIEVST
VSPVK

When one of the enzymes of the invention of sequence SEQ ID NO: 105 as described here-above was expressed in the Δfms1-Pyd4lk strain, this yeast was able to grow on a pantothenate free medium.

These in vivo results show that the enzymes of sequence SEQ ID NO: 105 of the invention catalyze the transformation of oxaloacetate into malonic semi-aldehyde or one of its derivatives, such as for example malonate semi-aldehyde.

B. Assays were additionally performed to analyse the kinetic properties of enzymes of the invention through the measure of their Km and Vmax, using extracts of yeasts expressing the different variants.

The kinetic assays were carried out with 100 μg of yeast extracts for 30 minutes in the presence of increasing concentrations of oxaloacetate (2.5, 5, 10, 20, 40, 80 and 120 mM), of purified YdfG (NADP-dependent 3-hydroxy acid hydrogenase—EC 1.1.1.298) from E. coli (4 μg/100 μL) and 2 mM NADPH.

The efficiency of the enzymes of the invention was assayed through the formation of 3-HP that was measured by GC/MS/MS after derivatization with BSTFA (N,O-bis(trimethyl silyl)trifluoroacetamide).

As negative control, this assay was performed on a yeast extract not comprising an enzyme according to the invention. No significant activity was detected.

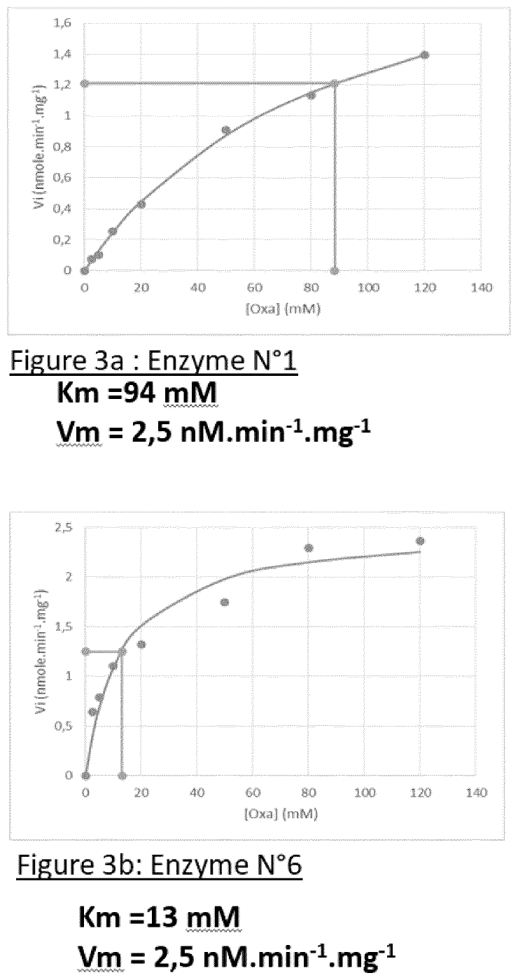

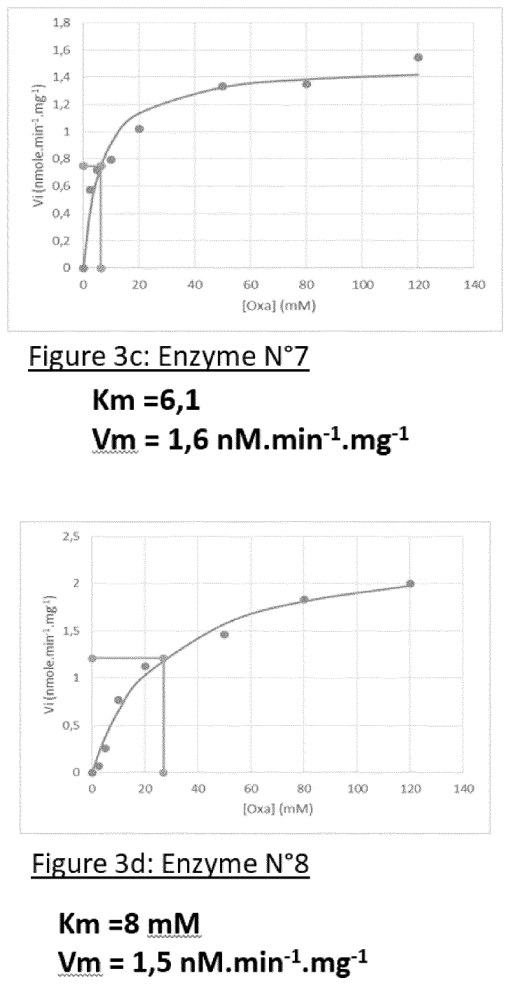

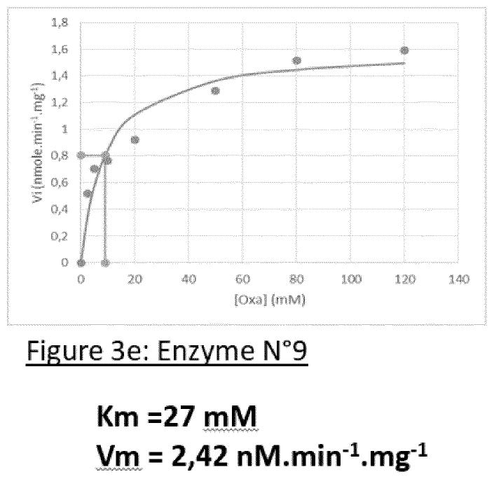

The results obtained with yeast strains comprising either enzyme No 1, enzyme No 6, enzyme No 7, enzyme No 8 or enzyme No 9 are in particular represented, respectively, in FIGS. 3a, 3b, 3c, 3d and 3e.

It can be observed that very low Km are obtained, demonstrating that the enzymes of the invention are very effective.

1. A recombinant yeast, in the genome of which at least one nucleic acid encoding an enzyme able to catalyze the decarboxylation of oxaloacetate into malonic semi-aldehyde, or one of its salts, is
(i) overexpressed and/or
(ii) under the control of a strong or inducible promoter;
said enzyme being of sequence SEQ ID NO:105:
(SEQ ID NO: 105)
MASVHGTTYELLRRQGIDX₈VFGNPGSNELPFLKDFPEDFRYILALQEACV
VGIADGYAQASRKPAFINLHSAAGTGNAMGALSNAWNSHSPLIVTAGQQTR
AMIGVEAX₁X₂TNVDAANLPRPLVKWSYEPASAAEVPHAMSRAIHMASMAP
QGPVYLSVPYDDWDKDADPQSHHLFDRHVX₉SSVRLNDQDLDILVKALNSA
SNPX₁₀IVLGPDVDAANANADCVMLAERLKAPVWVAPSAPRCPFPTRHPCF
RGLMPAGIAAISQLLEGHDVVLVIGAPVFRYX₁₁X₁₂YDPGQYLKPGTRLI
SVTCDPLEAARAPMGDAIVADIGAMASALANLVEESSRQLPTAAPEPAKVD
QDAGRLHPETVFDTLNDMAPEX₁₃AIYLNESX₃STTAQMWQRLX₁₄MRNPG
SYYX₄X₅AAGGX₆GFALPAAIGVQLAEPX₁₅RQVIAVIGDGSANYSISALW
TAAQYNX₁₆PTIFVIMNNGTYGX₇LRWX₁₇AGVLX₁₈AENVPGLDVPGIDF
RALAKGYGVQALKADNLEQLKGSLQEALSAKGPVLIEVSTVSPVK
wherein:
X₁ represents an amino acid selected from the group consisting of leucine, lysine, arginine and valine;
X₂ represents an amino acid selected from the group consisting of leucine and lysine;
X₃ represents an amino acid selected from the group consisting of threonine and serine;
X₄ represents an amino acid selected from the group consisting of phenylalanine, asparagine, alanine, isoleucine, valine, leucine, tryptophan and arginine;
X₅ represents an amino acid selected from the group consisting of cysteine and arginine;
X₆ represents an amino acid selected from the group consisting of leucine, asparagine, alanine, valine and serine;
X₇ represents an amino acid selected from the group consisting of alanine, leucine, threonine, glycine and asparagine;
X₈ represents an amino acid selected from the group consisting of threonine and isoleucine;
X₉ represents an amino acid selected from the group consisting of serine and threonine;
X₁₀ represents an amino acid selected from the group consisting of alanine and valine;
X₁₁ represents an amino acid selected from the group consisting of histidine and arginine;
X₁₂ represents an amino acid selected from the group consisting of glutamine and arginine;
X₁₃ represents an amino acid selected from the group consisting of asparagine and aspartic acid;
X₁₄ represents an amino acid selected from the group consisting of asparagine and aspartic acid;
X₁₅ represents an amino acid selected from the group consisting of glutamic acid and glycine;
X₁₆ represents an amino acid selected from the group consisting of isoleucine and valine;
X₁₇ represents an amino acid selected from the group consisting of phenylalanine and serine; and
X₁₈ represents an amino acid selected from the group consisting of glutamic acid and glycine;
with the proviso that the enzyme cannot have the sequence SEQ ID NO: 105 wherein X₁ represents leucine; X₂ represents leucine; X₃ represents threonine; X₄ represents phenylalanine; X₅ represents cysteine; X₆ represents leucine; X₇ represents alanine, X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine and X₁₈ represents glutamic acid.

2. The recombinant yeast according to claim 1, in which the said enzyme is of sequence SEQ ID NO:1
(SEQ ID NO: 1)
MASVHGTTYELLRRQGIDTVFGNPGSNELPFLKDFPEDFRYILALQEACVV
GIADGYAQASRKPAFINLHSAAGTGNAMGALSNAWNSHSPLIVTAGQQTRA
MIGVEAX₁X₂TNVDAANLPRPLVKWSYEPASAAEVPHAMSRAIHMASMAPQ
GPVYLSVPYDDWDKDADPQSHHLFDRHVSSSVRLNDQDLDILVKALNSASN
PAIVLGPDVDAANANADCVMLAERLKAPVWVAPSAPRCPFPTRHPCFRGLM
PAGIAAISQLLEGHDVVLVIGAPVFRYHQYDPGQYLKPGTRLISVTCDPLE
AARAPMGDAIVADIGAMASALANLVEESSRQLPTAAPEPAKVDQDAGRLHP
ETVFDTLNDMAPENAIYLNESX₃STTAQMWQRLNMRNPGSYYX₄X₅AAGG
X₆GFALPAAIGVQLAEPERQVIAVIGDGSANYSISALWTAAQYNIPTIFVI
MNNGTYGX₇LRWFAGVLEAENVPGLDVPGIDFRALAKGYGVQALKADNLEQ
LKGSLQEALSAKGPVLIEVSTVSPVK
wherein:
X₁ represents an amino acid selected from the group consisting of leucine, lysine, arginine and valine;
X₂ represents an amino acid selected from the group consisting of leucine and lysine;
X₃ represents an amino acid selected from the group consisting of threonine and serine;
X₄ represents an amino acid selected from the group consisting of phenylalanine, asparagine, alanine, isoleucine and valine;
X₅ represents an amino acid selected from the group consisting of cysteine and arginine;
X₆ represents an amino acid selected from the group consisting of leucine, asparagine and alanine; and
X₇ represents an amino acid selected from the group consisting of alanine and leucine, with the proviso that the enzyme cannot have the sequence SEQ ID NO: 1 wherein X₁ represents leucine; X₂ represents leucine; X₃ represents threonine; X₄ represents phenylalanine; X₅ represents cysteine; X₆ represents leucine; and X₇ represents alanine.

3. The recombinant yeast according to claim 1, in the genome of which X₁ represents an amino acid selected from the group consisting of lysine, arginine and valine, in particular from the group consisting of arginine and valine and is preferably an arginine.

4. The recombinant yeast according to claim 1, in the genome of which X₂ represents leucine.

5. The recombinant yeast according to claim 1, in the genome of which X₃ represents threonine.

6. The recombinant yeast according to claim 1, in the genome of which X₄ represents phenylalanine, asparagine or leucine, and in particular phenylalanine or leucine.

7. The recombinant yeast according to claim 1, in the genome of which X₅ represents cysteine.

8. The recombinant yeast according to claim 1, in the genome of which X₆ represents an amino acid selected from the group consisting of leucine and asparagine and is preferably leucine.

9. The recombinant yeast according to claim 1, in the genome of which X₇ represents alanine.

10. The recombinant yeast according to claim 1, in the genome of which the said enzyme is selected from the group consisting of:
(i) an enzyme of sequence SEQ ID NO: 1 wherein X₁ represents arginine; X₂ represents leucine; X₃ represents threonine; X₄ represents phenylalanine; X₅ represents cysteine; X₆ represents leucine; and X₇ represents alanine;
(ii) an enzyme of sequence SEQ ID NO: 1 wherein X₁ represents arginine; X₂ represents leucine; X₃ represents threonine; X₄ represents asparagine; X₅ represents cysteine; X₆ represents leucine; and X₇ represents alanine;
(iii) an enzyme of sequence SEQ ID NO: 1 wherein X₁ represents arginine; X₂ represents leucine; X₃ represents threonine; X₄ represents asparagine; X₅ represents cysteine; X₆ represents leucine; and X₇ represents alanine;
(iv) an enzyme of sequence SEQ ID NO: 1 wherein X₁ represents arginine; X₂ represents leucine; X₃ represents threonine; X₄ represents asparagine; X₅ represents cysteine; X₆ represents leucine; and X₇ represents leucine; and
(v) an enzyme of sequence SEQ ID NO: 1 wherein X₁ represents valine; X₂ represents lysine; X₃ represents threonine; X₄ represents asparagine; X₅ represents cysteine; X₆ represents asparagine; and X₇ represents alanine.

11. The recombinant yeast according to claim 1, wherein the strong promoter is, independently, selected from the group consisting of pTDH3, pENO2, pTEF-KI, pTEF3, pTEF1, pADH1, pGMP1, pFBA1, pPDC1, pCCW12 and pGK1.

12. The recombinant yeast according to claim 1, wherein the inducible promoter is, independently, selected from the group consisting of promoters inducible with copper, promoters inducible with methionine and promoters inducible with threonine, in particular selected from the group consisting of pSAM4, pCUP1-1, pCUP1.Cgla, pCUP1.Sba, pACUl, pACU2, pACU3p, pACU4p, pACU5, pACU6, pACU7, pACU8, pACU9, pACU10p, pACU11, pACU12, pACU13, pACU14, pACU15, pGAL/CUP1p, pCRS5, and pCHA1.

13. The recombinant yeast according to claim 1, wherein the inducible promoter is, independently, selected from the group consisting of promoters inducible due to the absence of copper, promoters inducible due to the absence of lysine and promoters inducible due to the absence of methionine, in particular selected from the group consisting of pCTR1, pCTR3, pCUR1, pCUR2, pCUR3, pCUR4, pCUR5p, pCUR6, pCUR7, pCUR8, pCUR9, pCUR10, pCUR11, pCUR12, pCUR13, pCUR14, pCUR15, pCUR16, pCUR17, pLYS1, pLYS4, pLYS9, pLYR1p, pLYR2p, pLYR3p, pLYR4p, pLYR5p, pLYR6p, pLYR7p, pLYR8, pLYR9, pLYR10, pLYR11, pMET17, pMET6, pMET14, pMET3, pSAM1, pSAM2, pMDH2, pJEN1, pICL1, pADH2 and pMLS1.

14. Method for producing malonic semi-aldehyde, one of its salts, or a malonic semi-aldehyde derivative, said method comprising the steps of:
(a) culturing a recombinant yeast as defined in claim 1 in a culture medium; and
(b) recovering the malonic semi-aldehyde, one of its salts, or a malonic semi-aldehyde derivative from said culture medium.

15. Method according to claim 14, wherein the culture medium comprises at least a carbon source, preferably a carbon source selected from the group consisting of glucose and sucrose.

16. (canceled)

17. Method according to claim 14, wherein the malonic semi-aldehyde derivative is independently selected from the group consisting of propanol, propanal, acrylic acid, acrylyl-CoA, acrylate, isopropanol, proprionate, propyonyl-CoA, 3-hydroxypropionate, 3 hydroxypropyl-CoA, 3-hydroxy-3-methyl-butyrate, phloroglucinol, flavonoids, cannabinoids, farnesyl-PP, squalene and derivatives of these pathways and is in particular selected from the group consisting of 3-hydroxypropionate, propanol, acrylic acid, acrylate, isopropanol and proprionate.

18. An enzyme characterized in that its sequence is SEQ ID NO:105:
(SEQ ID NO: 105)
MASVHGTTYELLRRQGIDX₈VFGNPGSNELPFLKDFPEDFRYILALQEACV
VGIADGYAQASRKPAFINLHSAAGTGNAMGALSNAWNSHSPLIVTAGQQTR
AMIGVEAX₁X₂TNVDAANLPRPLVKWSYEPASAAEVPHAMSRAIHMASMAP
QGPVYLSVPYDDWDKDADPQSHHLFDRHVX₉SSVRLNDQDLDILVKALNSA
SNPX₁₀IVLGPDVDAANANADCVMLAERLKAPVWVAPSAPRCPFPTRHPCF
RGLMPAGIAAISQLLEGHDVVLVIGAPVFRYX₁₁X₁₂YDPGQYLKPGTRLI
SVTCDPLEAARAPMGDAIVADIGAMASALANLVEESSRQLPTAAPEPAKVD
QDAGRLHPETVFDTLNDMAPEX₁₃AIYLNESX₃STTAQMWQRLX₁₄MRNPG
SYYX₄X₅AAGGX₆GFALPAAIGVQLAEPX₁₅RQVIAVIGDGSANYSISALW
TAAQYNX₁₆PTIFVIMNNGTYGX₇LRWX₁₇AGVLX₁₈AENVPGLDVPGIDF
RALAKGYGVQALKADNLEQLKGSLQEALSAKGPVLIEVSTVSPVK
wherein:
X₁ represents an amino acid selected from the group consisting of leucine, lysine, arginine and valine;
X₂ represents an amino acid selected from the group consisting of leucine and lysine;
X₃ represents an amino acid selected from the group consisting of threonine and serine;
X₄ represents an amino acid selected from the group consisting of phenylalanine, asparagine, alanine, isoleucine, valine, leucine, tryptophan and arginine;
X₅ represents an amino acid selected from the group consisting of cysteine and arginine;
X₆ represents an amino acid selected from the group consisting of leucine, asparagine, alanine, valine and serine;
X₇ represents an amino acid selected from the group consisting of alanine, leucine, threonine, glycine and asparagine;
X₈ represents an amino acid selected from the group consisting of threonine or isoleucine;
X₉ represents an amino acid selected from the group consisting of serine or threonine;
X₁₀ represents an amino acid selected from the group consisting of alanine or valine;
X₁₁ represents an amino acid selected from the group consisting of histidine or arginine;
X₁₂ represents an amino acid selected from the group consisting of glutamine or arginine;
X₁₃ represents an amino acid selected from the group consisting of asparagine or aspartic acid;
X₁₄ represents an amino acid selected from the group consisting of asparagine or aspartic acid;
X₁₅ represents an amino acid selected from the group consisting of glutamic acid or glycine;
X₁₆ represents an amino acid selected from the group consisting of isoleucine or valine;
X₁₇ represents an amino acid selected from the group consisting of phenylalanine or serine; and
X₁₈ represents an amino acid selected from the group consisting of glutamic acid or glycine;
with the proviso that the enzyme cannot have the sequence SEQ ID NO: 105 wherein X₁ represents leucine; X₂ represents leucine; X₃ represents threonine; X₄ represents phenylalanine; X₅ represents cysteine; X₆ represents leucine; X₇ represents alanine, X₈ represents threonine; X₉ represents serine; X₁₀ represents alanine; X₁₁ represents histidine; X₁₂ represents glutamine; X₁₃ represents asparagine; X₁₄ represents asparagine; X₁₅ represents glutamic acid; X₁₆ represents isoleucine; X₁₇ represents phenylalanine and X₁₈ represents glutamic acid.

19. The enzyme according to claim 18, having the sequence SEQ ID NO:1:
(SEQ ID NO: 1)
MASVHGTTYELLRRQGIDTVFGNPGSNELPFLKDFPEDFRYILALQEACVV
GIADGYAQASRKPAFINLHSAAGTGNAMGALSNAWNSHSPLIVTAGQQTRA
MIGVEAX₁X₂TNVDAANLPRPLVKWSYEPASAAEVPHAMSRAIHMASMAPQ
GPVYLSVPYDDWDKDADPQSHHLFDRHVSSSVRLNDQDLDILVKALNSASN
PAIVLGPDVDAANANADCVMLAERLKAPVWVAPSAPRCPFPTRHPCFRGLM
PAGIAAISQLLEGHDVVLVIGAPVFRYHQYDPGQYLKPGTRLISVTCDPLE
AARAPMGDAIVADIGAMASALANLVEESSRQLPTAAPEPAKVDQDAGRLHP
ETVFDTLNDMAPENAIYLNESX₃STTAQMWQRLNMRNPGSYYX₄X₅AAGG
X₆GFALPAAIGVQLAEPERQVIAVIGDGSANYSISALWTAAQYNIPTIFVI
MNNGTYGX₇LRWFAGVLEAENVPGLDVPGIDFRALAKGYGVQALKADNLEQ
LKGSLQEALSAKGPVLIEVSTVSPVK
wherein:
X₁ represents an amino acid selected from the group consisting of leucine, lysine, arginine and valine;
X₂ represents an amino acid selected from the group consisting of leucine and lysine;
X₃ represents an amino acid selected from the group consisting of threonine and serine;
X₄ represents an amino acid selected from the group consisting of phenylalanine, asparagine, alanine, isoleucine and valine;
X₅ represents an amino acid selected from the group consisting of cysteine and arginine;
X₆ represents an amino acid selected from the group consisting of leucine, asparagine and alanine; and
X₇ represents an amino acid selected from the group consisting of alanine and leucine,
with the proviso that the enzyme cannot have the sequence SEQ ID NO: 1 wherein X₁ represents leucine; X₂ represents leucine; X₃ represents threonine; X₄ represents phenylalanine; X₅ represents cysteine; X₆ represents leucine; and X₇ represents alanine.
